(19) United States
(12) Patent Application Publication
(10) Pub. No.: US 2024/0191300 A1
(43) Pub. Date: Jun. 13, 2024

(54) PROFILING CELL TYPES IN CIRCULATING NUCLEIC ACID LIQUID BIOPSY

(71) Applicants: THE BOARD OF TRUSTEES OF THE LELAND STANFORD JUNIOR UNIVERSITY, Stanford, CA (US); CZ BIOHUB SF, LLC, San Francisco, CA (US)

(72) Inventors: Sevahn K. Vorperian, Stanford, CA (US); Mira N. Moufarrej, Stanford, CA (US); Stephen R. Quake, Stanford, CA (US)

(21) Appl. No.: 18/286,685

(22) PCT Filed: Apr. 12, 2022
(86) PCT No.: PCT/US22/24429

§ 371 Date: Oct. 12, 2023

Related U.S. Application Data
(60) Provisional application No. 63/174,447, filed on Apr. 13, 2021.

(51) Int. Cl. C12Q 1/6883

(52) U.S. Cl. CPC C12Q 1/6883

(57) ABSTRACT

Methods and compositions for detecting specific cell types using cell-free RNA (cfRNA) are provided. In some embodiments, methods of evaluating tissue or organ function and methods of treatment are provided comprising detecting specific cell types using cfRNA.

### BACKGROUND OF THE INVENTION

Cell-free RNA (cfRNA) in blood plasma enables dynamic and longitudinal phenotypic insight into diverse physiological conditions, spanning oncology and bone marrow transplantation¹, obstetrics^{2,3}, neurodegeneration⁴, and liver disease⁵. Liquid biopsies that measure cfRNA afford broad clinical utility since cfRNA represents a mixture of transcripts that reflects the health status of multiple tissues. However, several aspects about the physiologic origins of cfRNA including the contributing cell types-of-origin remain unknown, and most current assays focus on tissue level contributions^{2,5}. Although information about tissue-of-origin can provide insight into transcriptional changes at a disease site, it would be even more powerful to incorporate knowledge from cellular pathophysiology which often forms the basis of disease⁶. This would also more closely match the resolution afforded by invasive biopsy.

### BRIEF SUMMARY OF THE INVENTION

In some embodiments, a method of evaluating the status of a cell type in a human is provided, the method comprising, providing a biological sample from the human, detecting from the biological sample the presence, absence or quantity of cell-free RNA (cfRNA) from at least 3, 4, 5, 6, 7, 8, 9, 10, 11, 12, 13, 14, 15 or more or all indicative genes, wherein the cell type and indicative genes are selected from any one of Tables 1, 2, 3, 4, or 5; and generating a score based on detection of the cfRNA from the indicative genes. In another embodiment, the method further comprises comparing the score to a control value. In another embodiment, the control value is based on a set of control subjects. In still another embodiment, the method comprises comparing the score to a prior score from an earlier-obtained biological sample from the human.

In yet another embodiment, an aforementioned method is provided further comprising detecting from the biological sample the presence, absence or quantity of cell-free RNA (cfRNA) from at least 3, 4, 5, 6, 7, 8, 9, 10, 11, 12, 13, 14, 15 or more or all indicative genes for a second cell type, wherein the indicative genes are selected from any one of Tables 1-5; generating a second score based on detection of the cfRNA from the indicative genes for the second cell type; and comparing or normalizing the score to the second score. In another embodiment, an aformentioned method is provided further comprising starting, stopping or changing a treatment of the human based on the comparing.

The present disclosure also provides, in some embodiments, a method of treating a disease or disorder in a human subject, the method comprising evaluating the status of a cell type in the human according to an aforementioned method, and administering at least one therapeutic agent or treatment to the human. As used herein, the methods of treating further optionally include methods of monitoring the progression of a disease or disorder, and optionally the method of monitoring the efficacy of a drug or treatment regimen, including, for example chemotherapy, and optionally further including stratifying a disease or disorder including, for example, determining a placement of a patient into a clinical trial.

In one embodiment, an aforementioned method is provided wherein the score is the sum of cfRNA copies detected for the indicative genes.

In some embodiments, a method is provided herein wherein the biological sample is blood, urine, cerebrospinal fluid, interstitial fluid, amniotic fluid, cord blood and/or semen. Additional biological samples include, but are not limited to, saliva, feces, and tears.

The present disclosure also provides, in one embodiment, a method of evaluating kidney function in a human, the method comprising, providing a biological sample from the human, detecting from the biological sample the presence, absence or quantity of cell-free RNA (cfRNA) from at least 3, 4, 5, 6, 7, 8, 9, 10, 11, 12, 13, 14, 15 or more or all indicative genes, wherein cell type and indicative genes are provided in Tables 1-5 and/or Table 11; generating a score based on detection of the cfRNA from the cell type and indicative genes; comparing the score to a control value or a prior score from an earlier-obtained biological sample or from a score for a different cell type from the human, thereby evaluating kidney function in the human. In another embodiment, an aforementioned method is provided wherein the providing the biological sample from the human is non-invasive.

In another embodiment, the kidney function is indicative of prognosis or diagnosis for chronic kidney disease (CKD), acute kidney injury (AKI), and/or minimal change disease. In still another embodiment, the control value is based on a set of control subjects. In yet another embodiment, an aforementioned method is provided comprising starting, stopping or changing a treatment or diagnosis, including diagnosed stage, of the human based on the comparing. In yet another embodiment, the score is the sum of cfRNA copies detected for the indicative genes. In another embodiment, an aforementioned method is provided wherein the biological sample is blood or urine. In another embodiment, an aforementioned method is provided wherein the comparing comprises comparing the score to a different cell type that is an intercalated cell, principal cell, loop of Henle cell, fibroblast, proximal tubule, podocyte, or hepatocyte. In another embodiment, an aforementioned method is provided further comprising detecting serum creatinine, urine creatinine, urine protein, cystatin C, albuminuria, and/or glomerular filtration rate (GFR) and/or estimated glomerular filtration rate (eGFR) in the human.

The present disclosure also provides, in one embodiment, a method of treating a kidney disease or disorder in a human patient, the method comprising evaluating kidney function in the human according to an aforementioned method, and administering at least one therapeutic agent or treatment to the human.

In still another embodiment, the present disclosure provides a method of evaluating brain function in a human, the method comprising, providing a biological sample from the human, detecting from the biological sample the presence, absence or quantity of cell-free RNA (cfRNA) from at least 3, 4, 5, 6, 7, 8, 9, 10, 11, 12, 13, 14, 15 or more or all indicative genes, wherein cell type and indicative genes are provided in Tables 1-5 and/or Table 6 or Table 8; generating a score based on detection of the cfRNA from the cell type and indicative genes; comparing the score to a control value or a prior score from an earlier-obtained biological sample from a score for a different cell type from the human, thereby evaluating brain function in the human.

In another embodiment, an aforementioned method is provided wherein the providing the biological sample from the human is non-invasive. In another embodiment, an aforementioned method is provided wherein the brain function is indicative of prognosis or diagnosis for Alzheimer's disease. In another embodiment, an aforementioned method is provided wherein the control value is based on a set of control subjects. In still another embodiment, an aforementioned method is provided further comprising starting, stopping or changing treatment of the human based on the comparing. In yet another embodiment, an aforementioned method is provided wherein the score is the sum of cfRNA copies detected for the indicative genes. In another embodiment, an aforementioned method is provided wherein the biological sample is blood or cerebrospinal fluid. In another embodiment, an aforementioned method is provided wherein the comparing comprises comparing the score to a different cell type that is a glial (e.g. oligodendrocyte, astrocyte, oligodendrocyte precursor cell) or neuronal cell type (e.g., inhibitory or excitatory neurons). In another embodiment, an aforementioned method is provided further comprising detecting or measuring congnition, Tau and/or amyloid beta in the human.

In another embodiment, a method of treating a brain disease or disorder in a human patient, the method comprising evaluating brain function in the human according to an aforementioned method, and administering at least one therapeutic agent or treatment to the human.

The present disclosure further provides, in one embodiment, a method of evaluating liver function in a human, the method comprising, providing a biological sample from the human, detecting from the biological sample the presence, absence or quantity of cell-free RNA (cfRNA) from at least 3, 4, 5, 6, 7, 8, 9, 10, or all indicative genes, wherein cell type and indicative genes are provided in Tables 1-5 and/or Table 12; generating a score based on detection of the cfRNA from the cell type and indicative genes; comparing the score to a control value or a prior score from an earlier-obtained biological sample from a score for a different cell type from the human, thereby evaluating liver function in the human.

In another embodiment, an aforementioned method is provided wherein the providing the biological sample from the human is non-invasive. In another embodiment, an aforementioned method is provided wherein the liver function is indicative of prognosis or diagnosis for non-alcoholic fatty liver disease, non-alcoholic steatohepatitis, and/or liver cancer. In another embodiment, an aforementioned method is provided wherein the control value is based on a set of control subjects. In another embodiment, an aforementioned method is provided further comprising starting, stopping or changing a treatment of the human based on the comparing. In another embodiment, an aforementioned method is provided wherein the score is the sum of cfRNA copies detected for the indicative genes. In another embodiment, an aforementioned method is provided wherein the biological sample is blood or urine. In another embodiment, an aforementioned method is provided wherein the comparing comprises comparing the score to a different cell type that is a liver sinusoidal endothelial cell, a kidney cell, a neutrophil, an cosinophil, or a basophil. In another embodiment, an aforementioned method is provided further comprising detecting Alanine transaminase (ALT), Aspartate transaminase (AST), Alkaline phosphatase (ALP), Albumin, total protein, Bilirubin, Gamma-glutamyltransferase (GGT), L-lactate dehydrogenase (LD), and/or Prothrombin time in the human.

In another embodiment, the present disclosure provides a method of treating a liver disease or disorder in a human patient, the method comprising evaluating liver function in the human according to an aforementioned method, and administering at least one therapeutic agent or treatment to the human.

In still another embodiment, the present disclosure provides a non-transitory computer-readable storage device storing computer-executable instructions that, in response to execution, cause a processor to perform operations, the operations comprising: receiving data indicating presence, absence or quantity of cell-free RNA (cfRNA) from at least 3, 4, 5, 6, 7, 8, 9, 10, 11, 12, 13, 14, 15, or more or all indicative genes for a cell type, wherein indicative genes are selected from any one of Table 1, 2, 3, 4, or 5; generating a score based on detection of the cfRNA from the indicative genes; comparing the score to a control value or a prior score from an earlier-obtained biological sample from the human, upon determining that the score is above or below the control value or prior score, generating a classification of disease or prognosis of the human related to the cell type; and displaying the classification.

### BRIEF DESCRIPTION OF THE DRAWINGS

FIGS. 1A-1E: Cell type decomposition of the plasma cell free transcriptome using Tabula Sapiens. FIG. 1a: Integration of tissue-of-origin and single cell transcriptomics to identify cell types-of-origin in cfRNA. FIG. 1b: Cell type specific markers defined in context of the human body identified in plasma cfRNA. Error bars denote the s.d. of number of cell type specific markers (n=75 patients); the measure of center is the mean. CPM-TMM counts for a given gene across technical replicates were averaged prior to intersection. FIG. 1c: Cluster heatmap of Spearman correlations of cell type basis matrix column space derived from Tabula Sapiens. Color bar denotes correlation value. FIG. 1d: Mean fractional contributions of cell type specific RNA in plasma cf-transcriptome (n=18 patients). FIG. 1e: Top tissues in cfRNA not captured by basis matrix (e.g. the set difference of all genes detected in a given cfRNA sample and the row space of the basis matrix intersection with HPA tissue specific genes). Error bars denote the s.d. of number of HPA tissue specific genes with NX counts>10 and cell free CPM expression≥ 1 (n=18 patients); the measure of center is the mean.

FIGS. 2A-2D: Cellular pathophysiology is noninvasively resolvable in cfRNA. For a given boxplot, any cell type signature score is the sum of log-transformed CPM-TMM normalized counts. The horizontal line denotes the median; lower hinge, 25^{th} percentile; upper hinge, 75^{th} percentile; whiskers, 1.5 interquartile range; points outside whiskers indicate outliers. All P values were determined by a Mann Whitney U test; sidedness specified in subplot caption. *P<0.05. ** P<10⁻². *** P<10⁻⁴. **** P<10⁻⁵. FIG. 2a: Neuronal and glial cell type signature scores in healthy cfRNA plasma (n=18) on a logarithmic scale. Excitatory Neuron, Ex; Oligodendrocyte, Oli; Astrocyte, Ast; Oligodendrocyte Precursor Cell, Opc; Inhibitory Neuron, In. FIG. 2b: Comparison of the proximal tubule signature score in CKD stages 3+(n=51 samples; 9 patients) and healthy controls (n=9 samples; 3 patients) (P=9.66*10⁻³, U=116, one-sided). Dot color denotes each patient. FIG. 2c: Hepatocyte signature score between healthy (n=16) and both NAFLD (n=46) (P=3.15×10⁻⁴, U=155, one-sided) and NASH (n=163) (P=4.68×10⁻⁶, U=427, one-sided); NASH vs. NAFLD (P=0.464, U=3483, two-sided). Color reflects sample collection center. FIG. 2d: Neuronal and glial signature scores in AD (n=40) and NCI (n=18) cohorts. Excitatory neuron (P=4.94×10⁻³, U=206, one-sided), oligodendrocyte (P=2.28×10⁻³, U=178, two-sided), oligodendrocyte progenitor (P=2.27×10⁻², U=224, two-sided), and astrocyte (P=6.11×10⁻⁵, U=121, two-sided). Acronyms corresponds to those in (a).

FIGS. 3A-3C: Cell-free RNA Sample Quality Control. Quality control metrics (3′ bias fraction, ribosomal fraction, and DNA contamination) were determined for each cfRNA sample downloaded from a given SRA accession number. Samples with outlier values are highlighted in red and were not considered in subsequent analyses (see Methods section ‘Sample quality filtering’). (FIG. 3a) Ibarra et al (n=285) (FIG. 3b) Toden et al (n=339) (FIG. 3c) Chalasani et al (n=500). Box plot: horizonal line, median; lower hinge, 25^{th} percentile; upper hinge, 75^{th} percentile; whiskers span the 1.5 interquartile range; points outside the whiskers indicate outliers. Each point corresponds to a downloaded cfRNA sample from the corresponding SRA accession number.

FIGS. 4A-4C: Hierarchical clustering on non-immune Tabula Sapiens organ compartments. Dashed line indicates the height at which tree was cut. Dendrograms correspond with the cell type annotations belonging to (FIG. 4a) the epithelial compartment, (FIG. 4b) the endothelial compartment (FIG. 4c) the stromal compartment.

FIGS. 5A-5B: Tabula Sapiens basis matrix performance on GTEx bulk RNA samples using nu-SVR. GTEx tissue samples possessing cell types wholly present and absent from the basis matrix column space were selected. For box plots: horizonal line, median; lower hinge, 25th percentile; upper hinge, 75th percentile; whiskers, 1.5 interquartile range; points outside the whiskers indicate outliers. There are 30 bulk RNA seq samples for a given tissue except for the Bladder (n=21), Kidney—Medulla (n=4), and Whole Blood (n=19). (FIG. 5a) Root mean square error between predicted expression and measured expression in a given GTEx tissue. Units are zero-mean unit variance scaled CPM counts. Tissues present in TSP have reduced RMSE compared to those that are absent (Kidney—Medulla and Brain). Tissues with high cellular heterogeneity (for example Lung, Bladder, Small Intestine, Kidney) exhibit reduced deconvolution performance compared to less heterogeneous tissues (for example Whole Blood, Spleen, Liver). (FIG. 5b) Pearson correlation between predicted expression and measured expression in a given GTEx tissue.

FIGS. 6A-6C: Deconvolution of healthy plasma samples from Toden et al using Tabula Sapiens. Pie charts denote mean fractional cell type specific RNA contributions for (FIG. 6a) University of Indiana (n=17), (FIG. 6b) University of Kentucky (n=18), (FIG. 6c) Washington University in St. Louis (n=22).

FIGS. 7A-7D: nuSVR decomposition of the plasma cell free transcriptome with Tabula Sapiens. For boxplots, horizonal line, median; lower hinge, 25th percentile; upper hinge, 75th percentile; whiskers span the 1.5 interquartile range; points outside the whiskers indicate outliers. Each point corresponds to a patient in a given cohort; University of Indiana (n=17), University of Kentucky (n=18), Washington University in St. Louis (n=22), and BioIVT (n=18). For heatmaps or clustermaps, the scale bar denotes the pearson correlation value. (FIG. 7a) Complete linkage clustermap of pairwise pearson correlation of deconvolved cell type fractions between patients from a given center; row color denotes a given center (n=75 patients). (FIG. 7b) Heatmap of pairwise pearson correlation of the mean cell type coefficients per center. (FIG. 7c) Deconvolution RMSE between predicted vs. measured expression for all biological replicates across all centers. (FIG. 7d) Deconvolution pearson correlation between predicted vs. measured expression for all biological replicates across all centers.

FIGS. 8A-8D: Establishing gene profile cell type specificity in context of the whole body using single cell and bulk RNA-seq data. (FIG. 8a) Cell type signature scoring procedure; please see the ‘Signature Scoring’ in the Methods for the full derivation procedure of a given cell type gene profile. (FIG. 8b) Single cell heatmaps for gene cell type profiles within the corresponding tissue cell atlas, demonstrating that a cell type specific profile is unique to a given cell type across those within a given tissue. Columns denote marker genes for a given cell type; rows indicate individual cells. The color bar scale corresponds to log-transformed counts-per-ten thousand. (FIG. 8c) Gini coefficient density plot for genes in cell type profiles derived from brain and liver single cell atlases using HPA NX counts. The area under the curve for a given cell type sums to one. (FIG. 8d) Log fold change in bulk RNA-seq data of a given cell type profile, demonstrating that the predominant expression of the cell type signature in its native tissue is highest relative to other non-native tissues. Values are the log-fold change of the signature score of a given cell type profile in the native tissue (indicated by the y-axis) to the mean expression in the remaining non-native tissues. Box plot: horizontal line, median; lower hinge, 25th percentile; upper hinge, 75th percentile; whiskers span the 1.5 interquartile range; points outside the whiskers indicate outliers (n=2462 GTEx brain samples for box plot on left; n=226 GTEx liver samples, right).

FIG. 9. Distribution of Gini coefficient and Tau for all genes denoted by HPA as specific to the brain, liver, placenta, and kidney.

FIGS. 10A-10G: Comprehensive placental and renal cell type gene profile specificity at single cell and whole body resolution. For box plots in f, g: horizontal line, median; lower hinge, 25th percentile; upper hinge, 75th percentile; whiskers span the 1.5 interquartile range; points outside whiskers indicate outliers. (FIG. 10a) Violin plot of derived syncytiotrophoblast and extravillous trophoblast gene profiles from Vento-Tormo et al. (FIG. 10b) Violin plot of derived syncytiotrophoblast and extravillous trophoblast gene profiles from Suryawanshi et al. (FIG. 10c) Violin plot of derived proximal tubule gene profile (FIG. 10d) Gini coefficient distribution for placental trophoblast cell types in (FIG. 10a) and (FIG. 10b) (FIG. 10e) Gini coefficient distribution for renal cell type in (FIG. 10c) (FIG. 10f) Distribution of placental trophoblast signature scores across all GTEx tissues. Note: given that the placenta is not in GTEx, the box plots correspond to the distribution of signature scores across non-placental tissues (sum of log-transformed counts-per-ten thousand) (n=17382 non-placenta GTEx samples) (FIG. 10g) Log-fold change of renal cell type signature score in GTEx Kidney Cortex/Medulla samples relative to the mean non-kidney signature score, demonstrating that the predominant expression of the cell type signature in its native tissue is highest relative to other non-native tissues. Values are the log ratio of the signature score in the kidney to the mean signature score in the remaining non-kidney GTEx tissue samples (n=89 GTEx renal cortex or medulla samples).

FIGS. 11A-11D: Expression distribution of Tsang et al trophoblast gene profiles in placenta scRNA atlases and in preeclampsia cfRNA. Derived trophoblast signature scores in the (FIG. 11a) iPEC dataset (mothers with no complications, n=73 patients; mothers with preeclampsia, n=40 patients) and (FIG. 11b) PEARL-PEC (n=12 patients for each early/late-onset PE cohorts and gestationally-age matched healthy controls) datasets from Munchel et al. Box plot: horizontal line, median; lower hinge, 25th percentile; upper hinge, 75th percentile; whiskers span the 1.5 interquartile range; points outside the whiskers indicate outliers. Stacked violin plot of the genes comprising the extravillous trophoblast and syncytiotrophoblast gene profiles from Tsang et al. intersecting with the measured genes in (FIG. 11c) Suryawanshi et al and (FIG. 11d) Vento-Tormo et al, reflecting the expression distribution across all observed placental cell types.

FIGS. 12A-12F: Assessment of cell type gene profile discriminatory power during signature scoring. (FIG. 12a) Density of p-values over 10,000 trial permutation test to assess p-value calibration for a given signature score. In all cases, the distribution is uniform, as expected under the null. (FIG. 12b) Density of U values over 10,000 trial permutation test; red line indicates the U value corresponding to the experimental comparison reported in FIG. 2. (FIG. 12c) Donut plot reflecting the number of genes in the hepatocyte cell type gene profile that intersect with the reported NAFLD DEG in Chalasani et al. (FIG. 12d) Density plot reflecting the Gini coefficient distribution corresponding to DEG in NAFLD that are liver or hepatocyte specific. The Gini coefficient is computed using the mean expression per liver cell type in Aizarani et al (Methods). Area under each curve sums to one. (FIG. 12e) Donut plots reflecting the number of genes in brain cell type gene profiles that intersect with the reported AD DEG in Toden et al. (FIG. 12f) Density plot reflecting the Gini coefficient distribution corresponding to DEG in AD that are brain or brain cell type specific. The Gini coefficient is computed using the mean expression per brain cell type in the ‘Normal’ samples of Mathys et al (Methods). Area under each curve sums to one.

FIGS. 13A-13J: Deconvolved fractions of cell type specific RNA from various GTEx tissues using nu-SVR and the Tabula Sapiens basis matrix. Top 20 largest fractional contributions of cell type specific RNA for a given tissue. The two tissues whose cell types were absent from the basis matrix column space were Kidney—Medulla and Brain. Kidney medulla samples reported to be contaminated with cortex are reflected by deconvolved kidney epithelia fractions. The brain, which is absent from the TSP v1.0, yields majority fractions of schwann cell-specific RNA, a peripheral nervous cell type. Majority cell types for a given tissue, such as lung pneumonocytes and immune cells in the lung or kidney epithelia for the kidney cortex underscore the ability for the basis matrix to capture representative fractions of cell type specific RNA and reflect underlying cell heterogeneity in bulk RNA-seq data. (FIG. 13a) Bladder (FIG. 13b) Brain (FIG. 13c) Colon—Transverse (FIG. 13d) Kidney—Cortex (FIG. 13e) Kidney—Medulla (FIG. 13f) Liver (FIG. 13g) Lung (FIG. 13h) Small Intestine—Terminal Ileum (FIG. 13i) Spleen (FIG. 13j) Whole Blood.

FIG. 14: Signature scores of cell types from the liver, heart, normal brain, lung, bladder, pancreas, testis, intestine, prostate, and kidney in plasma cell-free RNA (n=75 healthy patient samples) on a logarithmic scale. The signature score is the sum of log-transformed CPM-TMM normalized counts. The horizontal line denotes the median; the lower hinge indicates the 25th percentile; the upper hinge indicates the 75th percentile; whiskers indicate the 1.5 interquartile range; and points outside the whiskers indicate outliers

### DEFINITIONS

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “an agent” includes reference to one or more agents known to those skilled in the art, and so forth.

The term “cell-free RNA sample” or “cfRNA sample” refers to a nucleic acid sample comprising extracellular RNA, which nucleic acid sample is obtained from any cell-free biological fluid, for example, whole blood processed to remove cells, urine, saliva, or amniotic fluid. In some embodiments, cfRNA for analysis is obtained from whole blood processed to remove cells, e.g., a plasma or serum sample. As used herein, the terms “cell-free RNA” or “cfRNA” refer to RNA recoverable from the non-cellular fraction of a bodily fluid, such as blood (including, for example, whole blood, plasma, and/or serum), and includes fragments of full-length RNA transcripts.

The “status” of the cell type can indicate the relative health of the particular cell type or tissue or organ in the human (e.g., human subjects of all ages and fetuses). Depending on the cell type, an increase or decrease in the number of a cell type can indicate an improvement or reduction in health, and can be used, for example, to identify individuals for treatment.

As used herein, the term “function,” for example as it relates to organ function (kidney function, liver function, brain function, etc.) or the status of a cell type within an organ or tissue, refers in some embodiments to the health or condition of the organ or tissue. As will be appreciated by those of skill in the art, the methods provided herein enable the assessment of organ health or organ disease state (e.g., an indication of a functional organ or an indication of a non-functional or dysfunctional organ). The methods disclosed herein further allow the diagnosis and/or prognosis of a particular disease or disorder, as well as the ability to monitor disease progression and/or the response of a patient to certain therapeutic agents and regimens. For example, in some embodiments, certain cell types are implicated in some diseases and measuring these cell types or their differences lead to disease diagnosis.

The terms “determining,” “assessing,” “assaying,” “measuring” and “detecting” as used herein are used interchangeably and refer to quantitative determinations.

The term “amount” or “level” refers to the quantity of copies of an RNA transcript being assayed, including fragments of full-length transcripts that can be unambiguously identified as fragments of the transcript being assayed. Such quantity may be expressed as the total quantity of the RNA, in relative terms, e.g., compared to the level present in a control cfRNA sample, or as a concentration e.g., copy number per milliliter of biofluid, of the RNA in the sample.

As used herein, the term “expression level” of a gene as described herein refers to the level of expression of an RNA transcript of the gene.

The term “nucleic acid” or “polynucleotide” as used herein refers to a deoxyribonucleotide or ribonucleotide in either single- or double-stranded form. In the context of primers or probes, the term encompasses nucleic acids containing known analogues of natural nucleotides which have similar or improved binding properties, for the purposes desired, as the reference nucleic acid; and nucleic-acid-like structures with synthetic backbones.

The term “treatment,” “treat,” or “treating” typically refers to a clinical intervention, which can include one or multiple interventions over a period of time, to ameliorate at least one symptom of a disease or otherwise slow progression. This includes alleviation of symptoms, diminishment of any direct or indirect pathological consequences of a disease, amelioration of the disease, and improved prognosis. It is understood that treatment can include but does not necessarily refer to prevention of the disease. The present disclosure also provides methods for stratifying disease on the basis of cell type and using such information as a clinical biomarker, including, for example, using such biomarkers for enrollment into a drug clinical trial.

### DETAILED DESCRIPTION OF THE INVENTION

The present disclosure, in various embodiments, provides compositions and methods to detect the status of specific cell types (including, for example, the relative levels of cell types in disease compared to a healthy control sample) in a subject such as a human via detection of cfRNA from a biological sample from the human. By detecting the status of specific cell types, one can measure a disease state, function, and/or reaction or response to a drug or treatment and optionally can for example begin, end, or change a treatment or drug dosage for the human.

Contrary to other reports and methods, the present disclosure ensured that while defining a cell type gene profile a given gene is cell type specific in context of the whole body. This is because cell-free nucleic acids are derived from biofluids that interface with multiple organs (i.e. blood, entire body; urine, urinary tract). Therefore, in order to identify or associate the cell type of origins for a gene measured in cfRNA, its endogenous expression must be readily measurable in a given single cell atlas and its expression must be unique to that given cell type in context of the entire body.

Unlike prior work deriving cell type gene profiles for signature scoring in blood or plasma (US20180372726; Tsang, J. C. H. et al., Proc Natl Acad Sci USA 114, E7786-E7795 (2017); Vong, J. S. L. et al., Clinical Chemistry, 67(11):1492-1502; and Pique-Regi, R. et al., Genetics and Genomics, eLife 2019; 8:e52004 DOI: 10.7554/eLife.52004), the present disclosure considers gene expression across the whole body during the derivation of a cell type gene profile.

As described herein, by considering gene expression throughout the human body using bulk tissue data spanning over 50 tissues⁵⁰ in defining a cell type gene profile, a given cell type gene profile is not only specific to a given cell type in a given single cell atlas but also to its corresponding native tissue/organ system in context of the whole body. Cell type functions are reflected by various transcriptional programs, which can be shared between different cell types (Breschi, A., et al., Genome Research, 30:1047-159 (202); Quake, S.R., The Tabula Sapiens Consortium, bioRxiv, 2021, doi: https://doi.org/10.1101/2021.07.19.452956; and Schaum, N., et al., Nature, 562(7727): 367-372 (2018)). To this end, a specialized cell type in a given tissue may have parallel functions by other cell types in other tissues throughout the human body and this must be accounted for in the derivation of a cell type gene profile for noninvasive signature scoring in cfRNA.

The aberrant expression of asserted trophoblast genes by Tsang et al in other placental cell types underscores the importance of considering both high endogenous expression in the cell type of interest as well as gene expression throughout the body when deriving a gene profile for signature scoring in cell-free RNA, and such a methodology has not been described to date (US20180372726; Tsang, J. C. H. et al.; Vong et al.; and Romero et al.).

### Genes Indicating Cell Types

The following provides information regarding cell types and sets of genes whose cfRNAs can be used to specifically detect the status of the listed cell type. One need not use the full listing of indicative genes to detect a specific cell type, however, the strength of the signal and thus ability to optimally associate the expression from the genes to a specific cell type will benefit from an increasing number of genes as listed herein. In some embodiments, at least 3, 4, 5, 6, 7, 8, 9, 10, 11, 12, 13, 14, 15, or more or all of the genes (e.g., via cfRNA detection) are detected. The gene names in the tables and as used herein are from Ensembl version: 92.38 (Human Protein Atlas version 19). Reference to the gene is intended to include the sequence indicated as well as any human allelic variant or splice variants that are encoded by the gene.

Table 1 includes a full list of genes indicative of cell types as listed therein. As noted above, subsets of these genes can be detected to detect the indicated cell types. Tables 2-5 list a subset of cell types with a subset of indicative genes that can be used to detect the indicated cell types, albeit with an increased Gini coefficient as indicated in the Table title. The genes in Table 16 were determined by Gini coefficient greater than or equal to 0.6 as well as differentially expressed for the respective cell type in two independent placental single cell datasets (Vento-Tormo, et al., Nature volume 563, pages 347-353(2018); and Suryawanshi et al., Science Advances 31 Oct. 2018: Vol. 4, no. 10). The genes in Tables 6-18 were determined by Gini coefficient greater than or equal to 0.6.

TABLE 1
Cell types and indicative genes

| | Gene list |
|---|---|
| Early primary | ENSG00000177324 (BEND2), ENSG00000187268 (FAM9C), ENSG00000189401 |
| Spermatocyte | (OTUD6A), ENSG00000164256 (PRDM9), ENSG00000182459 (TEX19) |
| Elongated | ENSG00000183559 (C10orf120), ENSG00000173728 (C1orf100), |
| Spermatid | ENSG00000125975 (C20orf173), ENSG00000175820 (CCDC168), |
| | ENSG00000178395 (CCDC185), ENSG00000125815 (CST8), ENSG00000214866 |
| | (DCDC2C), ENSG00000172404 (DNAJB7), ENSG00000170613 (FAM71B), |
| | ENSG00000125245 (GPR18), ENSG00000214686 (IQCF6), ENSG00000205858 |
| | (LRRC72), ENSG00000165076 (PRSS37), ENSG00000258223 (PRSS58), |
| | ENSG00000181240 (SLC25A41), ENSG00000153060 (TEKT5), |
| | ENSG00000196900 (TEX43), ENSG00000124251 (TP53TG5), ENSG00000155890 |
| | (TRIM42), ENSG00000120440 (TTLL2), ENSG00000168454 (TXNDC2), |
| | ENSG00000162843 (WDR64) |
| Late primary | ENSG00000180071 (ANKRD18A), ENSG00000148513 (ANKRD30A), |
| Spermatocyte | ENSG00000169679 (BUB1), ENSG00000180116 (C12orf40), ENSG00000156509 |
| | (FBXO43), ENSG00000171989 (LDHAL6B) |
| Round Spermatid | ENSG00000164398 (ACSL6), ENSG00000134249 (ADAM30), ENSG00000120051 |
| | (CFAP58), ENSG00000212710 (CTAGE1), ENSG00000164334 (FAM170A), |
| | ENSG00000179796 (LRRC3B), ENSG00000184507 (NUTM1), ENSG00000078795 |
| | (PKD2L2) |
| spermatogonial | ENSG00000262874 (C19orf84), ENSG00000163530 (DPPA2), ENSG00000105255 |
| stem cell | (FSD1), ENSG00000187867 (PALM3), ENSG00000171794 (UTF1) |
| Sertoli Cell | ENSG00000101448 (EPPIN), ENSG00000147378 (FATE1), ENSG00000243955 |
| | (GSTA1), ENSG00000123999 (INHA), ENSG00000171864 (PRND), |
| | ENSG00000204065 (TCEAL5), ENSG00000204071 (TCEAL6) |
| Intercalated | ENSG00000147614 (ATP6V0D2), ENSG00000151418 (ATP6V1G3), |
| cell | ENSG00000109684 (CLNK), ENSG00000173253 (DMRT2), ENSG00000168269 |
| | (FOXI1), ENSG00000188175 (HEPACAM2), ENSG00000113073 (SLC4A9), |
| | ENSG00000035720 (STAP1), ENSG00000143001 (TMEM61) |
| Podocyte | ENSG00000138792 (ENPEP), ENSG00000113578 (FGF1), ENSG00000196549 |
| | (MME), ENSG00000116218 (NPHS2), ENSG00000151490 (PTPRO) |
| Principal cell | ENSG00000150201 (FXYD4), ENSG00000160951 (PTGER1), ENSG00000110693 |
| | (SOX6) |
| Proximal tubule | ENSG00000153086 (ACMSD), ENSG00000183747 (ACSM2A), |
| | ENSG00000066813 (ACSM2B), ENSG00000132744 (ACY3), ENSG00000116771 |
| | (AGMAT), ENSG00000113492 (AGXT2), ENSG00000136872 (ALDOB), |
| | ENSG00000166825 (ANPEP), ENSG00000204653 (ASPDH), ENSG00000129151 |
| | (BBOX1), ENSG00000145692 (BHMT), ENSG00000164237 (CMBL), |
| | ENSG00000205279 (CTXN3), ENSG00000107611 (CUBN), ENSG00000132437 |
| | (DDC), ENSG00000015413 (DPEP1), ENSG00000147647 (DPYS), |
| | ENSG00000162391 (FAM151A), ENSG00000010932 (FMO1), ENSG00000171766 |
| | (GATM), ENSG00000149124 (GLYAT), ENSG00000211445 (GPX3), |
| | ENSG00000243955 (GSTA1), ENSG00000244067 (GSTA2), ENSG00000116882 |
| | (HAO2), ENSG00000138030 (KHK), ENSG00000081479 (LRP2), |
| | ENSG00000100253 (MIOX), ENSG00000125144 (MT1G), ENSG00000205358 |
| | (MT1H), ENSG00000144035 (NAT8), ENSG00000086991 (NOX4), |
| | ENSG00000174827 (PDZK1), ENSG00000250799 (PRODH2), ENSG00000135069 |
| | (PSAT1), ENSG00000139194 (RBP5), ENSG00000178828 (RNF186), |
| | ENSG00000081800 (SLC13A1), ENSG00000158296 (SLC13A3), |
| | ENSG00000165449 (SLC16A9), ENSG00000124564 (SLC17A3), |
| | ENSG00000197901 (SLC22A6), ENSG00000149452 (SLC22A8), |
| | ENSG00000131183 (SLC34A1), ENSG00000148942 (SLC5A12), |
| | ENSG00000137251 (TINAG), ENSG00000171234 (UGT2B7) |
| Thick ascending limb | ENSG00000113946 (CLDN16), ENSG00000130829 (DUSP9), ENSG00000179399 |
| of Loop of Henle cell | (GPC5), ENSG00000169344 (UMOD) |
| astrocyte in alzheimers | ENSG00000100427 (MLC1), ENSG00000103740 (ACSBG1), ENSG00000111783 |
| brain | (RFX4), ENSG00000129244 (ATP1B2), ENSG00000131095 (GFAP), |
| | ENSG00000139155 (SLCO1C1), ENSG00000141469 (SLC14A1), |
| | ENSG00000146005 (PSD2), ENSG00000147509 (RGS20), ENSG00000148482 |
| | (SLC39A12), ENSG00000156076 (WIF1), ENSG00000161509 (GRIN2C), |
| | ENSG00000163285 (GABRG1), ENSG00000164089 (ETNPPL), |
| | ENSG00000165478 (HEPACAM), ENSG00000168309 (FAM107A), |
| | ENSG00000171885 (AQP4), ENSG00000179399 (GPC5), ENSG00000179796 |
| | (LRRC3B), ENSG00000182902 (SLC25A18), ENSG00000188039 (NWD1) |
| excitatory neuron in | ENSG00000011347 (SYT7), ENSG00000059915 (PSD), ENSG00000063180 |
| alzheimers brain | (CA11), ENSG00000066248 (NGEF), ENSG00000070808 (CAMK2A), |
| | ENSG00000074211 (PPP2R2C), ENSG00000102468 (HTR2A), ENSG00000103316 |
| | (CRYM), ENSG00000104722 (NEFM), ENSG00000104888 (SLC17A7), |
| | ENSG00000106089 (STX1A), ENSG00000107295 (SH3GL2), ENSG00000108309 |
| | (RUNDC3A), ENSG00000110427 (KIAA1549L), ENSG00000115423 (DNAH6), |
| | ENSG00000117152 (RGS4), ENSG00000118160 (SLC8A2), ENSG00000118733 |
| | (OLFM3), ENSG00000119042 (SATB2), ENSG00000119125 (GDA), |
| | ENSG00000121905 (HPCA), ENSG00000123119 (NECAB1), ENSG00000127585 |
| | (FBXL16), ENSG00000130558 (OLFM1), ENSG00000132872 (SYT4), |
| | ENSG00000134343 (ANO3), ENSG00000135426 (TESPA1), ENSG00000140015 |
| | (KCNH5), ENSG00000141668 (CBLN2), ENSG00000145335 (SNCA), |
| | ENSG00000149654 (CDH22), ENSG00000149970 (CNKSR2), ENSG00000150394 |
| | (CDH8), ENSG00000152822 (GRM1), ENSG00000154146 (NRGN), |
| | ENSG00000156564 (LRFN2), ENSG00000157782 (CABP1), ENSG00000158258 |
| | (CLSTN2), ENSG00000164061 (BSN), ENSG00000164076 (CAMKV), |
| | ENSG00000165023 (DIRAS2), ENSG00000166257 (SCN3B), ENSG00000168490 |
| | (PHYHIP), ENSG00000168830 (HTR1E), ENSG00000171246 (NPTX1), |
| | ENSG00000171509 (RXFP1), ENSG00000171532 (NEUROD2), |
| | ENSG00000171617 (ENC1), ENSG00000171798 (KNDC1), ENSG00000172020 |
| | (GAP43), ENSG00000174145 (NWD2), ENSG00000175874 (CREG2), |
| | ENSG00000176749 (CDK5R1), ENSG00000180354 (MTURN), ENSG00000182674 |
| | (KCNB2), ENSG00000184613 (NELL2), ENSG00000184672 (RALYL), |
| | ENSG00000185518 (SV2B), ENSG00000187122 (SLIT1), ENSG00000196353 |
| | (CPNE4) |
| inhibitory neuron in | ENSG00000004848 (ARX), ENSG00000127152 (BCL11B), ENSG00000136750 |
| alzheimers brain | (GAD2), ENSG00000151812 (SLC35F4), ENSG00000198785 (GRIN3A) |
| oligodendrocyte in | ENSG00000011426 (ANLN), ENSG00000054690 (PLEKHH1), ENSG00000084453 |
| alzheimers brain | (SLCO1A2), ENSG00000086205 (FOLH1), ENSG00000099194 (SCD), |
| | ENSG00000105695 (MAG), ENSG00000108381 (ASPA), ENSG00000117266 |
| | (CDK18), ENSG00000123560 (PLP1), ENSG00000124920 (MYRF), |
| | ENSG00000136541 (ERMN), ENSG00000140479 (PCSK6), ENSG00000147488 |
| | (ST18), ENSG00000150656 (CNDP1), ENSG00000158865 (SLC5A11), |
| | ENSG00000164124 (TMEM144), ENSG00000168314 (MOBP), ENSG00000169247 |
| | (SH3TC2), ENSG00000170775 (GPR37), ENSG00000172508 (CARNS1), |
| | ENSG00000184144 (CNTN2), ENSG00000197430 (OPALIN), ENSG00000197971 |
| | (MBP), ENSG00000204655 (MOG) |
| oligodendrocyte | ENSG00000072182 (ASIC4), ENSG00000075461 (CACNG4), ENSG00000089250 |
| progenitor cell in | (NOS1), ENSG00000101198 (NKAIN4), ENSG00000101203 (COL20A1), |
| alzheimers brain | ENSG00000114646 (CSPG5), ENSG00000118322 (ATP10B), ENSG00000132692 |
| | (BCAN), ENSG00000139352 (ASCL1), ENSG00000144230 (GPR17), |
| | ENSG00000148123 (PLPPR1), ENSG00000150361 (KLHL1), ENSG00000157890 |
| | (MEGF11), ENSG00000169181 (GSG1L), ENSG00000169302 (STK32A), |
| | ENSG00000184221 (OLIG1), ENSG00000187398 (LUZP2), ENSG00000187416 |
| | (LHFPL3), ENSG00000196132 (MYT1), ENSG00000196338 (NLGN3), |
| | ENSG00000198732 (SMOC1), ENSG00000203805 (PLPP4), ENSG00000205927 |
| | (OLIG2) |
| Hepatocytes | ENSG00000121410 (A1BG), ENSG00000183044 (ABAT), ENSG00000183747 |
| | (ACSM2A), ENSG00000183549 (ACSM5), ENSG00000187758 (ADH1A), |
| | ENSG00000196616 (ADH1B), ENSG00000198099 (ADH4), ENSG00000172955 |
| | (ADH6), ENSG00000079557 (AFM), ENSG00000172482 (AGXT), |
| | ENSG00000145192 (AHSG), ENSG00000198610 (AKR1C4), ENSG00000122787 |
| | (AKR1D1), ENSG00000144908 (ALDH1L1), ENSG00000118514 (ALDH8A1), |
| | ENSG00000136872 (ALDOB), ENSG00000214274 (ANG), ENSG00000132855 |
| | (ANGPTL3), ENSG00000138356 (AOX1), ENSG00000118137 (APOA1), |
| | ENSG00000110243 (APOA5), ENSG00000130208 (APOC1), ENSG00000110245 |
| | (APOC3), ENSG00000267467 (APOC4), ENSG00000224916 (APOC4-APOC2), |
| | ENSG00000130203 (APOE), ENSG00000175336 (APOF), ENSG00000103569 |
| | (AQP9), ENSG00000169083 (AR), ENSG00000118520 (ARG1), |
| | ENSG00000141505 (ASGR1), ENSG00000130707 (ASS1), ENSG00000169136 |
| | (ATF5), ENSG00000114200 (BCHE), ENSG00000145692 (BHMT), |
| | ENSG00000132840 (BHMT2), ENSG00000166278 (C2), ENSG00000123838 |
| | (C4BPA), ENSG00000123843 (C4BPB), ENSG00000157131 (C8A), |
| | ENSG00000021852 (C8B), ENSG00000113600 (C9), ENSG00000129596 (CDO1), |
| | ENSG00000198848 (CES1), ENSG00000243649 (CFB), ENSG00000000971 (CFH), |
| | ENSG00000116785 (CFHR3), ENSG00000134365 (CFHR4), ENSG00000047457 |
| | (CP), ENSG00000178772 (CPN2), ENSG00000021826 (CPS1), ENSG00000140505 |
| | (CYP1A2), ENSG00000197838 (CYP2A13), ENSG00000197408 (CYP2B6), |
| | ENSG00000108242 (CYP2C18), ENSG00000165841 (CYP2C19), |
| | ENSG00000138115 (CYP2C8), ENSG00000138109 (CYP2C9), ENSG00000100197 |
| | (CYP2D6), ENSG00000130649 (CYP2E1), ENSG00000160868 (CYP3A4), |
| | ENSG00000187048 (CYP4A11), ENSG00000186115 (CYP4F2), |
| | ENSG00000186529 (CYP4F3), ENSG00000180432 (CYP8B1), ENSG00000147647 |
| | (DPYS), ENSG00000113790 (EHHADH), ENSG00000131187 (F12), |
| | ENSG00000180210 (F2), ENSG00000198734 (F5), ENSG00000101981 (F9), |
| | ENSG00000163586 (FABP1), ENSG00000165140 (FBP1), ENSG00000171564 |
| | (FGB), ENSG00000007933 (FMO3), ENSG00000131482 (G6PC), |
| | ENSG00000112964 (GHR), ENSG00000149124 (GLYAT), ENSG00000166840 |
| | (GLYATL1), ENSG00000243955 (GSTA1), ENSG00000244067 (GSTA2), |
| | ENSG00000111713 (GYS2), ENSG00000105697 (HAMP), ENSG00000101323 |
| | (HAO1), ENSG00000116882 (HAO2), ENSG00000113924 (HGD), |
| | ENSG00000134240 (HMGCS2), ENSG00000158104 (HPD), ENSG00000110169 |
| | (HPX), ENSG00000113905 (HRG), ENSG00000117594 (HSD11B1), |
| | ENSG00000170509 (HSD17B13), ENSG00000025423 (HSD17B6), |
| | ENSG00000146678 (IGFBP1), ENSG00000115457 (IGFBP2), ENSG00000055957 |
| | (ITIH1), ENSG00000162267 (ITIH3), ENSG00000164344 (KLKB1), |
| | ENSG00000113889 (KNG1), ENSG00000145826 (LECT2), ENSG00000151224 |
| | (MAT1A), ENSG00000125144 (MT1G), ENSG00000187193 (MT1X), |
| | ENSG00000138823 (MTTP), ENSG00000166741 (NNMT), ENSG00000124253 |
| | (PCK1), ENSG00000100889 (PCK2), ENSG00000179761 (PIPOX), |
| | ENSG00000005421 (PON1), ENSG00000105852 (PON3), ENSG00000116690 |
| | (PRG4), ENSG00000126231 (PROZ), ENSG00000135069 (PSAT1), |
| | ENSG00000151552 (QDPR), ENSG00000130988 (RGN), ENSG00000148965 |
| | (SAA4), ENSG00000099194 (SCD), ENSG00000135094 (SDS), ENSG00000140093 |
| | (SERPINA10), ENSG00000186910 (SERPINA11), ENSG00000123561 |
| | (SERPINA7), ENSG00000117601 (SERPINC1), ENSG00000099937 (SERPIND1), |
| | ENSG00000167711 (SERPINF2), ENSG00000100652 (SLC10A1), |
| | ENSG00000141485 (SLC13A5), ENSG00000175003 (SLC22A1), |
| | ENSG00000140284 (SLC27A2), ENSG00000083807 (SLC27A5), |
| | ENSG00000139209 (SLC38A4), ENSG00000003989 (SLC7A2), |
| | ENSG00000140263 (SORD), ENSG00000072080 (SPP2), ENSG00000105398 |
| | (SULT2A1), ENSG00000198650 (TAT), ENSG00000151790 (TDO2), |
| | ENSG00000106327 (TFR2), ENSG00000002933 (TMEM176A), ENSG00000118271 |
| | (TTR), ENSG00000109181 (UGT2B10), ENSG00000156096 (UGT2B4), |
| | ENSG00000171234 (UGT2B7), ENSG00000100024 (UPB1), ENSG00000112299 |
| | (VNN1) |
| liver sinusoidal | ENSG00000165682 (CLEC1B), ENSG00000182566 (CLEC4G), ENSG00000104938 |
| endothelial cell | (CLEC4M), ENSG00000160339 (FCN2), ENSG00000138315 (OIT3), |
| | ENSG00000189056 (RELN) |
| pancreatic | ENSG00000216921 (AC131097.2), ENSG00000162482 (AKR7A3), |
| acinar cell | ENSG00000243480 (AMY2A), ENSG00000240038 (AMY2B), ENSG00000166825 |
| | (ANPEP), ENSG00000103375 (AQP8), ENSG00000242173 (ARHGDIG), |
| | ENSG00000174672 (BRSK2), ENSG00000114529 (C3orf52), ENSG00000215704 |
| | (CELA2B), ENSG00000204140 (CLPSL1), ENSG00000141086 (CTRL), |
| | ENSG00000138161 (CUZD1), ENSG00000138798 (EGF), ENSG00000124713 |
| | (GNMT), ENSG00000149735 (GPHA2), ENSG00000138472 (GUCA1C), |
| | ENSG00000142677 (IL22RA1), ENSG00000132854 (KANK4), ENSG00000100079 |
| | (LGALS2), ENSG00000169752 (NRG4), ENSG00000185615 (PDIA2), |
| | ENSG00000010438 (PRSS3), ENSG00000168267 (PTF1A), ENSG00000143954 |
| | (REG3G), ENSG00000178828 (RNF186), ENSG00000114204 (SERPINI2), |
| | ENSG00000139540 (SLC39A5), ENSG00000149150 (SLC43A1), |
| | ENSG00000141316 (SPACA3), ENSG00000120498 (TEX11), ENSG00000178821 |
| | (TMEM52), ENSG00000197360 (ZNF98) |
| pancreatic | ENSG00000005187 (ACSM3), ENSG00000001626 (CFTR), ENSG00000146038 |
| ductal cell | (DCDC2), ENSG00000243709 (LEFTY1), ENSG00000102837 (OLFM4), |
| | ENSG00000169856 (ONECUT1), ENSG00000170927 (PKHD1), |
| | ENSG00000148735 (PLEKHS1), ENSG00000146039 (SLC17A4), |
| | ENSG00000197506 (SLC28A3), ENSG00000138079 (SLC3A1), |
| | ENSG00000080493 (SLC4A4), ENSG00000165125 (TRPV6), ENSG00000134258 |
| | (VTCN1) |
| luminal cell of | ENSG00000146205 (ANO7), ENSG00000081181 (ARG2), ENSG00000168539 |
| prostate | (CHRM1), ENSG00000120903 (CHRNA2), ENSG00000196353 (CPNE4), |
| epithelium | ENSG00000111249 (CUX2), ENSG00000109182 (CWH43), ENSG00000086205 |
| | (FOLH1), ENSG00000182256 (GABRG3), ENSG00000159184 (HOXB13), |
| | ENSG00000167749 (KLK4), ENSG00000100285 (NEFH), ENSG00000167034 |
| | (NKX3-1), ENSG00000082556 (OPRK1), ENSG00000180785 (OR51E1), |
| | ENSG00000167332 (OR51E2), ENSG00000128655 (PDE11A), ENSG00000169213 |
| | (RAB3B), ENSG00000158715 (SLC45A3), ENSG00000124664 (SPDEF), |
| | ENSG00000139865 (TTC6), ENSG00000156687 (UNC5D) |
| lung ciliated cell | ENSG00000179869 (ABCA13), ENSG00000206199 (ANKUB1), |
| | ENSG00000214215 (C12orf74), ENSG00000159588 (CCDC17), ENSG00000185860 |
| | (CCDC190), ENSG00000162004 (CCDC78), ENSG00000128536 (CDHR3), |
| | ENSG00000222046 (DCDC2B), ENSG00000197653 (DNAH10), |
| | ENSG00000174844 (DNAH12), ENSG00000197057 (DTHD1), ENSG00000203734 |
| | (ECT2L), ENSG00000179813 (FAM216B), ENSG00000153789 (FAM92B), |
| | ENSG00000203985 (LDLRAD1), ENSG00000080572 (PIH1D3), |
| | ENSG00000188817 (SNTN), ENSG00000133115 (STOML3), ENSG00000186329 |
| | (TMEM212), ENSG00000189350 (TOGARAM2), ENSG00000231738 (TSPAN19) |
| type ii pneumocyte | ENSG00000181577 (C6orf223), ENSG00000163492 (CCDC141), |
| | ENSG00000078081 (LAMP3), ENSG00000168481 (LGI3), ENSG00000169174 |
| | (PCSK9), ENSG00000168907 (PLA2G4F), ENSG00000058335 (RASGRF1), |
| | ENSG00000047936 (ROS1), ENSG00000122852 (SFTPA1), ENSG00000259803 |
| | (SLC22A31), ENSG00000156076 (WIF1) |
| extravillous | ENSG00000105246 (EBI3), ENSG00000136488 (CSH1), ENSG00000164007 |
| trophoblast | (CLDN19), ENSG00000167618 (LAIR2), ENSG00000169495 (HTRA4), |
| | ENSG00000183734 (ASCL2), ENSG00000185269 (NOTUM), ENSG00000196083 |
| | (IL1RAP), ENSG00000204632 (HLA-G), ENSG00000206538 (VGLL3) |
| syncytiotrophoblast | ENSG00000117009 (KMO), ENSG00000131037 (EPS8L1), ENSG00000137869 |
| | (CYP19A1), ENSG00000197632 (SERPINB2), ENSG00000244476 (ERVFRD-1), |
| | ENSG00000249861 (LGALS16) |
| intestinal crypt | ENSG00000114771 (AADAC), ENSG00000144820 (ADGRG7), |
| stem cell of | ENSG00000178301 (AQP11), ENSG00000136305 (CIDEB), ENSG00000073067 |
| small intestine | (CYP2W1), ENSG00000197635 (DPP4), ENSG00000096395 (MLN), |
| | ENSG00000138823 (MTTP), ENSG00000166268 (MYRFL), ENSG00000138308 |
| | (PLA2G12B), ENSG00000163817 (SLC6A20), ENSG00000204610 (TRIM15), |
| | ENSG00000122121 (XPNPEP2) |
| intestinal | ENSG00000163499 (CRYBA2), ENSG00000163497 (FEV), ENSG00000177984 |
| enteroendocrine | (LCN15), ENSG00000131096 (PYY), ENSG00000185002 (RFX6), |
| cell | ENSG00000070031 (SCT), ENSG00000036565 (SLC18A1), ENSG00000178473 |
| | (UCN3) |
| intestinal tuft | ENSG00000121690 (DEPDC7), ENSG00000214415 (GNAT3), ENSG00000188620 |
| cell | (HMX3), ENSG00000186038 (HTR3E), ENSG00000257743 (MGAM2), |
| | ENSG00000168060 (NAALADL1), ENSG00000118094 (TREH) |
| mature enterocyte | ENSG00000103375 (AQP8), ENSG00000016602 (CLCA4), ENSG00000114455 |
| | (HHLA2), ENSG00000146039 (SLC17A4), ENSG00000163959 (SLC51A), |
| | ENSG00000186198 (SLC51B), ENSG00000197165 (SULT1A2), |
| | ENSG00000182271 (TMIGD1), ENSG00000119121 (TRPM6) |
| paneth cell of | ENSG00000142959 (BEST4), ENSG00000168748 (CA7), ENSG00000183034 |
| epithelium of large | (OTOP2) |
| intestine | |
| astrocyte in normal | ENSG00000103740 (ACSBG1), ENSG00000171885 (AQP4), ENSG00000129244 |
| brain | (ATP1B2), ENSG00000164089 (ETNPPL), ENSG00000168309 (FAM107A), |
| | ENSG00000163285 (GABRG1), ENSG00000131095 (GFAP), ENSG00000179399 |
| | (GPC5), ENSG00000161509 (GRIN2C), ENSG00000165478 (HEPACAM), |
| | ENSG00000179796 (LRRC3B), ENSG00000100427 (MLC1), ENSG00000188039 |
| | (NWD1), ENSG00000146005 (PSD2), ENSG00000164188 (RANBP3L), |
| | ENSG00000111783 (RFX4), ENSG00000147509 (RGS20), ENSG00000141469 |
| | (SLC14A1), ENSG00000182902 (SLC25A18), ENSG00000148482 (SLC39A12), |
| | ENSG00000139155 (SLCO1C1), ENSG00000156076 (WIF1) |
| excitatory neuron in | ENSG00000121753 (ADGRB2), ENSG00000134343 (ANO3), ENSG00000164061 |
| normal brain | (BSN), ENSG00000063180 (CA11), ENSG00000157782 (CABP1), |
| | ENSG00000070808 (CAMK2A), ENSG00000164076 (CAMKV), |
| | ENSG00000141668 (CBLN2), ENSG00000149654 (CDH22), ENSG00000150394 |
| | (CDH8), ENSG00000176749 (CDK5R1), ENSG00000158258 (CLSTN2), |
| | ENSG00000149970 (CNKSR2), ENSG00000196353 (CPNE4), ENSG00000175874 |
| | (CREG2), ENSG00000103316 (CRYM), ENSG00000111249 (CUX2), |
| | ENSG00000165023 (DIRAS2), ENSG00000115423 (DNAH6), ENSG00000171617 |
| | (ENC1), ENSG00000127585 (FBXL16), ENSG00000172020 (GAP43), |
| | ENSG00000119125 (GDA), ENSG00000172209 (GPR22), ENSG00000152822 |
| | (GRM1), ENSG00000121905 (HPCA), ENSG00000168830 (HTR1E), |
| | ENSG00000102468 (HTR2A), ENSG00000182674 (KCNB2), ENSG00000140015 |
| | (KCNH5), ENSG00000110427 (KIAA1549L), ENSG00000171798 (KNDC1), |
| | ENSG00000156564 (LRFN2), ENSG00000180354 (MTURN), ENSG00000123119 |
| | (NECAB1), ENSG00000104722 (NEFM), ENSG00000184613 (NELL2), |
| | ENSG00000171532 (NEUROD2), ENSG00000066248 (NGEF), ENSG00000171246 |
| | (NPTX1), ENSG00000154146 (NRGN), ENSG00000174145 (NWD2), |
| | ENSG00000130558 (OLFM1), ENSG00000118733 (OLFM3), ENSG00000168490 |
| | (PHYHIP), ENSG00000130822 (PNCK), ENSG00000074211 (PPP2R2C), |
| | ENSG00000059915 (PSD), ENSG00000184672 (RALYL), ENSG00000076864 |
| | (RAP1GAP), ENSG00000136237 (RAPGEF5), ENSG00000117152 (RGS4), |
| | ENSG00000152214 (RIT2), ENSG00000108309 (RUNDC3A), ENSG00000171509 |
| | (RXFP1), ENSG00000119042 (SATB2), ENSG00000166257 (SCN3B), |
| | ENSG00000107295 (SH3GL2), ENSG00000104888 (SLC17A7), |
| | ENSG00000118160 (SLC8A2), ENSG00000187122 (SLIT1), ENSG00000145335 |
| | (SNCA), ENSG00000106089 (STX1A), ENSG00000185518 (SV2B), |
| | ENSG00000132872 (SYT4), ENSG00000011347 (SYT7), ENSG00000135426 |
| | (TESPA1), ENSG00000130477 (UNC13A), ENSG00000175267 (VWA3A), |
| | ENSG00000169064 (ZBBX) |
| inhibitory neuron in | ENSG00000004848 (ARX), ENSG00000127152 (BCL11B), ENSG00000128683 |
| normal brain | (GAD1), ENSG00000136750 (GAD2), ENSG00000198785 (GRIN3A), |
| | ENSG00000175352 (NRIP3), ENSG00000189056 (RELN), ENSG00000151812 |
| | (SLC35F4) |
| oligodendrocyte in | ENSG00000011426 (ANLN), ENSG00000108381 (ASPA), ENSG00000172508 |
| normal brain | (CARNS1), ENSG00000117266 (CDK18), ENSG00000150656 (CNDP1), |
| | ENSG00000173786 (CNP), ENSG00000184144 (CNTN2), ENSG00000136541 |
| | (ERMN), ENSG00000086205 (FOLH1), ENSG00000170775 (GPR37), |
| | ENSG00000105695 (MAG), ENSG00000197971 (MBP), ENSG00000168314 |
| | (MOBP), ENSG00000204655 (MOG), ENSG00000124920 (MYRF), |
| | ENSG00000197430 (OPALIN), ENSG00000140479 (PCSK6), ENSG00000054690 |
| | (PLEKHH1), ENSG00000123560 (PLP1), ENSG00000099194 (SCD), |
| | ENSG00000169247 (SH3TC2), ENSG00000158865 (SLC5A11), |
| | ENSG00000084453 (SLCO1A2), ENSG00000147488 (ST18), ENSG00000164124 |
| | (TMEM144) |
| oligodendrocyte | ENSG00000139352 (ASCL1), ENSG00000072182 (ASIC4), ENSG00000118322 |
| progenitor cell in | (ATP10B), ENSG00000132692 (BCAN), ENSG00000075461 (CACNG4), |
| normal brain | ENSG00000101203 (COL20A1), ENSG00000049089 (COL9A2), |
| | ENSG00000114646 (CSPG5), ENSG00000144230 (GPR17), ENSG00000169181 |
| | (GSG1L), ENSG00000150361 (KLHL1), ENSG00000187416 (LHFPL3), |
| | ENSG00000187398 (LUZP2), ENSG00000157890 (MEGF11), ENSG00000196132 |
| | (MYT1), ENSG00000101198 (NKAIN4), ENSG00000196338 (NLGN3), |
| | ENSG00000089250 (NOS1), ENSG00000184221 (OLIG1), ENSG00000205927 |
| | (OLIG2), ENSG00000203805 (PLPP4), ENSG00000148123 (PLPPR1), |
| | ENSG00000198732 (SMOC1), ENSG00000169302 (STK32A) |
| Atrial_Cardiomyocyte | ENSG00000164270 (HTR4), ENSG00000140506 (LMAN1L), ENSG00000197616 |
| | (MYH6), ENSG00000198336 (MYL4), ENSG00000120937 (NPPB), |
| | ENSG00000165899 (OTOGL), ENSG00000089225 (TBX5) |
| Ventricular_Cardiomyocyte | ENSG00000089101 (CFAP61), ENSG00000187715 (KBTBD12), |
| | ENSG00000163827 (LRRC2), ENSG00000092054 (MYH7), ENSG00000150722 |
| | (PPP1R1C), ENSG00000140986 (RPL3L) |
| bladder urothelial cell | ENSG00000177076 (ACER2), ENSG00000152785 (BMP3), ENSG00000138152 |
| | (BTBD16), ENSG00000100867 (DHRS2), ENSG00000103044 (HAS3), |
| | ENSG00000120149 (MSX2), ENSG00000142619 (PADI3), ENSG00000248485 |
| | (PCP4L1), ENSG00000158786 (PLA2G2F), ENSG00000167653 (PSCA), |
| | ENSG00000174226 (SNX31), ENSG00000134668 (SPOCD1), ENSG00000149043 |
| | (SYT8), ENSG00000137648 (TMPRSS4), ENSG00000204616 (TRIM31), |
| | ENSG00000167165 (UGT1A6), ENSG00000105668 (UPK1A), ENSG00000114638 |
| | (UPK1B), ENSG00000110375 (UPK2), ENSG00000100373 (UPK3A) |
| smooth muscle cell | ENSG00000166509 (CLEC3A), ENSG00000143867 (OSR1), ENSG00000061455 |
| | (PRDM6), ENSG00000095303 (PTGS1) |

TABLE 2
Gini coefficient ≥0.6, ≥25% of samples with normalized counts
greater than 0.50, coefficient of variation ≤1.5

| | Genes, CV ≤ 1.5 Gini ≥ 0.6 |
|---|---|
| Early primary Spermatocyte | ENSG00000164256 (PRDM9), ENSG00000177324 (BEND2), |
| | ENSG00000187268 (FAM9C) |
| Elongated Spermatid | ENSG00000125245 (GPR18), ENSG00000168454 (TXNDC2), |
| | ENSG00000170613 (FAM71B), ENSG00000175820 (CCDC168) |
| Late primary Spermatocyte | ENSG00000156509 (FBXO43), ENSG00000169679 (BUB1), |
| | ENSG00000180071 (ANKRD18A) |
| Round Spermatid | ENSG00000120051 (CFAP58), ENSG00000134249 (ADAM30), |
| | ENSG00000164398 (ACSL6), ENSG00000212710 (CTAGE1) |
| spermatogonial stem cell | ENSG00000105255 (FSD1), ENSG00000171794 (UTF1), ENSG00000187867 |
| | (PALM3), ENSG00000262874 (C19orf84) |
| Intercalated cell | ENSG00000035720 (STAP1), ENSG00000109684 (CLNK), |
| | ENSG00000188175 (HEPACAM2) |
| Podocyte | ENSG00000113578 (FGF1), ENSG00000138792 (ENPEP), |
| | ENSG00000151490 (PTPRO), ENSG00000196549 (MME) |
| Proximal tubule | ENSG00000066813 (ACSM2B), ENSG00000081479 (LRP2), |
| | ENSG00000086991 (NOX4), ENSG00000107611 (CUBN), |
| | ENSG00000116771 (AGMAT), ENSG00000125144 (MT1G), |
| | ENSG00000131183 (SLC34A1), ENSG00000132744 (ACY3), |
| | ENSG00000135069 (PSAT1), ENSG00000136872 (ALDOB), |
| | ENSG00000138030 (KHK), ENSG00000139194 (RBP5), ENSG00000145692 |
| | (BHMT), ENSG00000147647 (DPYS), ENSG00000149124 (GLYAT), |
| | ENSG00000149452 (SLC22A8), ENSG00000164237 (CMBL), |
| | ENSG00000165449 (SLC16A9), ENSG00000166825 (ANPEP), |
| | ENSG00000171234 (UGT2B7), ENSG00000171766 (GATM), |
| | ENSG00000183747 (ACSM2A), ENSG00000211445 (GPX3), |
| | ENSG00000243955 (GSTA1) |
| astrocyte in | ENSG00000100427 (MLC1), ENSG00000103740 (ACSBG1), |
| alzheimers brain | ENSG00000129244 (ATP1B2), ENSG00000131095 (GFAP), |
| | ENSG00000141469 (SLC14A1), ENSG00000168309 (FAM107A), |
| | ENSG00000179399 (GPC5) |
| excitatory neuron in | ENSG00000011347 (SYT7), ENSG00000059915 (PSD), ENSG00000063180 |
| alzheimers brain | (CA11), ENSG00000066248 (NGEF), ENSG00000074211 (PPP2R2C), |
| | ENSG00000102468 (HTR2A), ENSG00000103316 (CRYM), |
| | ENSG00000106089 (STX1A), ENSG00000108309 (RUNDC3A), |
| | ENSG00000110427 (KIAA1549L), ENSG00000115423 (DNAH6), |
| | ENSG00000119042 (SATB2), ENSG00000123119 (NECAB1), |
| | ENSG00000127585 (FBXL16), ENSG00000130558 (OLFM1), |
| | ENSG00000135426 (TESPA1), ENSG00000145335 (SNCA), |
| | ENSG00000149970 (CNKSR2), ENSG00000154146 (NRGN), |
| | ENSG00000157782 (CABP1), ENSG00000164061 (BSN), |
| | ENSG00000166257 (SCN3B), ENSG00000171509 (RXFP1), |
| | ENSG00000171617 (ENC1), ENSG00000171798 (KNDC1), |
| | ENSG00000176749 (CDK5R1), ENSG00000180354 (MTURN), |
| | ENSG00000182674 (KCNB2), ENSG00000184613 (NELL2), |
| | ENSG00000185518 (SV2B), ENSG00000187122 (SLIT1), |
| | ENSG00000196353 (CPNE4) |
| inhibitory neuron in | ENSG00000127152 (BCL11B), ENSG00000198785 (GRIN3A) |
| alzheimers brain | |
| oligodendrocyte in | ENSG00000011426 (ANLN), ENSG00000084453 (SLCO1A2), |
| alzheimers brain | ENSG00000099194 (SCD), ENSG00000105695 (MAG), ENSG00000108381 |
| | (ASPA), ENSG00000117266 (CDK18), ENSG00000124920 (MYRF), |
| | ENSG00000136541 (ERMN), ENSG00000140479 (PCSK6), |
| | ENSG00000164124 (TMEM144), ENSG00000169247 (SH3TC2), |
| | ENSG00000172508 (CARNS1), ENSG00000197971 (MBP) |
| oligodendrocyte progenitor | ENSG00000089250 (NOS1), ENSG00000184221 (OLIG1), |
| cell in alzheimers brain | ENSG00000196132 (MYT1), ENSG00000196338 (NLGN3), |
| | ENSG00000198732 (SMOC1), ENSG00000205927 (OLIG2) |
| Hepatocytes | ENSG00000000971 (CFH), ENSG00000002933 (TMEM176A), |
| | ENSG00000003989 (SLC7A2), ENSG00000005421 (PON1), |
| | ENSG00000007933 (FMO3), ENSG00000021826 (CPS1), ENSG00000025423 |
| | (HSD17B6), ENSG00000047457 (CP), ENSG00000055957 (ITIH1), |
| | ENSG00000083807 (SLC27A5), ENSG00000099194 (SCD), |
| | ENSG00000099937 (SERPIND1), ENSG00000100024 (UPB1), |
| | ENSG00000100889 (PCK2), ENSG00000101323 (HAO1), |
| | ENSG00000103569 (AQP9), ENSG00000105398 (SULT2A1), |
| | ENSG00000106327 (TFR2), ENSG00000109181 (UGT2B10), |
| | ENSG00000110169 (HPX), ENSG00000110245 (APOC3), |
| | ENSG00000111713 (GYS2), ENSG00000112299 (VNN1), |
| | ENSG00000112964 (GHR), ENSG00000113790 (EHHADH), |
| | ENSG00000113889 (KNG1), ENSG00000113905 (HRG), ENSG00000113924 |
| | (HGD), ENSG00000115457 (IGFBP2), ENSG00000116690 (PRG4), |
| | ENSG00000117601 (SERPINC1), ENSG00000118137 (APOA1), |
| | ENSG00000118271 (TTR), ENSG00000118514 (ALDH8A1), |
| | ENSG00000118520 (ARG1), ENSG00000122787 (AKR1D1), |
| | ENSG00000124253 (PCK1), ENSG00000125144 (MT1G), |
| | ENSG00000129596 (CDO1), ENSG00000130203 (APOE), |
| | ENSG00000130208 (APOC1), ENSG00000130649 (CYP2E1), |
| | ENSG00000130707 (ASS1), ENSG00000131187 (F12), ENSG00000131482 |
| | (G6PC), ENSG00000132840 (BHMT2), ENSG00000132855 (ANGPTL3), |
| | ENSG00000134240 (HMGCS2), ENSG00000135069 (PSAT1), |
| | ENSG00000135094 (SDS), ENSG00000136872 (ALDOB), |
| | ENSG00000138109 (CYP2C9), ENSG00000138115 (CYP2C8), |
| | ENSG00000138356 (AOX1), ENSG00000139209 (SLC38A4), |
| | ENSG00000140093 (SERPINA10), ENSG00000140263 (SORD), |
| | ENSG00000141485 (SLC13A5), ENSG00000141505 (ASGR1), |
| | ENSG00000144908 (ALDH1L1), ENSG00000145192 (AHSG), |
| | ENSG00000145692 (BHMT), ENSG00000147647 (DPYS), |
| | ENSG00000149124 (GLYAT), ENSG00000151224 (MAT1A), |
| | ENSG00000151552 (QDPR), ENSG00000158104 (HPD), ENSG00000160868 |
| | (CYP3A4), ENSG00000162267 (ITIH3), ENSG00000163586 (FABP1), |
| | ENSG00000165140 (FBP1), ENSG00000166278 (C2), ENSG00000166741 |
| | (NNMT), ENSG00000166840 (GLYATL1), ENSG00000167711 (SERPINF2), |
| | ENSG00000169083 (AR), ENSG00000169136 (ATF5), ENSG00000171234 |
| | (UGT2B7), ENSG00000171564 (FGB), ENSG00000172482 (AGXT), |
| | ENSG00000172955 (ADH6), ENSG00000175003 (SLC22A1), |
| | ENSG00000179761 (PIPOX), ENSG00000180432 (CYP8B1), |
| | ENSG00000183044 (ABAT), ENSG00000183549 (ACSM5), |
| | ENSG00000183747 (ACSM2A), ENSG00000186529 (CYP4F3), |
| | ENSG00000187193 (MT1X), ENSG00000187758 (ADH1A), |
| | ENSG00000196616 (ADH1B), ENSG00000198099 (ADH4), |
| | ENSG00000198650 (TAT), ENSG00000198734 (F5), ENSG00000198848 |
| | (CES1), ENSG00000214274 (ANG), ENSG00000243649 (CFB), |
| | ENSG00000243955 (GSTA1) |
| liver sinusoidal | ENSG00000104938 (CLEC4M), ENSG00000138315 (OIT3), |
| endothelial cell | ENSG00000160339 (FCN2), ENSG00000165682 (CLEC1B), |
| | ENSG00000182566 (CLEC4G), ENSG00000189056 (RELN) |
| pancreatic acinar cell | ENSG00000100079 (LGALS2), ENSG00000114529 (C3orf52), |
| | ENSG00000132854 (KANK4), ENSG00000138161 (CUZD1), |
| | ENSG00000138798 (EGF), ENSG00000149150 (SLC43A1), |
| | ENSG00000166825 (ANPEP), ENSG00000174672 (BRSK2), |
| | ENSG00000240038 (AMY2B) |
| pancreatic ductal cell | ENSG00000005187 (ACSM3), ENSG00000080493 (SLC4A4), |
| | ENSG00000102837 (OLFM4), ENSG00000138079 (SLC3A1), |
| | ENSG00000146039 (SLC17A4), ENSG00000170927 (PKHD1), |
| | ENSG00000197506 (SLC28A3), ENSG00000243709 (LEFTY1) |
| luminal cell of prostate | ENSG00000081181 (ARG2), ENSG00000100285 (NEFH), |
| epithelium | ENSG00000111249 (CUX2), ENSG00000120903 (CHRNA2), |
| | ENSG00000128655 (PDE11A), ENSG00000146205 (ANO7), |
| | ENSG00000156687 (UNC5D), ENSG00000158715 (SLC45A3), |
| | ENSG00000167034 (NKX3-1), ENSG00000168539 (CHRM1), |
| | ENSG00000169213 (RAB3B), ENSG00000180785 (OR51E1), |
| | ENSG00000182256 (GABRG3), ENSG00000196353 (CPNE4) |
| lung ciliated cell | ENSG00000128536 (CDHR3), ENSG00000159588 (CCDC17), |
| | ENSG00000162004 (CCDC78), ENSG00000174844 (DNAH12), |
| | ENSG00000179869 (ABCA13), ENSG00000189350 (TOGARAM2), |
| | ENSG00000197057 (DTHD1), ENSG00000197653 (DNAH10), |
| | ENSG00000206199 (ANKUB1) |
| type ii pneumocyte | ENSG00000058335 (RASGRF1), ENSG00000078081 (LAMP3), |
| | ENSG00000163492 (CCDC141), ENSG00000181577 (C6orf223) |
| extravillous trophoblast | ENSG00000105246 (EBI3), ENSG00000167618 (LAIR2), ENSG00000183734 |
| | (ASCL2), ENSG00000196083 (IL1RAP), ENSG00000204632 (HLA-G), |
| | ENSG00000206538 (VGLL3) |
| syncytiotrophoblast | ENSG00000117009 (KMO), ENSG00000131037 (EPS8L1), |
| | ENSG00000137869 (CYP19A1), ENSG00000244476 (ERVFRD-1) |
| intestinal crypt | ENSG00000122121 (XPNPEP2), ENSG00000136305 (CIDEB), |
| stem cell of | ENSG00000178301 (AQP11), ENSG00000197635 (DPP4), |
| small intestine | ENSG00000204610 (TRIM15) |
| intestinal tuft cell | ENSG00000168060 (NAALADL1), ENSG00000257743 (MGAM2) |
| mature enterocyte | ENSG00000119121 (TRPM6), ENSG00000146039 (SLC17A4), |
| | ENSG00000163959 (SLC51A), ENSG00000197165 (SULT1A2) |
| astrocyte in | ENSG00000100427 (MLC1), ENSG00000103740 (ACSBG1), |
| normal brain | ENSG00000129244 (ATP1B2), ENSG00000131095 (GFAP), |
| | ENSG00000141469 (SLC14A1), ENSG00000161509 (GRIN2C), |
| | ENSG00000168309 (FAM107A), ENSG00000171885 (AQP4), |
| | ENSG00000179399 (GPC5), ENSG00000188039 (NWD1) |
| excitatory neuron | ENSG00000011347 (SYT7), ENSG00000059915 (PSD), ENSG00000063180 |
| in normal brain | (CA11), ENSG00000066248 (NGEF), ENSG00000074211 (PPP2R2C), |
| | ENSG00000076864 (RAP1GAP), ENSG00000102468 (HTR2A), |
| | ENSG00000103316 (CRYM), ENSG00000104888 (SLC17A7), |
| | ENSG00000106089 (STX1A), ENSG00000108309 (RUNDC3A), |
| | ENSG00000110427 (KIAA1549L), ENSG00000111249 (CUX2), |
| | ENSG00000115423 (DNAH6), ENSG00000118160 (SLC8A2), |
| | ENSG00000118733 (OLFM3), ENSG00000119042 (SATB2), |
| | ENSG00000119125 (GDA), ENSG00000121753 (ADGRB2), |
| | ENSG00000123119 (NECAB1), ENSG00000127585 (FBXL16), |
| | ENSG00000130477 (UNC13A), ENSG00000130558 (OLFM1), |
| | ENSG00000134343 (ANO3), ENSG00000135426 (TESPA1), |
| | ENSG00000136237 (RAPGEF5), ENSG00000145335 (SNCA), |
| | ENSG00000149970 (CNKSR2), ENSG00000150394 (CDH8), |
| | ENSG00000152822 (GRM1), ENSG00000154146 (NRGN), |
| | ENSG00000156564 (LRFN2), ENSG00000157782 (CABP1), |
| | ENSG00000158258 (CLSTN2), ENSG00000164061 (BSN), |
| | ENSG00000164076 (CAMKV), ENSG00000165023 (DIRAS2), |
| | ENSG00000166257 (SCN3B), ENSG00000168490 (PHYHIP), |
| | ENSG00000171246 (NPTX1), ENSG00000171509 (RXFP1), |
| | ENSG00000171617 (ENC1), ENSG00000171798 (KNDC1), |
| | ENSG00000174145 (NWD2), ENSG00000175267 (VWA3A), |
| | ENSG00000176749 (CDK5R1), ENSG00000180354 (MTURN), |
| | ENSG00000182674 (KCNB2), ENSG00000184613 (NELL2), |
| | ENSG00000185518 (SV2B), ENSG00000187122 (SLIT1), |
| | ENSG00000196353 (CPNE4) |
| inhibitory neuron | ENSG00000127152 (BCL11B), ENSG00000128683 (GAD1), |
| in normal brain | ENSG00000175352 (NRIP3), ENSG00000189056 (RELN), |
| | ENSG00000198785 (GRIN3A) |
| oligodendrocyte | ENSG00000011426 (ANLN), ENSG00000054690 (PLEKHH1), |
| in normal brain | ENSG00000084453 (SLCO1A2), ENSG00000099194 (SCD), |
| | ENSG00000105695 (MAG), ENSG00000108381 (ASPA), ENSG00000117266 |
| | (CDK18), ENSG00000124920 (MYRF), ENSG00000140479 (PCSK6), |
| | ENSG00000164124 (TMEM144), ENSG00000168314 (MOBP), |
| | ENSG00000169247 (SH3TC2), ENSG00000172508 (CARNS1), |
| | ENSG00000173786 (CNP), ENSG00000184144 (CNTN2), |
| | ENSG00000197971 (MBP) |
| oligodendrocyte progenitor | ENSG00000049089 (COL9A2), ENSG00000072182 (ASIC4), |
| cell in normal brain | ENSG00000089250 (NOS1), ENSG00000101203 (COL20A1), |
| | ENSG00000114646 (CSPG5), ENSG00000118322 (ATP10B), |
| | ENSG00000132692 (BCAN), ENSG00000144230 (GPR17), |
| | ENSG00000157890 (MEGF11), ENSG00000169181 (GSG1L), |
| | ENSG00000184221 (OLIG1), ENSG00000196132 (MYT1), |
| | ENSG00000196338 (NLGN3), ENSG00000198732 (SMOC1), |
| | ENSG00000205927 (OLIG2) |
| Atrial_Cardiomyocyte | ENSG00000089225 (TBX5), ENSG00000164270 (HTR4), ENSG00000198336 |
| | (MYL4) |
| Ventricular_Cardiomyocyte | ENSG00000163827 (LRRC2), ENSG00000187715 (KBTBD12) |
| bladder urothelial cell | ENSG00000100373 (UPK3A), ENSG00000100867 (DHRS2), |
| | ENSG00000103044 (HAS3), ENSG00000134668 (SPOCD1), |
| | ENSG00000137648 (TMPRSS4), ENSG00000149043 (SYT8), |
| | ENSG00000177076 (ACER2) |
| smooth muscle cell | ENSG00000061455 (PRDM6), ENSG00000095303 (PTGS1), |
| | ENSG00000143867 (OSR1) |

TABLE 3
Gini coefficient ≥0.7, ≥25% of samples with normalized
counts greater than 0.5, coefficient of variance ≤1.5

| | Genes, CV ≤ 1.5 Gini ≥ 0.7 |
|---|---|
| Early primary | ENSG00000164256 (PRDM9), ENSG00000177324 (BEND2), ENSG00000187268 |
| Spermatocyte | (FAM9C) |
| Elongated | ENSG00000125245 (GPR18), ENSG00000168454 (TXNDC2), ENSG00000170613 |
| Spermatid | (FAM71B), ENSG00000175820 (CCDC168) |
| Late primary | ENSG00000156509 (FBXO43), ENSG00000169679 (BUB1) |
| Spermatocyte | |
| Round Spermatid | ENSG00000134249 (ADAM30), ENSG00000212710 (CTAGE1) |
| spermatogonial | ENSG00000171794 (UTF1), ENSG00000262874 (C19orf84) |
| stem cell | |
| Intercalated cell | ENSG00000035720 (STAP1), ENSG00000188175 (HEPACAM2) |
| Podocyte | ENSG00000138792 (ENPEP), ENSG00000196549 (MME) |
| Proximal tubule | ENSG00000066813 (ACSM2B), ENSG00000081479 (LRP2), ENSG00000086991 |
| | (NOX4), ENSG00000107611 (CUBN), ENSG00000116771 (AGMAT), |
| | ENSG00000125144 (MT1G), ENSG00000131183 (SLC34A1), ENSG00000132744 |
| | (ACY3), ENSG00000136872 (ALDOB), ENSG00000138030 (KHK), ENSG00000139194 |
| | (RBP5), ENSG00000145692 (BHMT), ENSG00000147647 (DPYS), ENSG00000149124 |
| | (GLYAT), ENSG00000149452 (SLC22A8), ENSG00000166825 (ANPEP), |
| | ENSG00000171234 (UGT2B7), ENSG00000171766 (GATM), ENSG00000183747 |
| | (ACSM2A), ENSG00000211445 (GPX3), ENSG00000243955 (GSTA1) |
| excitatory neuron | ENSG00000066248 (NGEF), ENSG00000103316 (CRYM), ENSG00000108309 |
| in alzheimers brain | (RUNDC3A), ENSG00000110427 (KIAA1549L), ENSG00000127585 (FBXL16), |
| | ENSG00000154146 (NRGN), ENSG00000157782 (CABP1), ENSG00000171509 |
| | (RXFP1), ENSG00000187122 (SLIT1) |
| oligodendrocyte in | ENSG00000084453 (SLCO1A2), ENSG00000105695 (MAG), ENSG00000124920 |
| alzheimers brain | (MYRF), ENSG00000136541 (ERMN), ENSG00000169247 (SH3TC2), |
| | ENSG00000172508 (CARNS1), ENSG00000197971 (MBP) |
| oligodendrocyte | ENSG00000184221 (OLIG1), ENSG00000196132 (MYT1), ENSG00000205927 (OLIG2) |
| progenitor cell in | |
| alzheimers brain | |
| Hepatocytes | ENSG00000005421 (PON1), ENSG00000007933 (FMO3), ENSG00000021826 (CPS1), |
| | ENSG00000025423 (HSD17B6), ENSG00000047457 (CP), ENSG00000055957 (ITIH1), |
| | ENSG00000099937 (SERPIND1), ENSG00000100024 (UPB1), ENSG00000100889 |
| | (PCK2), ENSG00000101323 (HAO1), ENSG00000103569 (AQP9), ENSG00000105398 |
| | (SULT2A1), ENSG00000106327 (TFR2), ENSG00000109181 (UGT2B10), |
| | ENSG00000110169 (HPX), ENSG00000110245 (APOC3), ENSG00000111713 (GYS2), |
| | ENSG00000112299 (VNN1), ENSG00000113889 (KNG1), ENSG00000113905 (HRG), |
| | ENSG00000113924 (HGD), ENSG00000116690 (PRG4), ENSG00000117601 |
| | (SERPINC1), ENSG00000118137 (APOA1), ENSG00000118271 (TTR), |
| | ENSG00000118514 (ALDH8A1), ENSG00000118520 (ARG1), ENSG00000122787 |
| | (AKR1D1), ENSG00000124253 (PCK1), ENSG00000125144 (MT1G), |
| | ENSG00000130208 (APOC1), ENSG00000130649 (CYP2E1), ENSG00000131187 (F12), |
| | ENSG00000131482 (G6PC), ENSG00000132840 (BHMT2), ENSG00000132855 |
| | (ANGPTL3), ENSG00000134240 (HMGCS2), ENSG00000135094 (SDS), |
| | ENSG00000136872 (ALDOB), ENSG00000138109 (CYP2C9), ENSG00000138115 |
| | (CYP2C8), ENSG00000138356 (AOX1), ENSG00000139209 (SLC38A4), |
| | ENSG00000140093 (SERPINA10), ENSG00000141485 (SLC13A5), ENSG00000141505 |
| | (ASGR1), ENSG00000145192 (AHSG), ENSG00000145692 (BHMT), |
| | ENSG00000147647 (DPYS), ENSG00000149124 (GLYAT), ENSG00000151224 |
| | (MAT1A), ENSG00000158104 (HPD), ENSG00000160868 (CYP3A4), |
| | ENSG00000162267 (ITIH3), ENSG00000163586 (FABP1), ENSG00000165140 (FBP1), |
| | ENSG00000166840 (GLYATL1), ENSG00000167711 (SERPINF2), ENSG00000169136 |
| | (ATF5), ENSG00000171234 (UGT2B7), ENSG00000171564 (FGB), ENSG00000172482 |
| | (AGXT), ENSG00000172955 (ADH6), ENSG00000175003 (SLC22A1), |
| | ENSG00000179761 (PIPOX), ENSG00000180432 (CYP8B1), ENSG00000183549 |
| | (ACSM5), ENSG00000183747 (ACSM2A), ENSG00000186529 (CYP4F3), |
| | ENSG00000187758 (ADH1A), ENSG00000196616 (ADH1B), ENSG00000198099 |
| | (ADH4), ENSG00000198650 (TAT), ENSG00000198734 (F5), ENSG00000198848 |
| | (CES1), ENSG00000214274 (ANG), ENSG00000243649 (CFB), ENSG00000243955 |
| | (GSTA1) |
| liver sinusoidal | ENSG00000104938 (CLEC4M), ENSG00000138315 (OIT3), ENSG00000160339 |
| endothelial cell | (FCN2), ENSG00000165682 (CLEC1B), ENSG00000182566 (CLEC4G) |
| pancreatic acinar | ENSG00000100079 (LGALS2), ENSG00000132854 (KANK4), ENSG00000138161 |
| cell | (CUZD1), ENSG00000138798 (EGF), ENSG00000166825 (ANPEP), ENSG00000174672 |
| | (BRSK2), ENSG00000240038 (AMY2B) |
| pancreatic ductal | ENSG00000102837 (OLFM4), ENSG00000138079 (SLC3A1), ENSG00000146039 |
| cell | (SLC17A4), ENSG00000170927 (PKHD1), ENSG00000197506 (SLC28A3), |
| | ENSG00000243709 (LEFTY1) |
| luminal cell of | ENSG00000100285 (NEFH), ENSG00000111249 (CUX2), ENSG00000120903 |
| prostate epithelium | (CHRNA2), ENSG00000146205 (ANO7), ENSG00000158715 (SLC45A3), |
| | ENSG00000167034 (NKX3-1), ENSG00000168539 (CHRM1) |
| lung ciliated cell | ENSG00000128536 (CDHR3), ENSG00000159588 (CCDC17), ENSG00000162004 |
| | (CCDC78), ENSG00000174844 (DNAH12), ENSG00000179869 (ABCA13), |
| | ENSG00000197653 (DNAH10), ENSG00000206199 (ANKUB1) |
| type ii pneumocyte | ENSG00000078081 (LAMP3), ENSG00000163492 (CCDC141), ENSG00000181577 |
| | (C6orf223) |
| extravillous | ENSG00000105246 (EBI3), ENSG00000204632 (HLA-G), ENSG00000206538 (VGLL3) |
| trophoblast | |
| syncytiotrophoblast | ENSG00000117009 (KMO), ENSG00000131037 (EPS8L1), ENSG00000137869 |
| | (CYP19A1), ENSG00000244476 (ERVFRD-1) |
| intestinal crypt | ENSG00000122121 (XPNPEP2), ENSG00000136305 (CIDEB), ENSG00000197635 |
| stem cell of small | (DPP4), ENSG00000204610 (TRIM15) |
| intestine | |
| mature enterocyte | ENSG00000146039 (SLC17A4), ENSG00000163959 (SLC51A), ENSG00000197165 |
| | (SULT1A2) |
| astrocyte in normal | ENSG00000131095 (GFAP), ENSG00000161509 (GRIN2C), ENSG00000171885 (AQP4) |
| brain | |
| excitatory neuron | ENSG00000066248 (NGEF), ENSG00000103316 (CRYM), ENSG00000104888 |
| in normal brain | (SLC17A7), ENSG00000108309 (RUNDC3A), ENSG00000110427 (KIAA1549L), |
| | ENSG00000111249 (CUX2), ENSG00000118160 (SLC8A2), ENSG00000118733 |
| | (OLFM3), ENSG00000119125 (GDA), ENSG00000127585 (FBXL16), |
| | ENSG00000134343 (ANO3), ENSG00000152822 (GRM1), ENSG00000154146 (NRGN), |
| | ENSG00000157782 (CABP1), ENSG00000164076 (CAMKV), ENSG00000165023 |
| | (DIRAS2), ENSG00000168490 (PHYHIP), ENSG00000171246 (NPTX1), |
| | ENSG00000171509 (RXFP1), ENSG00000174145 (NWD2), ENSG00000187122 (SLIT1) |
| oligodendrocyte in | ENSG00000084453 (SLCO1A2), ENSG00000105695 (MAG), ENSG00000124920 |
| normal brain | (MYRF), ENSG00000168314 (MOBP), ENSG00000169247 (SH3TC2), |
| | ENSG00000172508 (CARNS1), ENSG00000184144 (CNTN2), ENSG00000197971 |
| | (MBP) |
| oligodendrocyte | ENSG00000114646 (CSPG5), ENSG00000132692 (BCAN), ENSG00000184221 |
| progenitor cell in | (OLIG1), ENSG00000196132 (MYT1), ENSG00000205927 (OLIG2) |
| normal brain | |
| Atrial_Cardiomyocyte | ENSG00000089225 (TBX5), ENSG00000164270 (HTR4), ENSG00000198336 (MYL4) |
| bladder urothelial | ENSG00000100373 (UPK3A), ENSG00000100867 (DHRS2), ENSG00000137648 |
| cell | (TMPRSS4), ENSG00000149043 (SYT8) |
| smooth muscle cell | ENSG00000061455 (PRDM6), ENSG00000095303 (PTGS1), ENSG00000143867 (OSR1) |

TABLE 4
Gini coefficient ≥0.8, ≥25% of samples with normalized
counts greater than 0.5, coefficient of variance ≤1.5

| | Genes, CV ≤ 1.5 Gini ≥ 0.8 |
|---|---|
| Early primary | ENSG00000177324 (BEND2), ENSG00000187268 (FAM9C) |
| Spermatocyte | |
| Elongated | ENSG00000168454 (TXNDC2), ENSG00000170613 (FAM71B), ENSG00000175820 |
| Spermatid | (CCDC168) |
| Round Spermatid | ENSG00000134249 (ADAM30), ENSG00000212710 (CTAGE1) |
| spermatogonial | ENSG00000171794 (UTF1), ENSG00000262874 (C19orf84) |
| stem cell | |
| Proximal tubule | ENSG00000066813 (ACSM2B), ENSG00000107611 (CUBN), ENSG00000116771 |
| | (AGMAT), ENSG00000131183 (SLC34A1), ENSG00000136872 (ALDOB), |
| | ENSG00000139194 (RBP5), ENSG00000145692 (BHMT), ENSG00000147647 (DPYS), |
| | ENSG00000149124 (GLYAT), ENSG00000149452 (SLC22A8), ENSG00000171234 |
| | (UGT2B7), ENSG00000183747 (ACSM2A), ENSG00000243955 (GSTA1) |
| Hepatocytes | ENSG00000005421 (PON1), ENSG00000007933 (FMO3), ENSG00000021826 (CPS1), |
| | ENSG00000025423 (HSD17B6), ENSG00000047457 (CP), ENSG00000055957 (ITIH1), |
| | ENSG00000099937 (SERPIND1), ENSG00000100024 (UPB1), ENSG00000101323 |
| | (HAO1), ENSG00000103569 (AQP9), ENSG00000105398 (SULT2A1), |
| | ENSG00000106327 (TFR2), ENSG00000109181 (UGT2B10), ENSG00000110169 |
| | (HPX), ENSG00000110245 (APOC3), ENSG00000111713 (GYS2), ENSG00000112299 |
| | (VNN1), ENSG00000113889 (KNG1), ENSG00000113905 (HRG), ENSG00000113924 |
| | (HGD), ENSG00000116690 (PRG4), ENSG00000117601 (SERPINC1), |
| | ENSG00000118137 (APOA1), ENSG00000118271 (TTR), ENSG00000118514 |
| | (ALDH8A1), ENSG00000118520 (ARG1), ENSG00000122787 (AKR1D1), |
| | ENSG00000124253 (PCK1), ENSG00000130208 (APOC1), ENSG00000130649 |
| | (CYP2E1), ENSG00000131187 (F12), ENSG00000131482 (G6PC), ENSG00000132855 |
| | (ANGPTL3), ENSG00000134240 (HMGCS2), ENSG00000135094 (SDS), |
| | ENSG00000136872 (ALDOB), ENSG00000138109 (CYP2C9), ENSG00000138115 |
| | (CYP2C8), ENSG00000138356 (AOX1), ENSG00000139209 (SLC38A4), |
| | ENSG00000140093 (SERPINA10), ENSG00000141485 (SLC13A5), ENSG00000141505 |
| | (ASGR1), ENSG00000145192 (AHSG), ENSG00000145692 (BHMT), |
| | ENSG00000147647 (DPYS), ENSG00000149124 (GLYAT), ENSG00000151224 |
| | (MAT1A), ENSG00000158104 (HPD), ENSG00000160868 (CYP3A4), |
| | ENSG00000162267 (ITIH3), ENSG00000163586 (FABP1), ENSG00000166840 |
| | (GLYATL1), ENSG00000167711 (SERPINF2), ENSG00000171234 (UGT2B7), |
| | ENSG00000171564 (FGB), ENSG00000172482 (AGXT), ENSG00000172955 (ADH6), |
| | ENSG00000175003 (SLC22A1), ENSG00000179761 (PIPOX), ENSG00000180432 |
| | (CYP8B1), ENSG00000183747 (ACSM2A), ENSG00000186529 (CYP4F3), |
| | ENSG00000187758 (ADH1A), ENSG00000198099 (ADH4), ENSG00000198650 (TAT), |
| | ENSG00000198848 (CES1), ENSG00000243649 (CFB), ENSG00000243955 (GSTA1) |
| liver sinusoidal | ENSG00000104938 (CLEC4M), ENSG00000138315 (OIT3), ENSG00000160339 |
| endothelial cell | (FCN2), ENSG00000165682 (CLEC1B) |
| pancreatic acinar | ENSG00000100079 (LGALS2), ENSG00000138161 (CUZD1), ENSG00000138798 |
| cell | (EGF), ENSG00000240038 (AMY2B) |
| pancreatic ductal | ENSG00000102837 (OLFM4), ENSG00000138079 (SLC3A1), ENSG00000146039 |
| cell | (SLC17A4), ENSG00000170927 (PKHD1), ENSG00000243709 (LEFTY1) |
| luminal cell of | ENSG00000146205 (ANO7), ENSG00000167034 (NKX3-1) |
| prostate epithelium | |
| lung ciliated cell | ENSG00000159588 (CCDC17), ENSG00000174844 (DNAH12), ENSG00000206199 |
| | (ANKUB1) |
| extravillous | ENSG00000105246 (EBI3), ENSG00000204632 (HLA-G) |
| trophoblast | |
| syncytiotrophoblast | ENSG00000117009 (KMO), ENSG00000137869 (CYP19A1), ENSG00000244476 |
| | (ERVFRD-1) |
| intestinal crypt | ENSG00000122121 (XPNPEP2), ENSG00000204610 (TRIM15) |
| stem cell of small | |
| intestine | |
| mature enterocyte | ENSG00000146039 (SLC17A4), ENSG00000163959 (SLC51A) |
| excitatory neuron | ENSG00000104888 (SLC17A7), ENSG00000134343 (ANO3) |
| in normal brain | |
| oligodendrocyte in | ENSG00000168314 (MOBP), ENSG00000197971 (MBP) |
| normal brain | |
| Atrial_Cardiomyocyte | ENSG00000089225 (TBX5), ENSG00000198336 (MYL4) |
| bladder urothelial | ENSG00000100373 (UPK3A), ENSG00000100867 (DHRS2), ENSG00000149043 |
| cell | (SYT8) |

TABLE 5
Gini coefficient ≥0.9, ≥25% of samples with normalized
counts greater than 0.5, coefficient of variance ≤1.5

| | Genes, CV ≤ 1.5 Gini ≥ 0.9 |
|---|---|
| Early primary | ENSG00000177324 (BEND2), ENSG00000187268 (FAM9C) |
| Spermatocyte | |
| Elongated | ENSG00000168454 (TXNDC2), ENSG00000170613 (FAM71B), ENSG00000175820 |
| Spermatid | (CCDC168) |
| Round Spermatid | ENSG00000134249 (ADAM30), ENSG00000212710 (CTAGE1) |
| Proximal tubule | ENSG00000066813 (ACSM2B), ENSG00000131183 (SLC34A1), ENSG00000136872 |
| | (ALDOB), ENSG00000145692 (BHMT), ENSG00000147647 (DPYS), |
| | ENSG00000149124 (GLYAT), ENSG00000149452 (SLC22A8), ENSG00000171234 |
| | (UGT2B7), ENSG00000183747 (ACSM2A) |
| Hepatocytes | ENSG00000005421 (PON1), ENSG00000007933 (FMO3), ENSG00000021826 (CPS1), |
| | ENSG00000055957 (ITIH1), ENSG00000099937 (SERPIND1), ENSG00000100024 |
| | (UPB1), ENSG00000101323 (HAO1), ENSG00000105398 (SULT2A1), |
| | ENSG00000106327 (TFR2), ENSG00000109181 (UGT2B10), ENSG00000110169 (HPX), |
| | ENSG00000110245 (APOC3), ENSG00000111713 (GYS2), ENSG00000113889 (KNG1), |
| | ENSG00000113905 (HRG), ENSG00000117601 (SERPINC1), ENSG00000118137 |
| | (APOA1), ENSG00000118271 (TTR), ENSG00000118520 (ARG1), ENSG00000122787 |
| | (AKR1D1), ENSG00000124253 (PCK1), ENSG00000130649 (CYP2E1), |
| | ENSG00000131187 (F12), ENSG00000131482 (G6PC), ENSG00000132855 (ANGPTL3), |
| | ENSG00000135094 (SDS), ENSG00000136872 (ALDOB), ENSG00000138109 |
| | (CYP2C9), ENSG00000138115 (CYP2C8), ENSG00000139209 (SLC38A4), |
| | ENSG00000140093 (SERPINA10), ENSG00000141485 (SLC13A5), ENSG00000141505 |
| | (ASGR1), ENSG00000145192 (AHSG), ENSG00000145692 (BHMT), ENSG00000147647 |
| | (DPYS), ENSG00000149124 (GLYAT), ENSG00000151224 (MAT1A), |
| | ENSG00000158104 (HPD), ENSG00000160868 (CYP3A4), ENSG00000162267 (ITIH3), |
| | ENSG00000163586 (FABP1), ENSG00000166840 (GLYATL1), ENSG00000167711 |
| | (SERPINF2), ENSG00000171234 (UGT2B7), ENSG00000171564 (FGB), |
| | ENSG00000172482 (AGXT), ENSG00000172955 (ADH6), ENSG00000175003 |
| | (SLC22A1), ENSG00000180432 (CYP8B1), ENSG00000183747 (ACSM2A), |
| | ENSG00000187758 (ADH1A), ENSG00000198099 (ADH4), ENSG00000198650 (TAT) |
| liver sinusoidal | ENSG00000138315 (OIT3), ENSG00000165682 (CLEC1B) |
| endothelial cell | |
| pancreatic acinar | ENSG00000138161 (CUZD1), ENSG00000240038 (AMY2B) |
| cell | |
| pancreatic ductal | ENSG00000138079 (SLC3A1), ENSG00000170927 (PKHD1) |
| cell | |

Table 6 provides a list of genes indicative of cell types as listered therein and associated with the Alzheimer's brain.

TABLE 6
Alzheimer's brain cell type gene profiles

| | Gene list |
|---|---|
| astrocyte in | ENSG00000100427 (MLC1), ENSG00000103740 (ACSBG1), ENSG00000111783 (RFX4), |
| alzheimers | ENSG00000129244 (ATP1B2), ENSG00000131095 (GFAP), ENSG00000139155 |
| brain | (SLCO1C1), ENSG00000141469 (SLC14A1), ENSG00000146005 (PSD2), |
| | ENSG00000147509 (RGS20), ENSG00000148482 (SLC39A12), ENSG00000156076 (WIF1), |
| | ENSG00000161509 (GRIN2C), ENSG00000163285 (GABRG1), ENSG00000164089 |
| | (ETNPPL), ENSG00000165478 (HEPACAM), ENSG00000168309 (FAM107A), |
| | ENSG00000171885 (AQP4), ENSG00000179399 (GPC5), ENSG00000179796 (LRRC3B), |
| | ENSG00000182902 (SLC25A18), ENSG00000188039 (NWD1) |
| excitatory | ENSG00000011347 (SYT7), ENSG00000059915 (PSD), ENSG00000063180 (CA11), |
| neuron in | ENSG00000066248 (NGEF), ENSG00000070808 (CAMK2A), ENSG00000074211 |
| alzheimers | (PPP2R2C), ENSG00000102468 (HTR2A), ENSG00000103316 (CRYM), |
| brain | ENSG00000104722 (NEFM), ENSG00000104888 (SLC17A7), ENSG00000106089 |
| | (STX1A), ENSG00000107295 (SH3GL2), ENSG00000108309 (RUNDC3A), |
| | ENSG00000110427 (KIAA1549L), ENSG00000115423 (DNAH6), ENSG00000117152 |
| | (RGS4), ENSG00000118160 (SLC8A2), ENSG00000118733 (OLFM3), ENSG00000119042 |
| | (SATB2), ENSG00000119125 (GDA), ENSG00000121905 (HPCA), ENSG00000123119 |
| | (NECAB1), ENSG00000127585 (FBXL16), ENSG00000130558 (OLFM1), |
| | ENSG00000132872 (SYT4), ENSG00000134343 (ANO3), ENSG00000135426 (TESPA1), |
| | ENSG00000140015 (KCNH5), ENSG00000141668 (CBLN2), ENSG00000145335 (SNCA), |
| | ENSG00000149654 (CDH22), ENSG00000149970 (CNKSR2), ENSG00000150394 (CDH8), |
| | ENSG00000152822 (GRM1), ENSG00000154146 (NRGN), ENSG00000156564 (LRFN2), |
| | ENSG00000157782 (CABP1), ENSG00000158258 (CLSTN2), ENSG00000164061 (BSN), |
| | ENSG00000164076 (CAMKV), ENSG00000165023 (DIRAS2), ENSG00000166257 |
| | (SCN3B), ENSG00000168490 (PHYHIP), ENSG00000168830 (HTR1E), ENSG00000171246 |
| | (NPTX1), ENSG00000171509 (RXFP1), ENSG00000171532 (NEUROD2), |
| | ENSG00000171617 (ENC1), ENSG00000171798 (KNDC1), ENSG00000172020 (GAP43), |
| | ENSG00000174145 (NWD2), ENSG00000175874 (CREG2), ENSG00000176749 (CDK5R1), |
| | ENSG00000180354 (MTURN), ENSG00000182674 (KCNB2), ENSG00000184613 |
| | (NELL2), ENSG00000184672 (RALYL), ENSG00000185518 (SV2B), ENSG00000187122 |
| | (SLIT1), ENSG00000196353 (CPNE4) |
| inhibitory | ENSG00000004848 (ARX), ENSG00000127152 (BCL11B), ENSG00000136750 (GAD2), |
| neuron in | ENSG00000151812 (SLC35F4), ENSG00000198785 (GRIN3A) |
| alzheimers | |
| brain | |
| oligodendrocyte | ENSG00000011426 (ANLN), ENSG00000054690 (PLEKHH1), ENSG00000084453 |
| in alzheimers | (SLCO1A2), ENSG00000086205 (FOLH1), ENSG00000099194 (SCD), ENSG00000105695 |
| brain | (MAG), ENSG00000108381 (ASPA), ENSG00000117266 (CDK18), ENSG00000123560 |
| | (PLP1), ENSG00000124920 (MYRF), ENSG00000136541 (ERMN), ENSG00000140479 |
| | (PCSK6), ENSG00000147488 (ST18), ENSG00000150656 (CNDP1), ENSG00000158865 |
| | (SLC5A11), ENSG00000164124 (TMEM144), ENSG00000168314 (MOBP), |
| | ENSG00000169247 (SH3TC2), ENSG00000170775 (GPR37), ENSG00000172508 |
| | (CARNS1), ENSG00000184144 (CNTN2), ENSG00000197430 (OPALIN), |
| | ENSG00000197971 (MBP), ENSG00000204655 (MOG) |
| oligodendrocyte | ENSG00000072182 (ASIC4), ENSG00000075461 (CACNG4), ENSG00000089250 (NOS1), |
| progenitor | ENSG00000101198 (NKAIN4), ENSG00000101203 (COL20A1), ENSG00000114646 |
| cell in | (CSPG5), ENSG00000118322 (ATP10B), ENSG00000132692 (BCAN), ENSG00000139352 |
| | (ASCL1), ENSG00000144230 (GPR17), ENSG00000148123 (PLPPR1), ENSG00000150361 |
| alzheimers | (KLHL1), ENSG00000157890 (MEGF11), ENSG00000169181 (GSG1L), |
| brain | ENSG00000169302 (STK32A), ENSG00000184221 (OLIG1), ENSG00000187398 (LUZP2), |
| | ENSG00000187416 (LHFPL3), ENSG00000196132 (MYT1), ENSG00000196338 (NLGN3), |
| | ENSG00000198732 (SMOC1), ENSG00000203805 (PLPP4), ENSG00000205927 (OLIG2) |

Table 7 provides a list of genes indicative of cell types as listered therein and associated with the bladder.

TABLE 7
bladder cell type gene profiles

| | Gene list |
|---|---|
| bladder | ENSG00000177076 (ACER2), ENSG00000152785 (BMP3), ENSG00000138152 (BTBD16), |
| urothelial | ENSG00000100867 (DHRS2), ENSG00000103044 (HAS3), ENSG00000120149 (MSX2), |
| cell | ENSG00000142619 (PADI3), ENSG00000248485 (PCP4L1), ENSG00000158786 (PLA2G2F), |
| | ENSG00000167653 (PSCA), ENSG00000174226 (SNX31), ENSG00000134668 (SPOCD1), |
| | ENSG00000149043 (SYT8), ENSG00000137648 (TMPRSS4), ENSG00000204616 (TRIM31), |
| | ENSG00000167165 (UGT1A6), ENSG00000105668 (UPK1A), ENSG00000114638 (UPK1B), |
| | ENSG00000110375 (UPK2), ENSG00000100373 (UPK3A) |
| smooth | ENSG00000166509 (CLEC3A), ENSG00000143867 (OSR1), ENSG00000061455 (PRDM6), |
| muscle | ENSG00000095303 (PTGS1) |
| cell | |

Table 8 provides a list of genes indicative of cell types as listered therein and associated with the brain.

TABLE 8
normal brain cell type gene profiles

| | Gene list |
|---|---|
| astrocyte in | ENSG00000103740 (ACSBG1), ENSG00000171885 (AQP4), ENSG00000129244 |
| normal brain | (ATP1B2), ENSG00000164089 (ETNPPL), ENSG00000168309 (FAM107A), |
| | ENSG00000163285 (GABRG1), ENSG00000131095 (GFAP), ENSG00000179399 (GPC5), |
| | ENSG00000161509 (GRIN2C), ENSG00000165478 (HEPACAM), ENSG00000179796 |
| | (LRRC3B), ENSG00000100427 (MLC1), ENSG00000188039 (NWD1), ENSG00000146005 |
| | (PSD2), ENSG00000164188 (RANBP3L), ENSG00000111783 (RFX4), ENSG00000147509 |
| | (RGS20), ENSG00000141469 (SLC14A1), ENSG00000182902 (SLC25A18), |
| | ENSG00000148482 (SLC39A12), ENSG00000139155 (SLCO1C1), ENSG00000156076 |
| | (WIF1) |
| excitatory | ENSG00000121753 (ADGRB2), ENSG00000134343 (ANO3), ENSG00000164061 (BSN), |
| neuron in | ENSG00000063180 (CA11), ENSG00000157782 (CABP1), ENSG00000070808 |
| normal brain | (CAMK2A), ENSG00000164076 (CAMKV), ENSG00000141668 (CBLN2), |
| | ENSG00000149654 (CDH22), ENSG00000150394 (CDH8), ENSG00000176749 (CDK5R1), |
| | ENSG00000158258 (CLSTN2), ENSG00000149970 (CNKSR2), ENSG00000196353 |
| | (CPNE4), ENSG00000175874 (CREG2), ENSG00000103316 (CRYM), ENSG00000111249 |
| | (CUX2), ENSG00000165023 (DIRAS2), ENSG00000115423 (DNAH6), ENSG00000171617 |
| | (ENC1), ENSG00000127585 (FBXL16), ENSG00000172020 (GAP43), ENSG00000119125 |
| | (GDA), ENSG00000172209 (GPR22), ENSG00000152822 (GRM1), ENSG00000121905 |
| | (HPCA), ENSG00000168830 (HTR1E), ENSG00000102468 (HTR2A), ENSG00000182674 |
| | (KCNB2), ENSG00000140015 (KCNH5), ENSG00000110427 (KIAA1549L), |
| | ENSG00000171798 (KNDC1), ENSG00000156564 (LRFN2), ENSG00000180354 |
| | (MTURN), ENSG00000123119 (NECAB1), ENSG00000104722 (NEFM), |
| | ENSG00000184613 (NELL2), ENSG00000171532 (NEUROD2), ENSG00000066248 |
| | (NGEF), ENSG00000171246 (NPTX1), ENSG00000154146 (NRGN), ENSG00000174145 |
| | (NWD2), ENSG00000130558 (OLFM1), ENSG00000118733 (OLFM3), ENSG00000168490 |
| | (PHYHIP), ENSG00000130822 (PNCK), ENSG00000074211 (PPP2R2C), |
| | ENSG00000059915 (PSD), ENSG00000184672 (RALYL), ENSG00000076864 (RAP1GAP), |
| | ENSG00000136237 (RAPGEF5), ENSG00000117152 (RGS4), ENSG00000152214 (RIT2), |
| | ENSG00000108309 (RUNDC3A), ENSG00000171509 (RXFP1), ENSG00000119042 |
| | (SATB2), ENSG00000166257 (SCN3B), ENSG00000107295 (SH3GL2), |
| | ENSG00000104888 (SLC17A7), ENSG00000118160 (SLC8A2), ENSG00000187122 |
| | (SLIT1), ENSG00000145335 (SNCA), ENSG00000106089 (STX1A), ENSG00000185518 |
| | (SV2B), ENSG00000132872 (SYT4), ENSG00000011347 (SYT7), ENSG00000135426 |
| | (TESPA1), ENSG00000130477 (UNC13A), ENSG00000175267 (VWA3A), |
| | ENSG00000169064 (ZBBX) |
| inhibitory | ENSG00000004848 (ARX), ENSG00000127152 (BCL11B), ENSG00000128683 (GAD1), |
| neuron in | ENSG00000136750 (GAD2), ENSG00000198785 (GRIN3A), ENSG00000175352 (NRIP3), |
| normal brain | ENSG00000189056 (RELN), ENSG00000151812 (SLC35F4) |
| oligodendrocyte | ENSG00000011426 (ANLN), ENSG00000108381 (ASPA), ENSG00000172508 (CARNS1), |
| in normal brain | ENSG00000117266 (CDK18), ENSG00000150656 (CNDP1), ENSG00000173786 (CNP), |
| | ENSG00000184144 (CNTN2), ENSG00000136541 (ERMN), ENSG00000086205 (FOLH1), |
| | ENSG00000170775 (GPR37), ENSG00000105695 (MAG), ENSG00000197971 (MBP), |
| | ENSG00000168314 (MOBP), ENSG00000204655 (MOG), ENSG00000124920 (MYRF), |
| | ENSG00000197430 (OPALIN), ENSG00000140479 (PCSK6), ENSG00000054690 |
| | (PLEKHH1), ENSG00000123560 (PLP1), ENSG00000099194 (SCD), ENSG00000169247 |
| | (SH3TC2), ENSG00000158865 (SLC5A11), ENSG00000084453 (SLCO1A2), |
| | ENSG00000147488 (ST18), ENSG00000164124 (TMEM144) |
| oligodendrocyte | ENSG00000139352 (ASCL1), ENSG00000072182 (ASIC4), ENSG00000118322 (ATP10B), |
| progenitor cell | ENSG00000132692 (BCAN), ENSG00000075461 (CACNG4), ENSG00000101203 |
| in normal brain | (COL20A1), ENSG00000049089 (COL9A2), ENSG00000114646 (CSPG5), |
| | ENSG00000144230 (GPR17), ENSG00000169181 (GSG1L), ENSG00000150361 (KLHL1), |
| | ENSG00000187416 (LHFPL3), ENSG00000187398 (LUZP2), ENSG00000157890 |
| | (MEGF11), ENSG00000196132 (MYT1), ENSG00000101198 (NKAIN4), |
| | ENSG00000196338 (NLGN3), ENSG00000089250 (NOS1), ENSG00000184221 (OLIG1), |
| | ENSG00000205927 (OLIG2), ENSG00000203805 (PLPP4), ENSG00000148123 (PLPPR1), |
| | ENSG00000198732 (SMOC1), ENSG00000169302 (STK32A) |

Table 9 provides a list of genes indicative of cell types as listered therein and associated with the heart.

TABLE 9
heart cell type gene profiles

| | Gene list |
|---|---|
| Atrial | ENSG00000164270 (HTR4), ENSG00000140506 (LMAN1L), |
| Cardiomyocyte | ENSG00000197616 (MYH6), ENSG00000198336 (MYL4), ENSG00000120937 |
| | (NPPB), ENSG00000165899 (OTOGL), ENSG00000089225 (TBX5) |
| Ventricular | ENSG00000089101 (CFAP61), ENSG00000187715 (KBTBD12), |
| Cardiomyocyte | ENSG00000163827 (LRRC2), ENSG00000092054 (MYH7), |
| | ENSG00000150722 (PPP1R1C), ENSG00000140986 (RPL3L) |

Table 10 provides a list of genes indicative of cell types as listered therein and associated with the intestine.

TABLE 10
intestine cell type gene profiles

| | Gene list |
|---|---|
| intestinal | ENSG00000114771 (AADAC), ENSG00000144820 (ADGRG7), ENSG00000178301 |
| crypt stem | (AQP11), ENSG00000136305 (CIDEB), ENSG00000073067 (CYP2W1), ENSG00000197635 |
| cell of small | (DPP4), ENSG00000096395 (MLN), ENSG00000138823 (MTTP), ENSG00000166268 |
| intestine | (MYRFL), ENSG00000138308 (PLA2G12B), ENSG00000163817 (SLC6A20), |
| | ENSG00000204610 (TRIM15), ENSG00000122121 (XPNPEP2) |
| intestinal | ENSG00000163499 (CRYBA2), ENSG00000163497 (FEV), ENSG00000177984 (LCN15), |
| enteroendocrine | ENSG00000131096 (PYY), ENSG00000185002 (RFX6), ENSG00000070031 (SCT), |
| cell | ENSG00000036565 (SLC18A1), ENSG00000178473 (UCN3) |
| intestinal tuft | ENSG00000121690 (DEPDC7), ENSG00000214415 (GNAT3), ENSG00000188620 (HMX3), |
| cell | ENSG00000186038 (HTR3E), ENSG00000257743 (MGAM2), ENSG00000168060 |
| | (NAALADL1), ENSG00000118094 (TREH) |
| mature | ENSG00000103375 (AQP8), ENSG00000016602 (CLCA4), ENSG00000114455 (HHLA2), |
| enterocyte | ENSG00000146039 (SLC17A4), ENSG00000163959 (SLC51A), ENSG00000186198 |
| | (SLC51B), ENSG00000197165 (SULT1A2), ENSG00000182271 (TMIGD1), |
| | ENSG00000119121 (TRPM6) |
| paneth cell of | ENSG00000142959 (BEST4), ENSG00000168748 (CA7), ENSG00000183034 (OTOP2) |
| epithelium of | |
| large intestine | |

Table 11 provides a list of genes indicative of cell types as listered therein and associated with the kidney.

TABLE 11
kidney cell type gene profiles

| | Gene list |
|---|---|
| Intercalated | ENSG00000147614 (ATP6V0D2), ENSG00000151418 (ATP6V1G3), ENSG00000109684 |
| cell | (CLNK), ENSG00000173253 (DMRT2), ENSG00000168269 (FOXI1), ENSG00000188175 |
| | (HEPACAM2), ENSG00000113073 (SLC4A9), ENSG00000035720 (STAP1), |
| | ENSG00000143001 (TMEM61) |
| Podocyte | ENSG00000138792 (ENPEP), ENSG00000113578 (FGF1), ENSG00000196549 (MME), |
| | ENSG00000116218 (NPHS2), ENSG00000151490 (PTPRO) |
| Principal | ENSG00000150201 (FXYD4), ENSG00000160951 (PTGER1), ENSG00000110693 (SOX6) |
| cell | |
| Proximal | ENSG00000153086 (ACMSD), ENSG00000183747 (ACSM2A), ENSG00000066813 |
| tubule | (ACSM2B), ENSG00000132744 (ACY3), ENSG00000116771 (AGMAT), ENSG00000113492 |
| | (AGXT2), ENSG00000136872 (ALDOB), ENSG00000166825 (ANPEP), ENSG00000204653 |
| | (ASPDH), ENSG00000129151 (BBOX1), ENSG00000145692 (BHMT), ENSG00000164237 |
| | (CMBL), ENSG00000205279 (CTXN3), ENSG00000107611 (CUBN), ENSG00000132437 |
| | (DDC), ENSG00000015413 (DPEP1), ENSG00000147647 (DPYS), ENSG00000162391 |
| | (FAM151A), ENSG00000010932 (FMO1), ENSG00000171766 (GATM), ENSG00000149124 |
| | (GLYAT), ENSG00000211445 (GPX3), ENSG00000243955 (GSTA1), ENSG00000244067 |
| | (GSTA2), ENSG00000116882 (HAO2), ENSG00000138030 (KHK), ENSG00000081479 (LRP2), |
| | ENSG00000100253 (MIOX), ENSG00000125144 (MT1G), ENSG00000205358 (MT1H), |
| | ENSG00000144035 (NAT8), ENSG00000086991 (NOX4), ENSG00000174827 (PDZK1), |
| | ENSG00000250799 (PRODH2), ENSG00000135069 (PSAT1), ENSG00000139194 (RBP5), |
| | ENSG00000178828 (RNF186), ENSG00000081800 (SLC13A1), ENSG00000158296 |
| | (SLC13A3), ENSG00000165449 (SLC16A9), ENSG00000124564 (SLC17A3), |
| | ENSG00000197901 (SLC22A6), ENSG00000149452 (SLC22A8), ENSG00000131183 |
| | (SLC34A1), ENSG00000148942 (SLC5A12), ENSG00000137251 (TINAG), ENSG00000171234 |
| | (UGT2B7) |
| Thick | ENSG00000113946 (CLDN16), ENSG00000130829 (DUSP9), ENSG00000179399 (GPC5), |
| ascending | ENSG00000169344 (UMOD) |
| limb of | |
| Loop of | |
| Henle cell | |

Table 12 provides a list of genes indicative of cell types as listered therein and associated with the liver.

TABLE 12
liver cell type gene profiles

| | Gene list |
|---|---|
| Hepatocytes | ENSG00000121410 (A1BG), ENSG00000183044 (ABAT), ENSG00000183747 (ACSM2A), |
| | ENSG00000183549 (ACSM5), ENSG00000187758 (ADH1A), ENSG00000196616 (ADH1B), |
| | ENSG00000198099 (ADH4), ENSG00000172955 (ADH6), ENSG00000079557 (AFM), |
| | ENSG00000172482 (AGXT), ENSG00000145192 (AHSG), ENSG00000198610 (AKR1C4), |
| | ENSG00000122787 (AKR1D1), ENSG00000144908 (ALDH1L1), ENSG00000118514 |
| | (ALDH8A1), ENSG00000136872 (ALDOB), ENSG00000214274 (ANG), ENSG00000132855 |
| | (ANGPTL3), ENSG00000138356 (AOX1), ENSG00000118137 (APOA1), ENSG00000110243 |
| | (APOA5), ENSG00000130208 (APOC1), ENSG00000110245 (APOC3), ENSG00000267467 |
| | (APOC4), ENSG00000224916 (APOC4-APOC2), ENSG00000130203 (APOE), |
| | ENSG00000175336 (APOF), ENSG00000103569 (AQP9), ENSG00000169083 (AR), |
| | ENSG00000118520 (ARG1), ENSG00000141505 (ASGR1), ENSG00000130707 (ASS1), |
| | ENSG00000169136 (ATF5), ENSG00000114200 (BCHE), ENSG00000145692 (BHMT), |
| | ENSG00000132840 (BHMT2), ENSG00000166278 (C2), ENSG00000123838 (C4BPA), |
| | ENSG00000123843 (C4BPB), ENSG00000157131 (C8A), ENSG00000021852 (C8B), |
| | ENSG00000113600 (C9), ENSG00000129596 (CDO1), ENSG00000198848 (CES1), |
| | ENSG00000243649 (CFB), ENSG00000000971 (CFH), ENSG00000116785 (CFHR3), |
| | ENSG00000134365 (CFHR4), ENSG00000047457 (CP), ENSG00000178772 (CPN2), |
| | ENSG00000021826 (CPS1), ENSG00000140505 (CYP1A2), ENSG00000197838 (CYP2A13), |
| | ENSG00000197408 (CYP2B6), ENSG00000108242 (CYP2C18), ENSG00000165841 |
| | (CYP2C19), ENSG00000138115 (CYP2C8), ENSG00000138109 (CYP2C9), |
| | ENSG00000100197 (CYP2D6), ENSG00000130649 (CYP2E1), ENSG00000160868 (CYP3A4), |
| | ENSG00000187048 (CYP4A11), ENSG00000186115 (CYP4F2), ENSG00000186529 |
| | (CYP4F3), ENSG00000180432 (CYP8B1), ENSG00000147647 (DPYS), ENSG00000113790 |
| | (EHHADH), ENSG00000131187 (F12), ENSG00000180210 (F2), ENSG00000198734 (F5), |
| | ENSG00000101981 (F9), ENSG00000163586 (FABP1), ENSG00000165140 (FBP1), |
| | ENSG00000171564 (FGB), ENSG00000007933 (FMO3), ENSG00000131482 (G6PC), |
| | ENSG00000112964 (GHR), ENSG00000149124 (GLYAT), ENSG00000166840 (GLYATL1), |
| | ENSG00000243955 (GSTA1), ENSG00000244067 (GSTA2), ENSG00000111713 (GYS2), |
| | ENSG00000105697 (HAMP), ENSG00000101323 (HAO1), ENSG00000116882 (HAO2), |
| | ENSG00000113924 (HGD), ENSG00000134240 (HMGCS2), ENSG00000158104 (HPD), |
| | ENSG00000110169 (HPX), ENSG00000113905 (HRG), ENSG00000117594 (HSD11B1), |
| | ENSG00000170509 (HSD17B13), ENSG00000025423 (HSD17B6), ENSG00000146678 |
| | (IGFBP1), ENSG00000115457 (IGFBP2), ENSG00000055957 (ITIH1), ENSG00000162267 |
| | (ITIH3), ENSG00000164344 (KLKB1), ENSG00000113889 (KNG1), ENSG00000145826 |
| | (LECT2), ENSG00000151224 (MAT1A), ENSG00000125144 (MT1G), ENSG00000187193 |
| | (MT1X), ENSG00000138823 (MTTP), ENSG00000166741 (NNMT), ENSG00000124253 |
| | (PCK1), ENSG00000100889 (PCK2), ENSG00000179761 (PIPOX), ENSG00000005421 |
| | (PON1), ENSG00000105852 (PON3), ENSG00000116690 (PRG4), ENSG00000126231 |
| | (PROZ), ENSG00000135069 (PSAT1), ENSG00000151552 (QDPR), ENSG00000130988 |
| | (RGN), ENSG00000148965 (SAA4), ENSG00000099194 (SCD), ENSG00000135094 (SDS), |
| | ENSG00000140093 (SERPINA10), ENSG00000186910 (SERPINA11), ENSG00000123561 |
| | (SERPINA7), ENSG00000117601 (SERPINC1), ENSG00000099937 (SERPIND1), |
| | ENSG00000167711 (SERPINF2), ENSG00000100652 (SLC10A1), ENSG00000141485 |
| | (SLC13A5), ENSG00000175003 (SLC22A1), ENSG00000140284 (SLC27A2), |
| | ENSG00000083807 (SLC27A5), ENSG00000139209 (SLC38A4), ENSG00000003989 |
| | (SLC7A2), ENSG00000140263 (SORD), ENSG00000072080 (SPP2), ENSG00000105398 |
| | (SULT2A1), ENSG00000198650 (TAT), ENSG00000151790 (TDO2), ENSG00000106327 |
| | (TFR2), ENSG00000002933 (TMEM176A), ENSG00000118271 (TTR), ENSG00000109181 |
| | (UGT2B10), ENSG00000156096 (UGT2B4), ENSG00000171234 (UGT2B7), |
| | ENSG00000100024 (UPB1), ENSG00000112299 (VNN1) |
| Liver | ENSG00000165682 (CLEC1B), ENSG00000182566 (CLEC4G), ENSG00000104938 |
| sinusoidal | (CLEC4M), ENSG00000160339 (FCN2), ENSG00000138315 (OIT3), ENSG00000189056 |
| endothelial | (RELN) |
| cell | |

Table 13 provides a list of genes indicative of cell types as listered therein and associated with the lung.

TABLE 13
lung cell type gene profiles

| | Gene list |
|---|---|
| lung ciliated | ENSG00000179869 (ABCA13), ENSG00000206199 (ANKUB1), ENSG00000214215 |
| cell | (C12orf74), ENSG00000159588 (CCDC17), ENSG00000185860 (CCDC190), |
| | ENSG00000162004 (CCDC78), ENSG00000128536 (CDHR3), ENSG00000222046 |
| | (DCDC2B), ENSG00000197653 (DNAH10), ENSG00000174844 (DNAH12), |
| | ENSG00000197057 (DTHD1), ENSG00000203734 (ECT2L), ENSG00000179813 |
| | (FAM216B), ENSG00000153789 (FAM92B), ENSG00000203985 (LDLRAD1), |
| | ENSG00000080572 (PIH1D3), ENSG00000188817 (SNTN), ENSG00000133115 (STOML3), |
| | ENSG00000186329 (TMEM212), ENSG00000189350 (TOGARAM2), ENSG00000231738 |
| | (TSPAN19) |
| type ii | ENSG00000181577 (C6orf223), ENSG00000163492 (CCDC141), ENSG00000078081 |
| pneumocyte | (LAMP3), ENSG00000168481 (LGI3), ENSG00000169174 (PCSK9), ENSG00000168907 |
| | (PLA2G4F), ENSG00000058335 (RASGRF1), ENSG00000047936 (ROS1), |
| | ENSG00000122852 (SFTPA1), ENSG00000259803 (SLC22A31), ENSG00000156076 (WIF1) |

Table 14 provides a list of genes indicative of cell types as listered therein and associated with the pancreas.

TABLE 14
pancreas cell type gene profiles

| | Gene list |
|---|---|
| pancreatic | ENSG00000216921 (AC131097.2), ENSG00000162482 (AKR7A3), ENSG00000243480 |
| acinar cell | (AMY2A), ENSG00000240038 (AMY2B), ENSG00000166825 (ANPEP), ENSG00000103375 |
| | (AQP8), ENSG00000242173 (ARHGDIG), ENSG00000174672 (BRSK2), ENSG00000114529 |
| | (C3orf52), ENSG00000215704 (CELA2B), ENSG00000204140 (CLPSL1), ENSG00000141086 |
| | (CTRL), ENSG00000138161 (CUZD1), ENSG00000138798 (EGF), ENSG00000124713 (GNMT), |
| | ENSG00000149735 (GPHA2), ENSG00000138472 (GUCA1C), ENSG00000142677 (IL22RA1), |
| | ENSG00000132854 (KANK4), ENSG00000100079 (LGALS2), ENSG00000169752 (NRG4), |
| | ENSG00000185615 (PDIA2), ENSG00000010438 (PRSS3), ENSG00000168267 (PTF1A), |
| | ENSG00000143954 (REG3G), ENSG00000178828 (RNF186), ENSG00000114204 (SERPINI2), |
| | ENSG00000139540 (SLC39A5), ENSG00000149150 (SLC43A1), ENSG00000141316 (SPACA3), |
| | ENSG00000120498 (TEX11), ENSG00000178821 (TMEM52), ENSG00000197360 (ZNF98) |
| pancreatic | ENSG00000005187 (ACSM3), ENSG00000001626 (CFTR), ENSG00000146038 (DCDC2), |
| ductal cell | ENSG00000243709 (LEFTY1), ENSG00000102837 (OLFM4), ENSG00000169856 (ONECUT1), |
| | ENSG00000170927 (PKHD1), ENSG00000148735 (PLEKHS1), ENSG00000146039 (SLC17A4), |
| | ENSG00000197506 (SLC28A3), ENSG00000138079 (SLC3A1), ENSG00000080493 (SLC4A4), |
| | ENSG00000165125 (TRPV6), ENSG00000134258 (VTCN1) |

Table 15 provides a list of genes indicative of cell types as listered therein and associated with protate.

TABLE 15
prostate cell type gene profiles

| | Gene list |
|---|---|
| luminal | ENSG00000146205 (ANO7), ENSG00000081181 (ARG2), ENSG00000168539 (CHRM1), |
| cell of | ENSG00000120903 (CHRNA2), ENSG00000196353 (CPNE4), ENSG00000111249 (CUX2), |
| prostate | ENSG00000109182 (CWH43), ENSG00000086205 (FOLH1), ENSG00000182256 (GABRG3), |
| epithelium | ENSG00000159184 (HOXB13), ENSG00000167749 (KLK4), ENSG00000100285 (NEFH), |
| | ENSG00000167034 (NKX3-1), ENSG00000082556 (OPRK1), ENSG00000180785 (OR51E1), |
| | ENSG00000167332 (OR51E2), ENSG00000128655 (PDE11A), ENSG00000169213 (RAB3B), |
| | ENSG00000158715 (SLC45A3), ENSG00000124664 (SPDEF), ENSG00000139865 (TTC6), |
| | ENSG00000156687 (UNC5D) |

Table 16 provides a list of genes indicative of cell types as listered therein and associated with the placenta.

TABLE 16
placenta trophoblast gene profiles

| | Gene list |
|---|---|
| extravillous | ENSG00000105246 (EBI3), ENSG00000136488 (CSH1), ENSG00000164007 (CLDN19), |
| trophoblast | ENSG00000167618 (LAIR2), ENSG00000169495 (HTRA4), ENSG00000183734 |
| | (ASCL2), ENSG00000185269 (NOTUM), ENSG00000196083 (IL1RAP), |
| | ENSG00000204632 (HLA-G), ENSG00000206538 (VGLL3) |
| syncytiotrophoblast | ENSG00000117009 (KMO), ENSG00000131037 (EPS8L1), ENSG00000137869 |
| | (CYP19A1), ENSG00000197632 (SERPINB2), ENSG00000244476 (ERVFRD-1), |
| | ENSG00000249861 (LGALS16) |

Table 17 provides a list of genes indicative of cell types as listered therein and associated with the testis.

TABLE 17
testis gene profiles

| | Gene list |
|---|---|
| Early primary | ENSG00000177324 (BEND2), ENSG00000187268 (FAM9C), ENSG00000189401 |
| Spermatocyte | (OTUD6A), ENSG00000164256 (PRDM9), ENSG00000182459 (TEX19) |
| Elongated | ENSG00000183559 (C10orf120), ENSG00000173728 (Clorf100), ENSG00000125975 |
| Spermatid | (C20orf173), ENSG00000175820 (CCDC168), ENSG00000178395 (CCDC185), |
| | ENSG00000125815 (CST8), ENSG00000214866 (DCDC2C), ENSG00000172404 |
| | (DNAJB7), ENSG00000170613 (FAM71B), ENSG00000125245 (GPR18), |
| | ENSG00000214686 (IQCF6), ENSG00000205858 (LRRC72), ENSG00000165076 |
| | (PRSS37), ENSG00000258223 (PRSS58), ENSG00000181240 (SLC25A41), |
| | ENSG00000153060 (TEKT5), ENSG00000196900 (TEX43), ENSG00000124251 |
| | (TP53TG5), ENSG00000155890 (TRIM42), ENSG00000120440 (TTLL2), |
| | ENSG00000168454 (TXNDC2), ENSG00000162843 (WDR64) |
| Late primary | ENSG00000180071 (ANKRD18A), ENSG00000148513 (ANKRD30A), ENSG00000169679 |
| Spermatocyte | (BUB1), ENSG00000180116 (C12orf40), ENSG00000156509 (FBXO43), |
| | ENSG00000171989 (LDHAL6B) |
| Round | ENSG00000164398 (ACSL6), ENSG00000134249 (ADAM30), ENSG00000120051 |
| Spermatid | (CFAP58), ENSG00000212710 (CTAGE1), ENSG00000164334 (FAM170A), |
| | ENSG00000179796 (LRRC3B), ENSG00000184507 (NUTM1), ENSG00000078795 |
| | (PKD2L2) |
| spermatogonial | ENSG00000262874 (C19orf84), ENSG00000163530 (DPPA2), ENSG00000105255 (FSD1), |
| stem cell | ENSG00000187867 (PALM3), ENSG00000171794 (UTF1) |
| Sertoli Cell | ENSG00000101448 (EPPIN), ENSG00000147378 (FATE1), ENSG00000243955 (GSTA1), |
| | ENSG00000123999 (INHA), ENSG00000171864 (PRND), ENSG00000204065 (TCEAL5), |
| | ENSG00000204071 (TCEAL6) |

The status of a cell type can be determined by measuring the presence, absence or amount of cfRNA for the indicated genes. As the indicated genes are specific for the cell types, detection of cfRNA from the indicated genes, or a subset thereof, will indicate the status of the particular cell type in the human. As an example, one can select a number of the indicative genes (e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or more or all) as listed in a table above, and detect the number of cfRNA for each indicative gene. A “score” representative of the detection of cfRNA for the indicative genes can then be generated. The score can indicate presence or absence of cfRNA for the various indicative genes or can be representative of the number of copies of the cfRNA detected. In some embodiments, the number of cfRNA can be determined individually (i.e., per gene) to generate a score for each gene (for example the score could be the number of copies for a given gene). In these embodiments, each score can be compared with a control value or range for the respective gene. Alternatively, in some embodiments, the number of cfRNA can be summed to generate a single value (a single score) that can be compared to a single control value or range. For example in this case, the value generated is indifferent whether a large number of copies of one cfRNA is detected with few or no cfRNA copies of the other genes or a small number of different cfRNAs is detected because the value determined is the sum of the number of copies of all of the indicative genes assayed.

The number of cfRNA detected (whether individual or summed as discussed above) can be compared to a control value. The control value can be a calculated value, for example representative of a median or mean of healthy individuals—or diseased individuals—for the same indicative gene(s) so that a comparison between the population and the subject assayed can be determined. In other embodiments, the number of cfRNA detected from a subject can be compared over time. In other words, trends in number of cfRNA detected can be compared over time, optionally for example before and after a treatment (e.g., drug administration). Such trends over time in a subject can be used to assist selecting or changing drug dosage, or for example to measure responsiveness (positive or negative) to a treatment or toxic event experienced by the subject. In yet another embodiment, scores from two different cell types (detected by their respective cfRNAs) can be compared. Alternatively a score from a first cell type can be normalized (e.g., via a ratio) to a score from a second cell type. This can be useful in, but is not limited to, embodiments in which one cell type is of interest (possibly changing or indicating a disease state) and the second cell type is not expected to significantly change, thereby acting as a normalizing factor to compare with other data. Alternatively, both cell types can be expected to change depending on disease state but their ratio can be used.

cfRNA Detection

In order to evaluate cell type status in a human subject, cfRNA is isolated from a sample of a bodily fluid that does not contain cells, e.g., a blood sample lacking platelets and other blood cells, e.g., a serum or plasma sample, or alternatively urine, obtained from a human subject. The cfRNA is processed to detect and optionally quantify, cfRNA, e.g., corresponding to indicative genes as provided above for various cell types. In some embodiments, the sample is obtained from a human subject that is diagnosed or suspected of having a disease involving the cell type, or the human is going or about to go through a treatment (e.g., a drug treatment) and two or more samples are taken over time and compared to monitor changes in a cell type.

The level of RNA in a cfRNA sample obtained from a subject, e.g., a plasma or serum or urine sample or other sample as described herein, can be detected or measured by a variety of methods including, but not limited to, an amplification assay, sequencing assay, or a microarray chip (hybridization) assay. As used herein, “amplification” of a nucleic acid sequence has its usual meaning, and refers to in vitro techniques for enzymatically increasing the number of copies of a target sequence. Amplification methods include both asymmetric methods in which the predominant product is single-stranded and conventional methods in which the predominant product is double-stranded. The term “microarray” refers to an ordered arrangement of hybridizable elements, e.g., gene-specific oligonucleotides, attached to a substrate. Hybridization of nucleic acids from the sample to be evaluated is determined and converted to a quantitative value representing relative gene expression levels.

Non-limiting examples of methods to evaluate levels of cfRNA include amplification assays such as quantitative RT-PCR, digital PCR, massively parallel sequencing, microarray analysis; ligation chain reaction, oligonucleotide elongation assays, multiplexed assays, such as multiplexed amplification assays. In some embodiments, cfRNA presence or amount is determined by sequencing, e.g., using massively parallel sequencing methodologies. For example, RNA-Seq can be employed to determine RNA expression levels. Illustrative methods for cfRNA analysis are described, for example, in WO2019/084033.

Measured cfRNA values can be normalized to account for sample-to-sample variations in RNA isolation and the like. Methods for normalization are well known in the art. In some embodiments, the number of cfRNAs is detected via massive sequencing to a certain depth, and because different values are generated at differing sequencing depths the values are normalized to correct for differences in sequencing depth prior to comparing two values (e.g., two values from one subject from different times or between a value from a subject and a control value). In some embodiments, normalization of values is performed using trimmed mean of M values (TMM) normalization (e.g., Robinson and Oshlack, Genome Biology volume 11, Article number: R25 (2010)), e.g., when using RNA-Seq to evaluate cfRNA expression levels. In some embodiments, normalized values may be obtained using a reference level for one or more of control gene; or exogenous RNA oligonucleotides such as those provided by the External RNA Controls Consortium, or all of the assayed RNA transcripts, or a subset thereof, may also serve as reference. Other possible normalization methods can include, but are not limited to, “transcripts per million” (Wagner et al., Theory in Biosciences volume 131, pages 281-285(2012); Toden et al., Scientific Advances, 2020, Vol. 6, no. 50; Chalasani, et al., Gastrointestinal and Liver Physiology, Volume 320, Issue 4, April 2021, Pages G439-G449; Ibarra, et al., Nature Communications volume 11, Article number: 400 (2020)). A control value for normalization of RNA values can be predetermined, determined concurrently, or determined after a sample is obtained from the subject. Thus, for example, the reference control level for normalization can be evaluated in the same assay or can be a known control from one or more previous assays.

Measuring the status of cell types as described herein can be used for a variety of uses, including but not limited to providing a classification of a sample (e.g., a diagnosis, prognosis) or to indicate the potential benefits (drug efficacy) or side effects. Non-limiting examples of uses for detection of cell type status includes but is not limited to: (1) monitoring treatment response as measured by cell type, (2) monitoring disparate (two or more) cell types from a single sample, measuring drug toxicity/side effects (a drug can be efficacious and/or highly toxic) and optionally changing the drug amount or kind to a subject in response to the measurement, for example, determining whether the drug is targeting the cell type desired or whether it killing other cells.

Specific cell types as described herein can be monitored for their status (e.g., health, function, etc.) as descried herein. The following provides a non-limiting listing of specific examples of how they may be used.

In some embodiments, status of an organ or tissue is detected via detecting cfRNA for some or all of the indicative genes as described herein. In some embodiments, this provides information regarding drug toxicity/side effects. In some embodiments, one or more of the cell types described in Table 1 are detected. For example, where a drug is targeting a desired target cell type, other cells may undergo transcriptional changes and/or be killed as well. For example, a change in the signature score of a cell type the drug is targeting can occur and be compared to directionality in organ or tissue cells. Cell types that can be detected in this context can include for example hepatocytes, liver sinusoidal endothelial cells, podocyte, proximal tubule, intercalated cell, loop of Henle cell, principal cell, atrial cardiomyocyte, ventricular cardiomyocyte, lung ciliataed cell, and/or type ii pneumocyte.

In some embodiments, one or more cell type is detected to monitor for or the progression of cancer. Exemplary cell types for detection in this case, can be for example, bladder, brain, intestine, liver, lung, kidney, pancreas, prostate, testis and/or the cell type where cancer is suspected. In some embodiments, the human has cancer and is optionally treated with chemotherapy and one or more cell type is detected to monitor the effect of the chemotherapy. Exemplary cancers include but are not limited to lung and colorectal cancer. Cell types that can be detected in this context, including but not limited to treatment with or without chemotherapy, can include for example: hepatocytes, liver sinusoidal endothelial cells, all renal cell types (podocyte, proximal tubule, intercalated cell, loop of Henle cell, principal cell), cardiomyocytes (atrial cardiomyocyte, ventricular cardiomyocyte), lung ciliated cell, and/or type ii pneumocyte. In some embodiments, drug toxicity is measured alongside the tumor response to treatment.

In some embodiments, one or more cell type is detected to monitor for or the progression of chronic kidney disease (CKD). In some embodiments, one or more cell type is detected to monitor for or the progression of chronic kidney disease can include for example podocyte, proximal tubule, principal cell, intercalated cell, and/or loop of Henle cell. CKD, and the progression thereof, is, in some embodiments, associated with and/or caused by type 1 or type 2 diabetes, high blood pressure, glomerulonephritis, interstitial nephritis, and/or polycystic kidney disease. In another embodiment, an aforementioned method is provided further comprising detecting serum creatinine, urine creatinine, urine protein, cystatin C, albuminuria, and/or glomerular filtration rate (GFR) and/or estimated glomerular filtration rate (eGFR) in the human.

In some embodiments, one or more cell type is detected to monitor for or the progression of minimal change disease. In some embodiments, one or more cell type detected is podocyte and other cell types implicated in protein filtration in kidney, as well as T cells. In another embodiment, an aforementioned method is provided further comprising detecting serum creatinine, urine creatinine, urine protein, cystatin C, albuminuria, and/or glomerular filtration rate (GFR) and/or estimated glomerular filtration rate (eGFR) in the human.

In some embodiments, one or more cell type is detected to monitor for or the progression of Acute Kidney Injury (AKI) and/or respective subtypes. In some embodiments, one or more cell type detected is podocyte (glomerular damage), vascular endothelial cells (vascular damage), or tubule cells (interstitial damage). Other cell types that can be detected include, e.g., tubular cell types (proximal tubule, intercalated cell, loop of Henle cell), podocyte, and/or vascular endothelial cells. AKI, and the progression thereof, is, in some embodiments, associated with and/or caused by heart failure, liver failure, sepsis, blood vessel inflammation/blockage, renal ischaemia, nephrotoxic agents, tubulointerstitial disease, glomerulonephritis, diabetes, intrarenal inflammation, and/or systemic inflammation. In another embodiment, an aforementioned method is provided further comprising detecting serum creatinine, urine creatinine, urine protein, cystatin C, albuminuria, and/or glomerular filtration rate (GFR) and/or estimated glomerular filtration rate (eGFR) in the human.

In some embodiments, one or more cell type is detected to monitor for or the progression of tubulointerstitial disease. In some embodiments, one or more cell type detected is the proximal tubule, intercalated cell, Thick ascending limb of Loop of Henle cell, and/or principal cell. In another embodiment, an aforementioned method is provided further comprising detecting serum creatinine, urine creatinine, urine protein, cystatin C, albuminuria, and/or glomerular filtration rate (GFR) and/or estimated glomerular filtration rate (eGFR) in the human.

In some embodiments, one or more cell type is detected to monitor for or the progression of obstructive nephropathy. In some embodiments, one or more cell type detected is the proximal tubule, intercalated cell, Thick ascending limb of Loop of Henle cell, and/or principal cell. In another embodiment, an aforementioned method is provided further comprising detecting serum creatinine, urine creatinine, urine protein, cystatin C, albuminuria, and/or glomerular filtration rate (GFR) and/or estimated glomerular filtration rate (eGFR) in the human.

In some embodiments, one or more cell type is detected to monitor for or the progression of inflammatory liver disease. In some embodiments, one or more cell type detected is liver sinusoidal endothelial cells, hepatocytes, leukocytes (monocyte, neutrophil), and/or lymphocytes (e.g., B or T cell).

In some embodiments, one or more cell type is detected to monitor for or the progression of glioblastoma (brain cancer). In some embodiments, one or more cell type detected is a brain cell type.

In some embodiments, one or more cell type is detected to monitor for or the progression of vaccine response. In some embodiments, one or more cell type detected is an immune cell type.

In some embodiments, one or more cell type is detected to monitor for or the progression of placental arterial invasion in remodeling. In some embodiments, one or more cell type detected is an extravillous trophoblast.

In some embodiments, one or more cell type is detected to monitor for or the progression of fertility. In some embodiments, one or more cell type detected is a testicular cell type.

In some embodiments, one or more cell type is detected to monitor for or the progression of Crohn's disease/leaky gut. In some embodiments, one or more cell type detected is intestinal epithelia and/or lymphocytes.

In some embodiments, one or more cell type is detected to monitor for or the progression of cardiac hypertrophy/remodeling. In some embodiments, one or more cell type detected is atrial cardiomyocyte and/or ventricular cardiomyocyte.

In some embodiments, one or more cell type is detected to monitor for or the progression of Parkinson's disease. In some embodiments, one or more cell type detected is a brain cell type.

The ability to non-invasively resolve cell type signatures in plasma cfRNA will both enhance existing clinical knowledge and enable increased resolution in monitoring disease progression and drug response.

In some embodiments, the drug is an immunotherapy or chemotherapeutic agent. In some embodiments, one or more cell type is detected is one that belongs to the lung (e.g. type ii pneumocyte, lung ciliated cell), the intestine (e.g. intestinal crypt stem cell of the small intestine, intestinal enteroendocrine cell, intestinal tuft cell, mature enterocyte, Paneth cell of epithelium of large intestine), and/or the heart (e.g. atrial cardiomyocyte, ventricular cardiomyocyte) and/or is involved in drug metabolism (e.g. kidney cell type or liver cell type, such as those described and included in various tables herein).

In some embodiments, one or more cell type is detected to monitor for disease progression. This includes but is not limited to cell types implicated in the disease, are targeted by a therapeutic drug, are known to respond to a disease-implicated cell type, or do not change in response to disease. In some embodiments, a given cell type is normalized by the signature score of another cell type. In some embodiments, the normalizing cell type may be independent of the numerator (e.g not expected to respond to the changing cell type). In other embodiments, the normalizing cell type may be related to the numerator (e.g. expected to change).

In some embodiments, one or more cell type is detected to stratify participants in a pharmaceutical clinical trial. In some embodiments, this provides information regarding disease subtypes that would otherwise be inaccessible (e.g. excitatory neuron, inhibitory neuron, oligodendrocyte, and/or oligodendrocyte precursor cell) and/or where invasive biopsy information is not available (e.g., Tables 1-5).

In some embodiments, one or more cell type, for example a cell type described in any one of Tables 1-5 and/or Table 7) is detected (e.g., an indicative gene(s) that is associated with the cell type is detected) to diagnose or monitor for or the progression of bladder urothelial cancer. In some embodiments, the one or more cell type detected is a bladder urothelial cell, the cell type in which this disease occurs^{6,7}. In other embodiments, one or more cell type detected is an unintended off-target of the prescribed therapeutic drug. In some embodiments, the biofluid measured is plasma or urine.

In some embodiments, one or more cell type, for example a cell type described in any one of Tables 1-5 and/or Table 8 is detected (e.g., an indicative gene(s) that is associated with the cell type is detected to diagnose or monitor for or the progression of Parkinson's disease or response to a therapeutic drug. In some embodiments, the one or more cell type detected is a brain cell type. In other embodiments, the one or more cell type detected that is implicated in Parkinson's etiology⁸ is the oligodendrocyte, the excitatory neuron, and/or the inhibitory neuron. In some embodiments, one or more cell type, for example a cell type described in any one of Tables 1-5 and/or Table 8 is detected (e.g., an indicative gene(s) that is associated with the cell type is detected) to diagnose or monitor for or the progression of glioblastoma (brain cancer). In some embodiments, the one or more cell type detected is a brain cell type. In other embodiments the one or more cell type detected is a glial cell in which the majority of this cancer case occurs, including for example, astrocyte, oligodendrocyte, and/or oligodendrocyte precursor celltypes⁹.

In some embodiments, one or more cell type, for example a cell type described in any one of Tables 1-5 and/or Table 6 and 8 is detected (e.g., an indicative gene(s) that is associated with the cell type is detected) to diagnose or monitor for or the progression of Alzheimers's disease or response to a therapeutic drug. In some embodiments, the one or more cell type detected in a brain cell type. In other embodiments, the one or more cell type detected is a neuron cell type (excitatory neuron or inhibitory neuron) and/or a glial cell (astrocyte, oligodendrocyte, oligodendrocyte precursor cell), all of which exhibit distinct cell-type specific transcriptional changes at the single cell transcriptomic level at the early stage of the disease¹⁰. In other embodiments, the one or more cell type detected is a kidney, liver, lung, heart, and/or intestine cell type (indicated in the respective tables, e.g., Tables 6-16).

In some embodiments, the biofluid measured is plasma or cerebrospinal fluid.

In some embodiments, one or more cell type, for example a cell type described in any one of Tables 1-5 and/or Table 9) is detected (e.g., an indicative gene(s) that is associated with the cell type is detected) to monitor for or the progression of cardiac hypertrophy and/or cardiac remodeling. In some embodiments, one or more cell type detected is atrial cardiomyocyte and/or ventricular cardiomyocyte^{11,12}.

In some embodiments one or more cell type, for example a cell type described in any one of Tables 1-5 and/or Table 9 is detected (e.g., an indicative gene(s) that is associated with the cell type is detected) to monitor for or the progression of heart health or function, ischemic cardiomyopathy, non-ischemic cardiomyopathy (including but not limited to infiltrative, inherited familial cardiomyopathies, amyloid cardiomyopathies, exogenous toxin induced cardiomyopathies (e.g. alcohol or chemotherapy), valvular cardiomyopathies), cardiac tumors (e.g. atrial myxoma), and/or reversible cardiomyopathies (e.g. tachycardia-induced cardiomyopathy)^{13,14}. In some embodiments, the measured cell type is atrial cardiomyocyte and/or ventricular cardiomyocyte (Tables 1-5 and/or Table 9).

In some embodiments, detection for or the progression of the aforementioned cardiomyopathies via noninvasive cell type (atrial cardiomyocyte and/or ventricular cardiomyocyte) monitoring for the early diagnosis of atrial arrhythmias (atrial fibrillation, atrial stand still, sinus arrest, and/or sinus node dysfunction) and/or ventricular arrhythmias (ventricular tachycardia, monomorphic and/or polymorphic ventricular tachycardia)¹⁵ (Tables 1-5 and/or Table 9).

In some embodiments, one or more cell type, for example a cell type described in any one of Tables 1-5 and/or Table 9 is detected (e.g., an indicative gene(s) that is associated with the cell type is detected for or the progression of heart failure. In some embodiments, the one or more cell type detected is atrial cardiomyocyte and/or ventricular cardiomyocyte¹.

In some embodiments, one or more cell type, for example a cell type described in any one of Tables 1-5 and/or Table 10 is detected (e.g., an indicative gene(s) that is associated with the cell type is detected) to diagnose or monitor for or the progression of Celiac disease. In some embodiments, the one or more cell type detected is an intestinal cell type. In other embodiments the one or more cell type detected is an intestinal crypt stem cell16, enteroendocrine cell¹⁷, enterocyte18, and/or Paneth cell¹⁶. In other embodiments the one or more cell type detected is an immune cell, such as a T cell and/or other lymphocyte.

In some embodiments, one or more cell type, for example a cell type described in any one of Tables 1-5 and/or Table 10 is detected (e.g., an indicative gene(s) that is associated with the cell type is detected) to diagnose or monitor for or the progression of Chron's disease and/or Inflammatory Bowel Disease. In some embodiments, the one or more cell type detected is an intestinal cell type. In other embodiments the one or more cell type detected is a Paneth cell¹⁹, an enterocyte²⁰, enteroendocrine cell²¹, intestinal crypt stem cell²², and/or immune cell types (T cell, NK cell, mast cell, dendritic cell, and/or neutrophils)²¹.

In some embodiments, one or more cell type, for example a cell type described in any one of Tables 1-5 and/or Table 10 is detected (e.g., an indicative gene(s) that is associated with the cell type is detected) to diagnose or monitor for or the progression of colorectal cancer. In some embodiments, the one or more cell type detected is an intestinal cell type. In other embodiments the one or more cell type detected is an intestinal crypt stem cell²³, enteroendocrine cell, intestinal tuft cell, mature enterocyte²³, and/or Paneth cell²⁴. For intestinal diseases, the biofluids assayed are plasma and/or urine in some embodiments.

In some embodiments, one or more cell type, for example a cell type described in any one of Tables 1-5 and/or Table 11 is detected (e.g., an indicative gene(s) that is associated with the cell type is detected) to monitor for or the progression of kidney cancer. In some embodiments, the one or more cell type is detected to monitor for or the progression of kidney cancer can include for example podocyte and/or tubule cells (proximal tubule intercalated cell and/or loop of Henle cell).

In some embodiments, the indicative genes for the proximal tubule include one or more of ENSG00000136872 (ALDOB), ENSG00000107611 (CUBN), ENSG00000081479 (LRP2), ENSG00000131183 (SLC34A1) and/or ENSG00000140675 (SLC5A2).

In some embodiments, one or more cell type, for example a cell type described in any one of Tables 1-5 and/or Table 12 is detected (e.g., an indicative gene(s) that is associated with the cell type is detected) to diagnose or monitor for or the progression of non-alcoholic fatty liver disease or non-alcoholic steatohepatitis. In other embodiments, the one or more cell type detected is a hepatocyte²⁵ or liver sinusoidal endothelial cell²⁶.

In some embodiments, one or more cell type, for example a cell type described in any one of Tables 1-5 and/or Table 12 is detected (e.g., an indicative gene(s) that is associated with the cell type is detected) to monitor for drug metabolism. In some embodiments, the one or more cell type detected is a liver cell type. In other embodiment, the one or more cell type detected is hepatocyte²⁵ or liver sinusoidal endothelial cell²⁷. In some embodiments, the drug is hepatically cleared.

In some embodiments, one or more cell type, for example a cell type described in any one of Tables 1-5 and/or Table 12 is detected (e.g., an indicative gene(s) that is associated with the cell type is detected) to diagnose or monitor for or the progression of liver cancer. In some embodiments, the one or more cell type detected is a liver cell type. In other embodiment, the one or more cell type detected is a hepatocyte²⁸ and/or liver sinusoidal endothelial cell²⁹. In some embodiments, for all intestinal diseases, the biofluids assayed are plasma and/or urine.

In some embodiments, one or more cell type, for example a cell type described in any one of Tables 1-5 and/or Table 13 is detected (e.g., an indicative gene(s) that is associated with the cell type is detected) to diagnose or monitor for or the progression of lung injury. In some embodiments, the one or more cell type detected is a type ii pneumocyte^{30,31}. In some embodiments, the one or more cell type detected is a lung ciliated cell³².

In some embodiments, one or more cell type, for example a cell type described in any one of Tables 1-5 and/or Table 13 is detected (e.g., an indicative gene(s) that is associated with the cell type is detected) to diagnose or monitor or the progression of lung cancer. In some embodiments the one or more cell type detected is a type ii pneumocyte³³ and/or lung ciliated cell³².

In some embodiments, one or more cell type, for example a cell type described in any one of Tables 1-5 and/or Table 11 is detected (e.g., an indicative gene(s) that is associated with the cell type is detected) to monitor for or the progression of chronic kidney disease. In some embodiments, the one or more cell type is detected to monitor for or the progression of chronic kidney disease can include for example podocyte³⁴ and/or tubule cells (proximal tubule intercalated cell, and/or loop of Henle cell)³⁵. In some embodiments a fibroblast cell type or markers are considered for normalization³⁵.

In some embodiments, one or more cell type, for example a cell type described in any one of Tables 1-5 and/or Table 14 is detected (e.g., an indicative gene(s) that is associated with the cell type is detected) to diagnose or monitor for or the progression of pancreatic cancer. In some embodiments, one or more cell type detected is a pancreatic acinar cell and/or a pancreatic ductal cell⁴⁰.

In some embodiments, one or more cell type, for example a cell type described in any one of Tables 1-5 and/or Table 14 is detected (e.g., an indicative gene(s) that is associated with the cell type is detected) to diagnose or monitor for or the progression of type i/type ii diabetes. In some embodiments, the one or more cell type detected is a pancreatic acinar cell and/or a pancreatic ductal cell.

In some embodiments, one or more cell type, for example a cell type described in any one of Tables 1-5 and/or Table 15 is detected (e.g., an indicative gene(s) that is associated with the cell type is detected) to diagnose or monitor for or the progression of prostate cancer or response to prostate cancer drug treatment. In some embodiments, the one or more cell type detected is a prostate epithelial cell⁴¹ and/or immune cell type.

In some embodiments, the biofluid is urine, semen, and/or plasma.

In some embodiments, one or more cell type, for example a cell type described in any one of Tables 1-5 and/or Table 16 is detected (e.g., an indicative gene(s) that is associated with the cell type is detected) to monitor for or the progression of fertility.

In some embodiments, one or more cell type, for example a cell type described in any one of Tables 1-5 and/or Table 17) is detected (e.g., an indicative gene(s) that is associated with the cell type is detected) to monitor for or the progression of testicular cancer. In some embodiments, the one or more cell type detected is a testicular germ cell type (early primary//late primary spermatocyte, elongated/round spermatid, spermatogonial stem cell) and/or a Sertoli cell⁴².

In some embodiments, for example relating to fertility and testicular cancer, the biofluid is plasma, urine, and/or seminal bodyfluid. Extracellular RNA has been observed in seminal bodyfluid⁴³.

### Computer Implementation

In some embodiments, a database comprising reference values for cfRNA levels of the an indicative gene set as described herein, or subset thereof, is provided. In some embodiments, a database comprising expression data from a plurality of humans, e.g. healthy humans or diseased humans, is provided. Accordingly, aspects of the disclosure provide systems and methods for the use and development of one or more database, for example to compare to a value as described herein from a human subject.

In some embodiments, a non-transitory computer-readable storage device is provided that stores computer-executable instructions that, in response to execution, cause a processor to perform operations such as one or more of those described herein. In some embodiments, the instructions can comprise comparing sequencing reads (e.g., from RNA-Seq) to a data base to identify and in some embodiments quantify cfRNAs corresponding to a number of the indicative genes of the Tables provided herein. Comparisons of sequencing reads can be implemented with sequence comparison algorithm, for example but not limited to BLAST.

In some embodiments, the instructions can include one or more of: receiving data indicating presence, absence or quantity of cell-free RNA (cfRNA) from at least 3, 4, 5, 6, 7, 8, 9, 10, 11, 12, 13, 14, 15, or more or all indicative genes for a cell type, wherein indicative genes are selected from any one of Tables 1, 2, 3, 4, or 5; generating a score based on detection of the cfRNA from the indicative genes; comparing the score to a control value or a prior score from an earlier-obtained biological sample or from a score for a different cell type from the human; upon determining that the score is above or below the control value or prior score, generating a classification of disease or prognosis of the human related to the cell type; and/or displaying the classification

Methods described herein, or parts thereof, can be implemented using a computer-based system. As used herein, “a computer-based system” refers to the hardware means, software means, and data storage means used to analyze the information obtain from a human, e.g., to compare to a control value or one or more other values obtained from an earlier or later sample from the human. The minimum hardware of the computer-based systems can comprise for example a central processing unit (CPU), input means, output means, and data storage means. Any of the currently available computer-based system are suitable for use in the present methods and systems. The data storage means may comprise any manufacture comprising data as described herein, or a memory access means that can access such a manufacture.

Any of the computer systems mentioned herein may utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

A computer system can include a plurality of the same components or subsystems, e.g., connected together by external interface, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g. an application specific integrated circuit or field programmable gate array) and/or using computer software with a generally programmable processor in a modular or integrated manner. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python or R using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

The databases may be provided in a variety of forms or media to facilitate their use. “Media” refers to a manufacture that contains the expression information of the present invention. The databases of the present invention can be recorded on computer readable media, e.g. any medium that can be read and accessed directly by a computer (e.g., an internet database). Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. Any of the presently known computer readable media can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

### Examples

Cell-free RNA (cfRNA) represents a mixture of transcripts reflecting the health status of multiple tissues3, thereby affording broad clinical utility. Existing applications span oncology and bone marrow transplantation^{1,44}, obstetrics^{3,45,46}, neurodegeneration⁴ and liver disease⁵. However, several aspects about the physiologic origins of cfRNA, including the contributing cell types of origin, remain unknown, and current assays focus on tissue-level contributions at best^{3-5,44,45}. Incorporating knowledge from cellular pathophysiology, which often forms the basis of disease6, into a liquid biopsy would more closely match the resolution afforded by invasive procedures. Single cell transcriptomics (scRNA-seq) enable insight into the heterogeneous cellular transcriptional landscapes of tissues in health and disease⁴⁷. Numerous scRNA-seq tissue atlases provide powerful reference data for defining cell type specific gene profiles in the context of an individual tissue. However, the starting set of cell types influences a differential expression analysis, which guides the assignment of a gene as cell type specific.

cfRNA originates from cell types across the human body. Therefore, interpreting a measured gene in cfRNA as cell type specific relies on the completeness of relevant atlases. The Tabula Sapiens (TSP) cell atlas⁴⁸ from 24 tissues enables the most comprehensive derivation of cell type specific gene profiles in the context of a single individual to date, all determined with uniform methods and sequencing, and this resource was used to computationally deconvolve the landscape of healthy cell type signal in healthy donor plasma. For cell types originating from tissues absent from the draft TSP atlas, specific gene profiles were derived by combining a single tissue cell atlas with comprehensive bulk transcriptomic datasets, including the Genotype-Tissue Expression (GTEx) project⁴⁹ and the Human Protein Atlas (HPA)⁵⁰.

The present disclosure defines cell type specific gene profiles in the context of the whole body to identify the cell types comprising the cf-transcriptome. First, the cell types-of-origin in the healthy human cf-transcriptome were computationally deconvolved using the TSP cell atlas. Next, striking cfRNA changes associated with cell types implicated were measured in a variety of diseases that are consistent with observed clinical pathology. Altogether, the present disclosure demonstrates that it is possible to decompose the cf-transcriptome into distinct cell type contributions even in the absence of a complete whole body single cell reference, and demonstrate that cell type specific changes in disease can be measured noninvasively using cfRNA.

### Results

### Deconvolution of Cell Type Specific Signals in the Healthy Cell Free Transcriptome

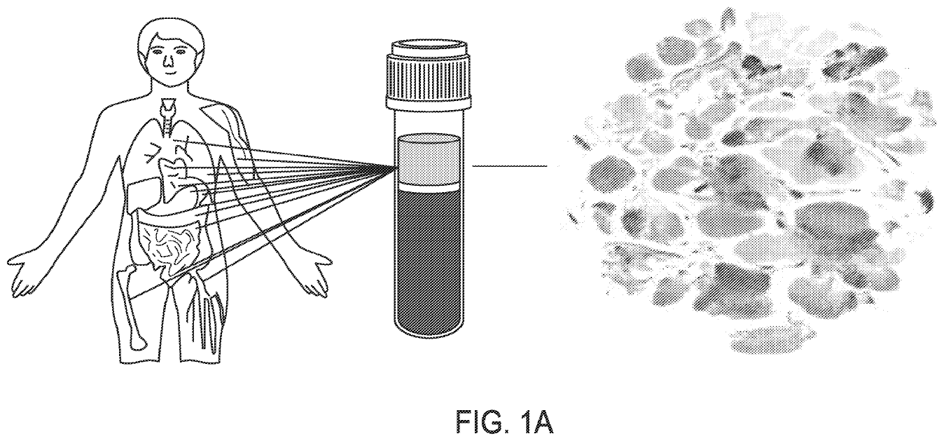

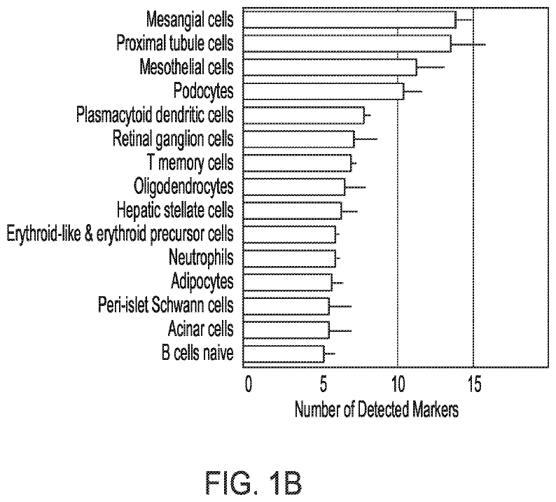

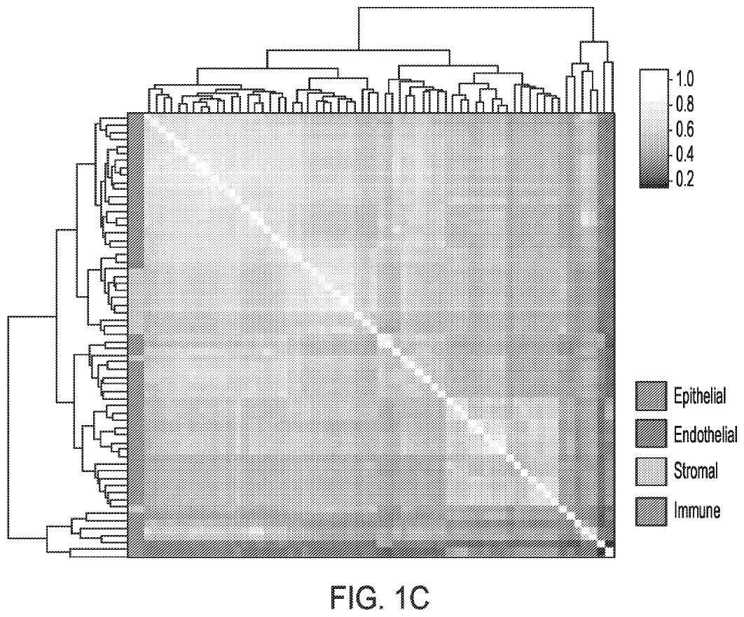

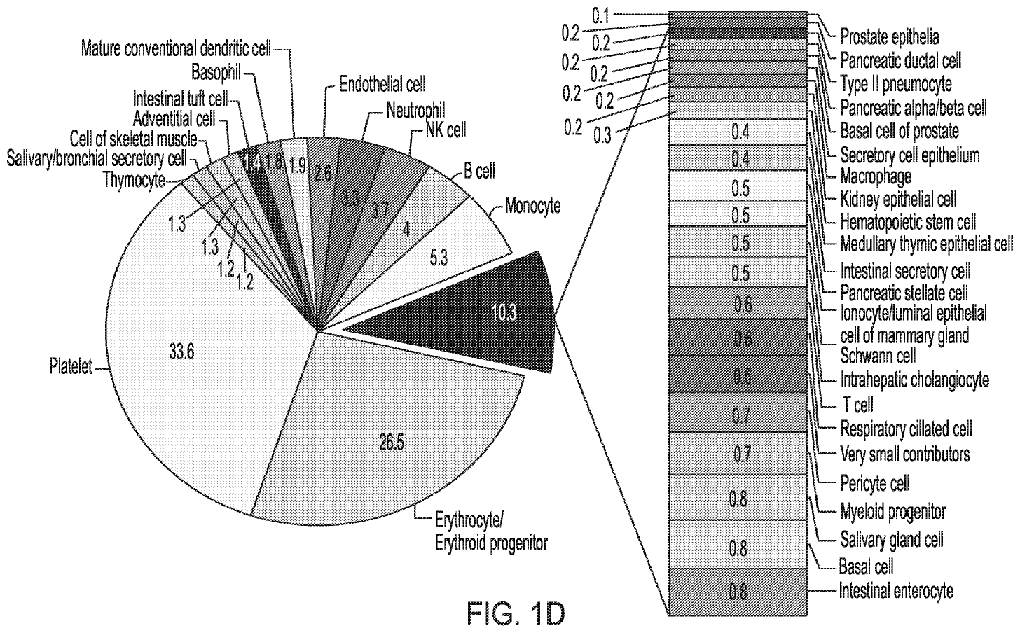

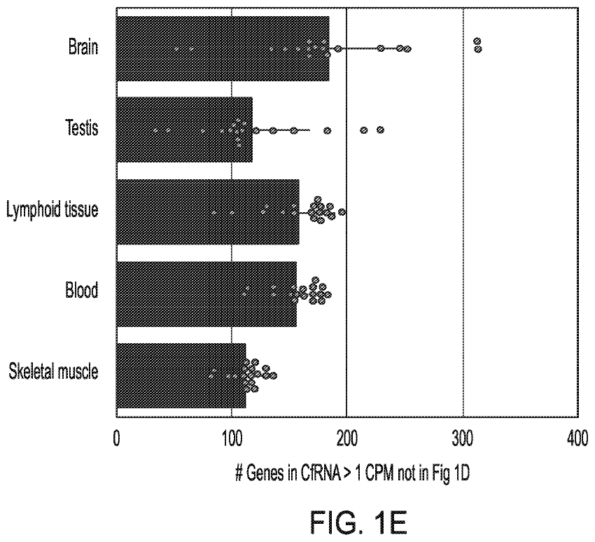

Published exome-enriched cf-transcriptome data⁴ was used to characterize the landscape of cell type specific signal in the plasma of healthy individuals (FIG. 1a). After removing low-quality cfRNA samples (Fig. S1 and Methods), the set of genes detected in healthy individuals (n=75) were intersected with a database of cell-type specific markers defined in context of the whole body⁵¹ (FIG. 1b). Marker genes for cell types originating from the blood, brain, and liver were readily detected, as previously observed at tissue level^{3-5,44,45} Kidney, GI track, and pancreas cell type markers were additionally detected (FIG. 1b).

Given the robust detection of several cell types contributing to the cf-transcriptome, the fractions of cell-type specific RNA were deconvolved using TSP. The cf-transcriptome was defined as a linear combination of cell type specific RNA contributions using a deconvolution method, nu-SVR, originally developed to decompose bulk tissue transcriptomes into fractional cell type contributions^{52,53}.

TSP 1.0 48, a multiple-donor whole-body cell atlas spanning 24 tissues and organs, was used to define a basis matrix whose gene set accurately and simultaneously resolved the distinct cell types in TSP. The basis matrix was defined using the gene space that maximized linear independence of the cell types and does not include the whole transcriptome but rather the minimum discriminatory gene set to distinguish between the cell types in TSP.

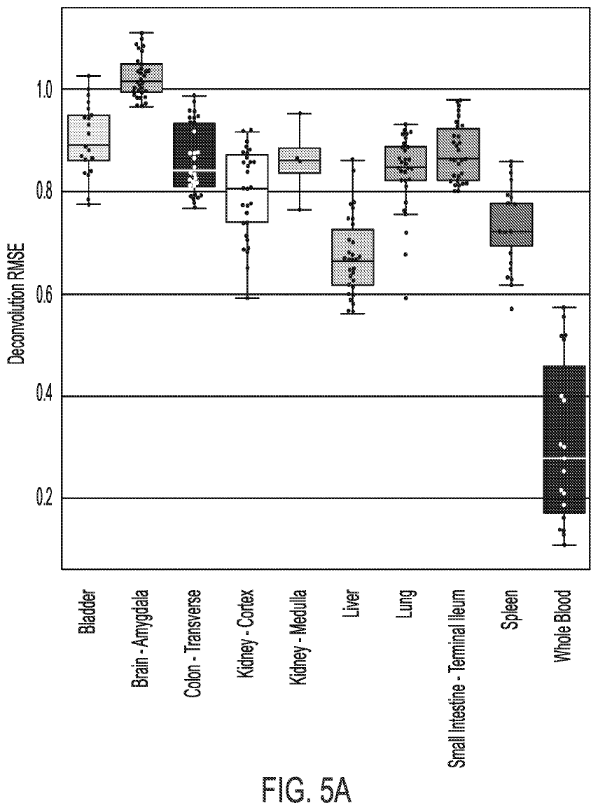

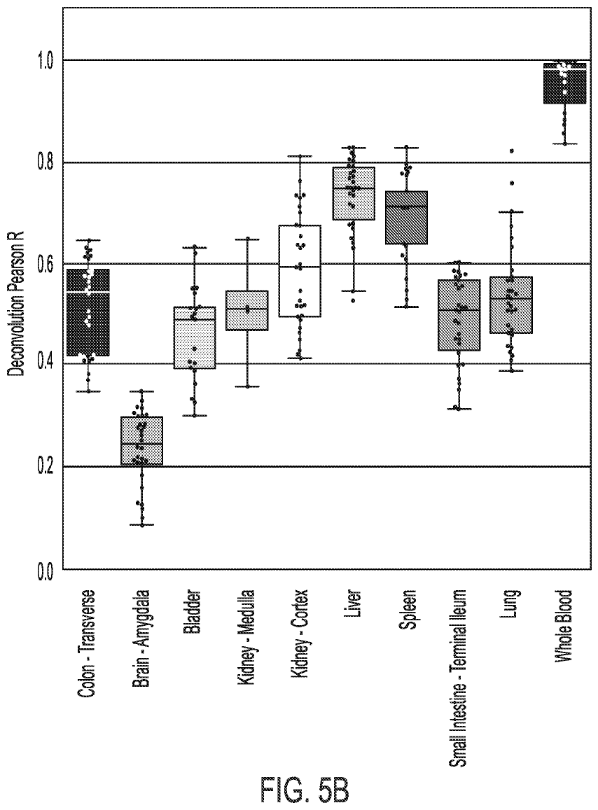

This required specifying a basis matrix with a representative gene set (rows) that could accurately and simultaneously resolve the distinct cell types (columns). To reduce multicollinearity, transcriptionally-similar cell types were grouped. The basis matrix appropriately described cell types as most similar to others from the same organ compartment, where cell types originating from the same compartment correspond to the highest off-diagonal similarity (FIG. 1c). The defined basis matrix accurately deconvolved cell-type specific RNA fractional contributions from several GTEx bulk tissue samples (FIG. 5).

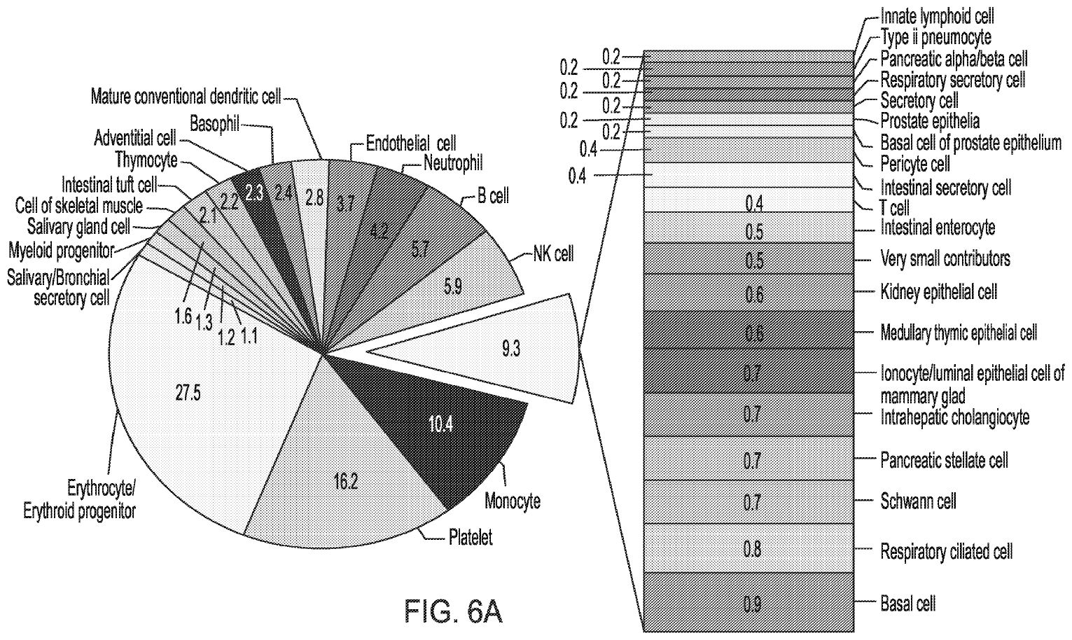

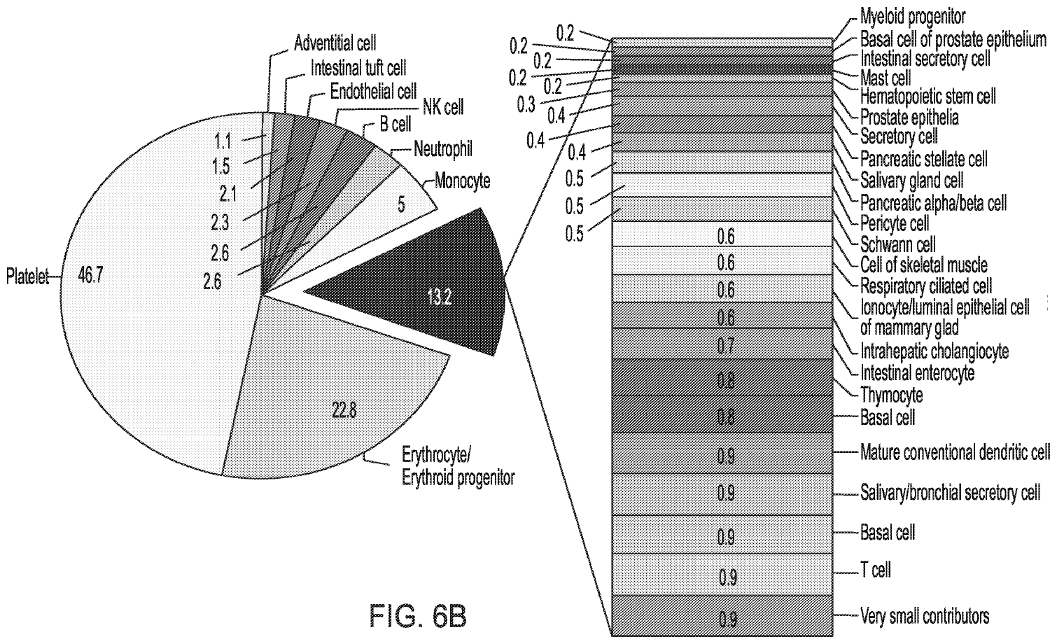

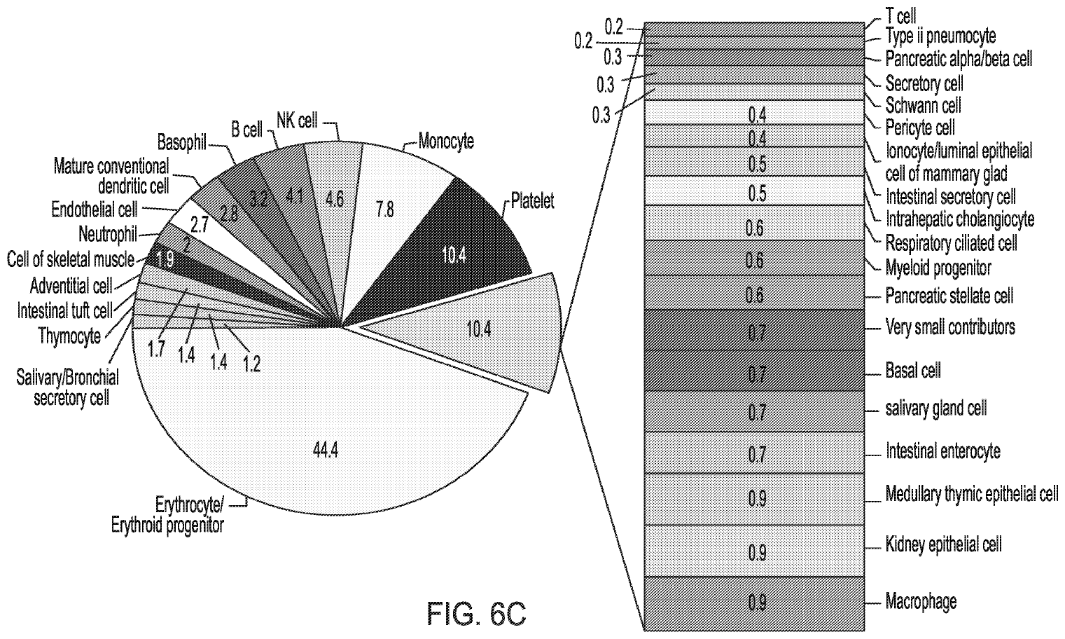

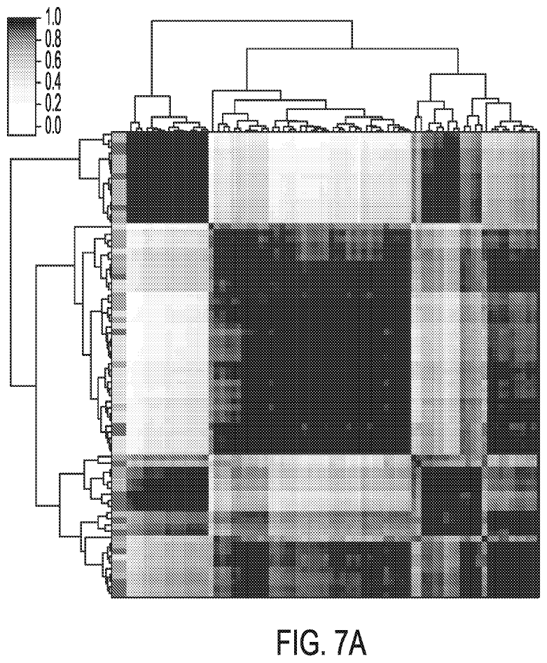

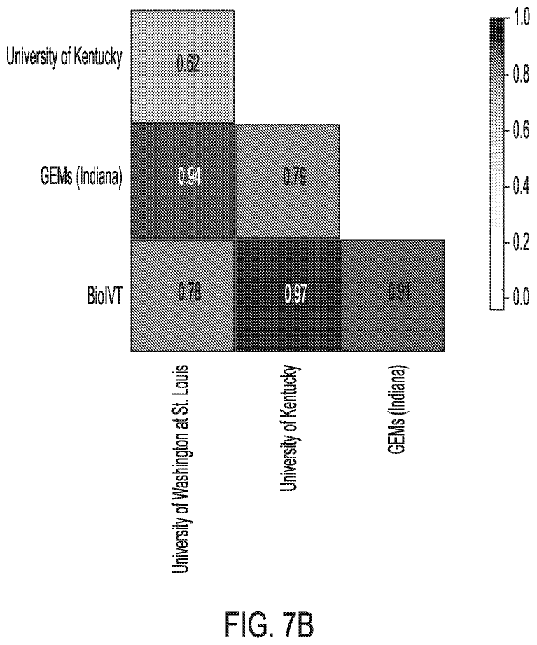

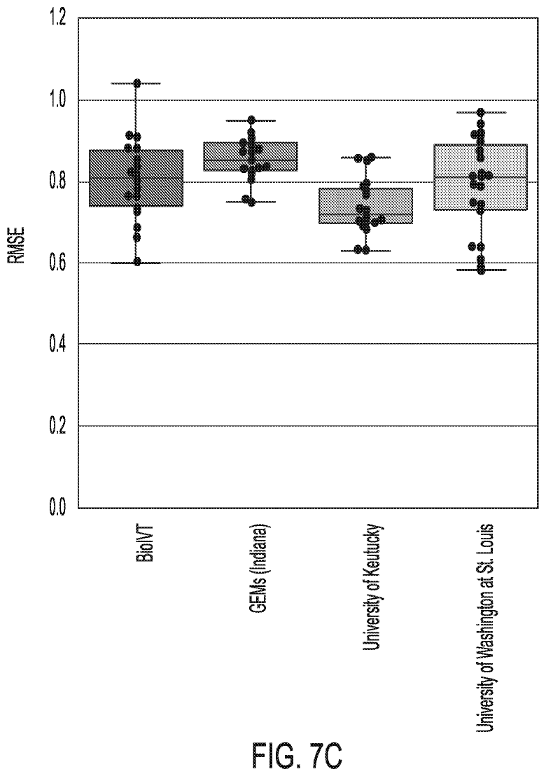

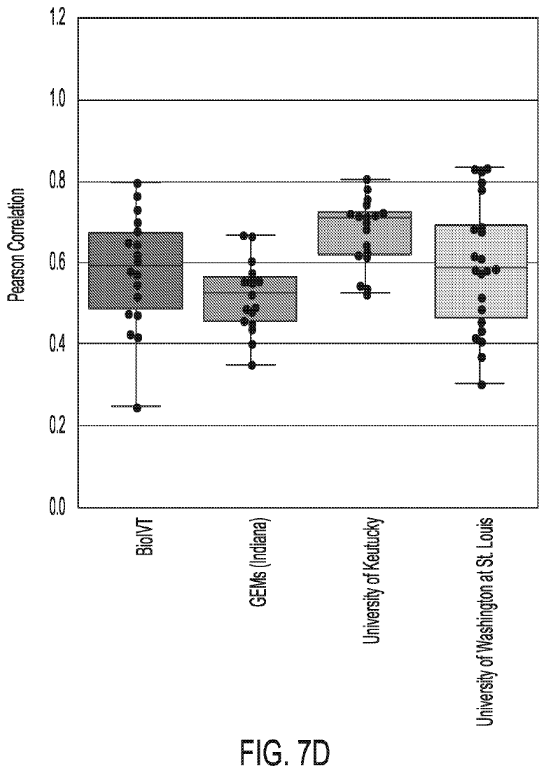

This matrix was used to deconvolve the cell types of origin in the the plasma cf-transcriptome (FIG. 1d, FIG. 6 and FIG. 7). Platelets, erythrocyte/erythroid progenitors and leukocytes comprised the majority of observed signal, whose respective proportions were generally consistent with recent estimates from serum cfRNA¹ and plasma cfDNA⁵⁴. Within this set of cell types, the observation of platelets as a majority cell type, rather than megakaryocytes¹, reflects annotation differences in reference data. Distinct transcriptional contributions from solid tissue-specific cell types from the intestine, liver, lungs, pancreas, heart, and kidney (FIG. 1d FIG. 6) were observed. Altogether, the observation of contributions from many non-hematopoietic cell types underscores the ability to simultaneously non-invasively resolve contributions to cfRNA from disparate cell types across the body.

A large signal from hematopoietic cell types, as well as smaller, distinct transcriptional contributions from tissue-specific cell types from the large and small intestine, lungs, and pancreas was also observed (FIG. 1d). The highest cell type contributors were monocytes (18.6±2.3%), platelets (13.6±3.5%), erythrocytes and erythroid progenitors (15.8±9.1%), and lymphocytes (15.7±2.7%). There was good pairwise similarity amongst all biological replicates (r≥0.66). The predominant cell types and their respective proportions observed are generally consistent with recently published estimates for serum cfRNA¹ and plasma cfDNA¹⁴. Small fractional contributions from endothelial cells, pancreatic cells, intestinal enterocytes, kidney epithelial cells, ciliated cells, brush cells, pancreatic acinar cells, and other pancreatic cells were also observed (FIG. 1d), underscoring the contributions of non-hematopoietic cell types to the cf-transcriptome.

Some cell types likely present in the plasma cf-transcriptome were not found in this decomposition because the source tissues were not represented in TSP. Although, ideally, reference gene profiles for all cell types would be simultaneously considered in this decomposition, a complete reference dataset spanning the entire cell type space of the human body does not yet exist. To identify cell type contributions possibly absent from this analysis, the genes measured in cfRNA missing from the basis matrix were intersected with tissue-specific genes from the Human Protein Atlas (HPA) RNA consensus dataset50 (FIG. 1e). This identified both the brain and the testis as tissues whose cell types were not found during systems-level deconvolution and additional genes specific to the blood, skeletal muscle, and lymphoid tissues that were not used by the basis matrix (FIG. 1e).

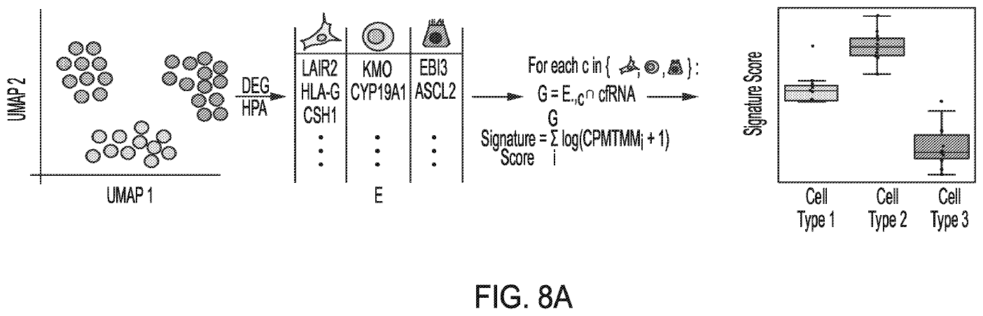

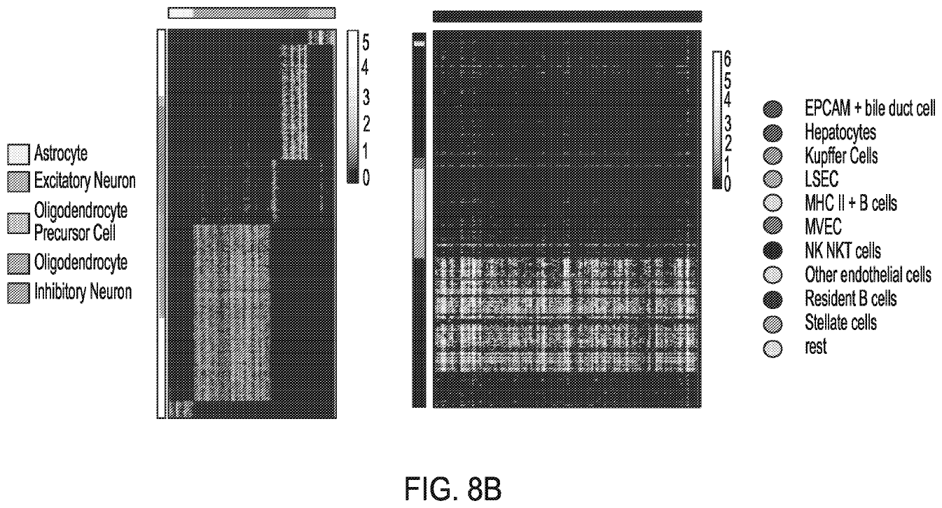

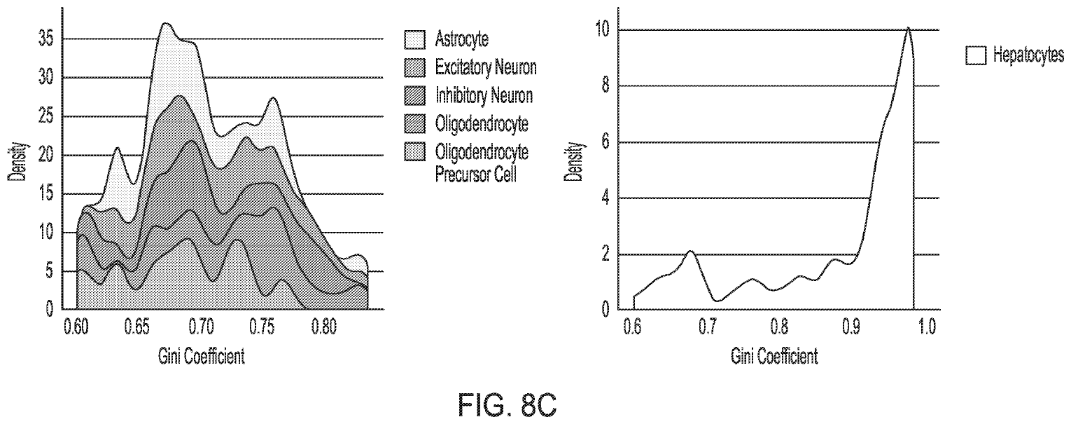

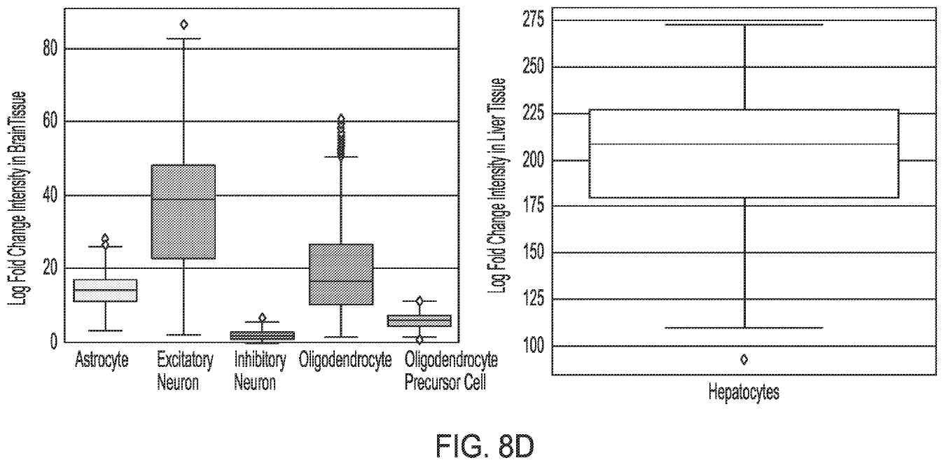

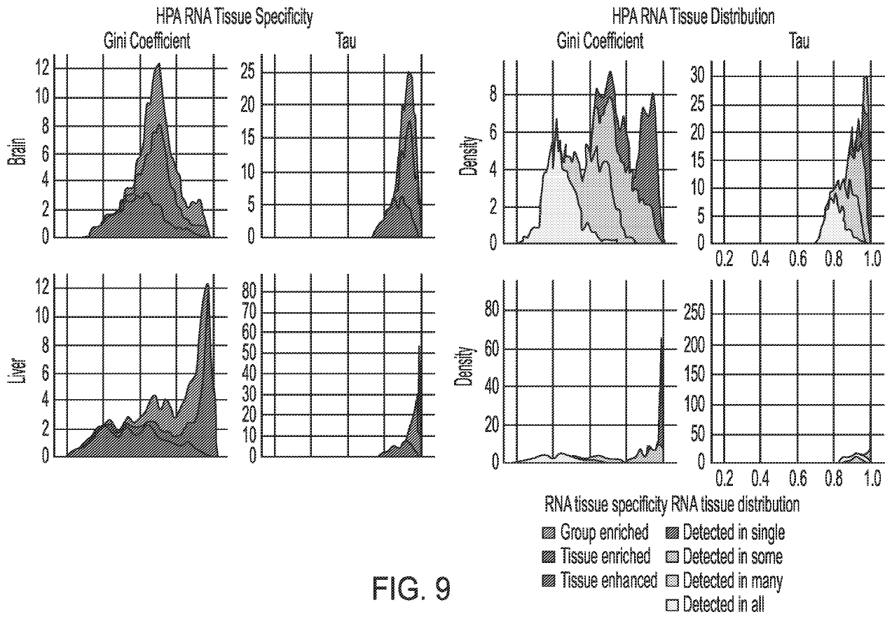

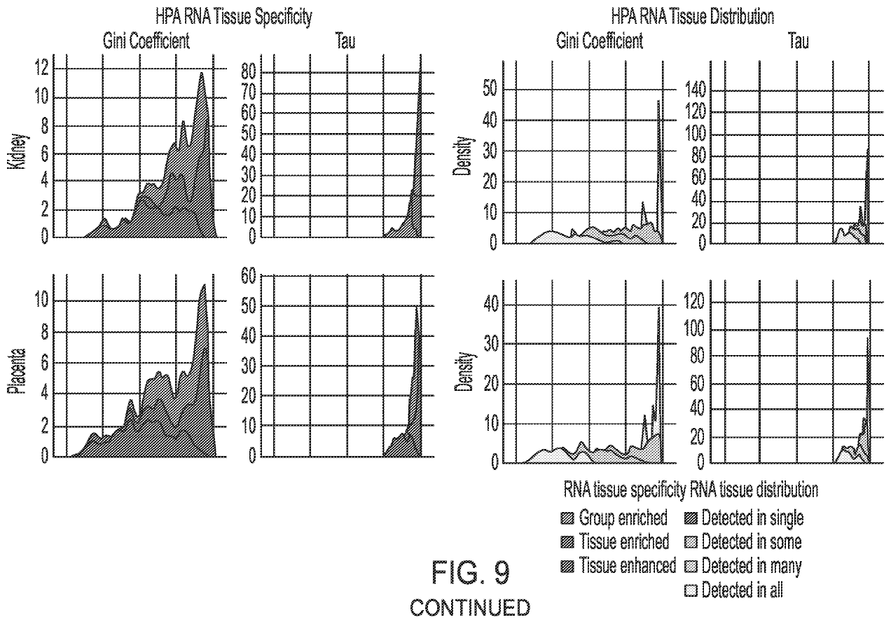

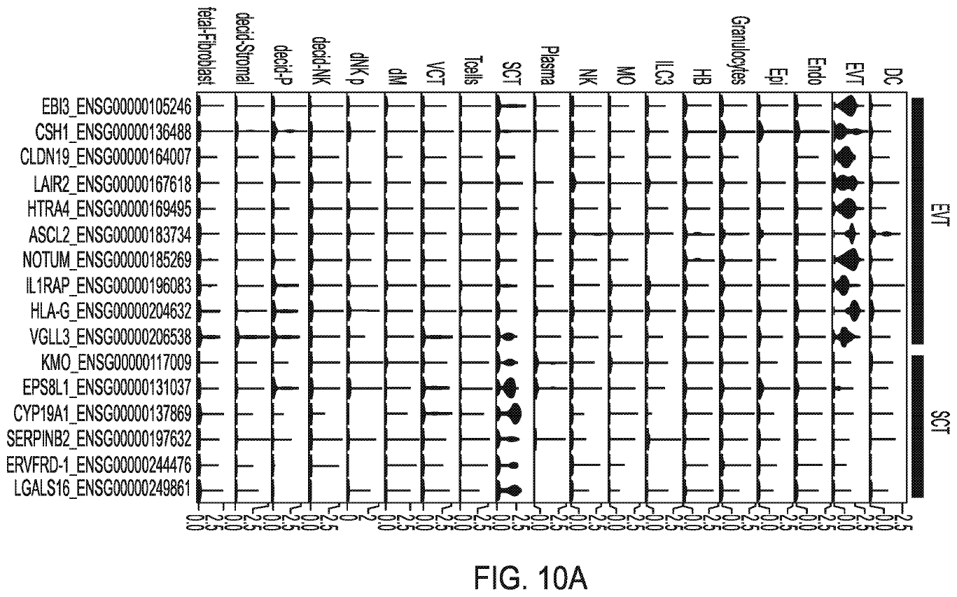

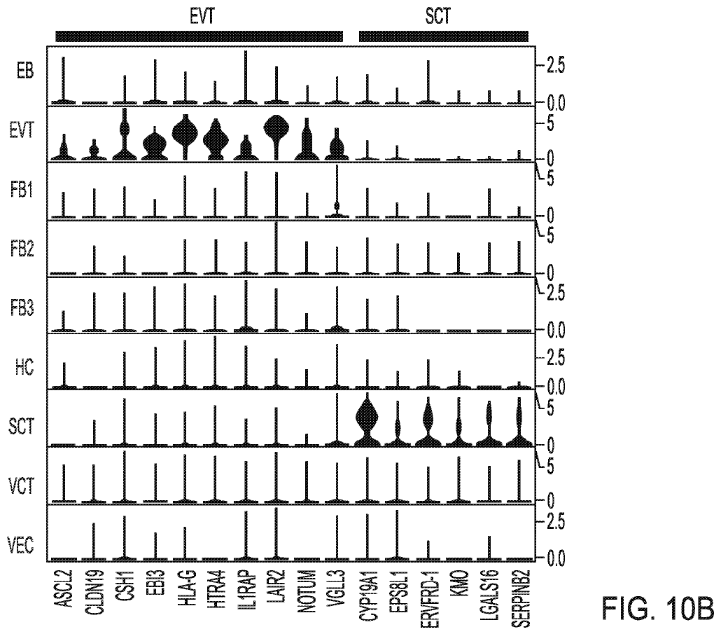

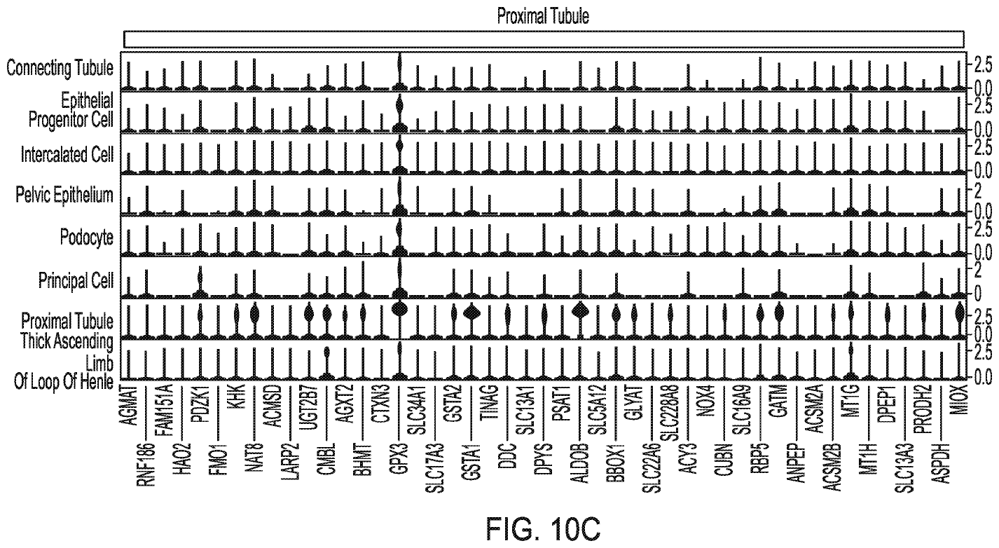

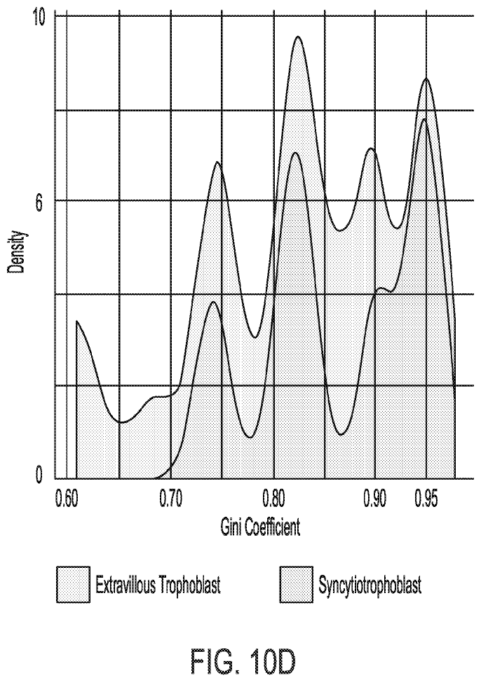

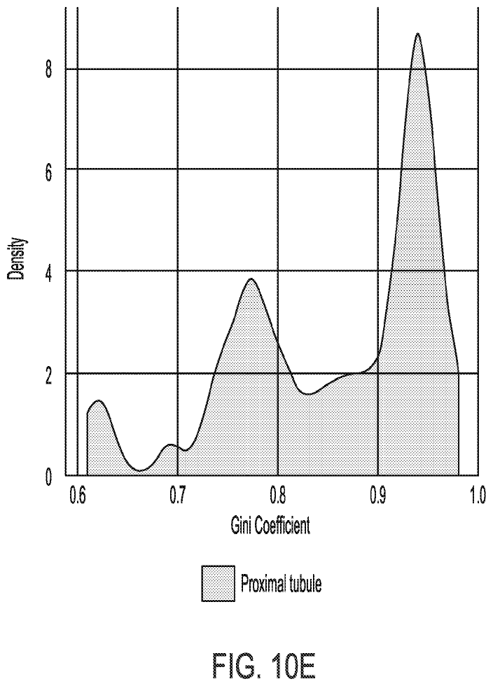

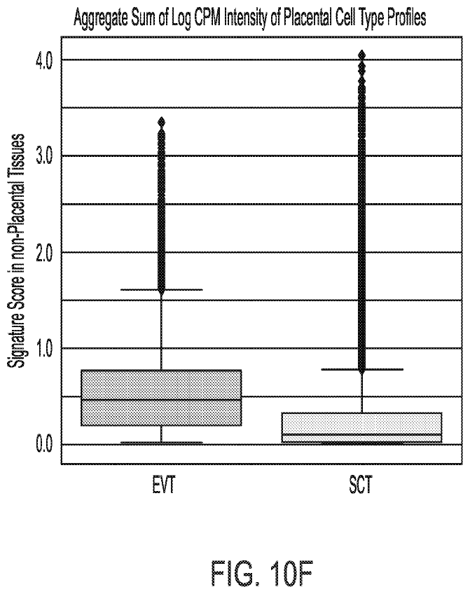

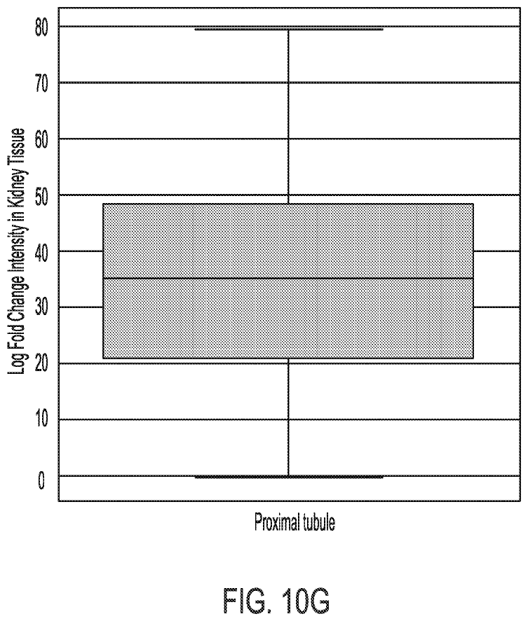

Cell type specific gene profiles were defined for these tissues in context of the whole body. To do so, individual tissue cell atlases10,55-58 were used but only considered cell types unique to a given tissue (FIG. 8 and FIG. 9). This formulation allowed us to apply bulk GTEx and HPA transcriptomic data to ensure whole body specificity using stringent expression specificity constraints. First, a given gene was required to be differentially expressed in a given cell type against all others within an individual tissue cell atlas (FIG. 8 and FIG. 10). Second, high expression inequality was required across tissues measured by the Gini coefficient⁵⁹ (FIGS. 8, 9 and 10). The specificity of a given gene profile was validated to its corresponding cell type by comparing the aggregate expression of a given cell type signature in its native tissue compared to that of the average across remaining GTEx tissues (FIG. 8 and FIG. 10). A median fold change greater than one in the signature score of a cell type gene profile in its native tissue was uniformly observed relative to the mean expression in other tissues, confirming high specificity.

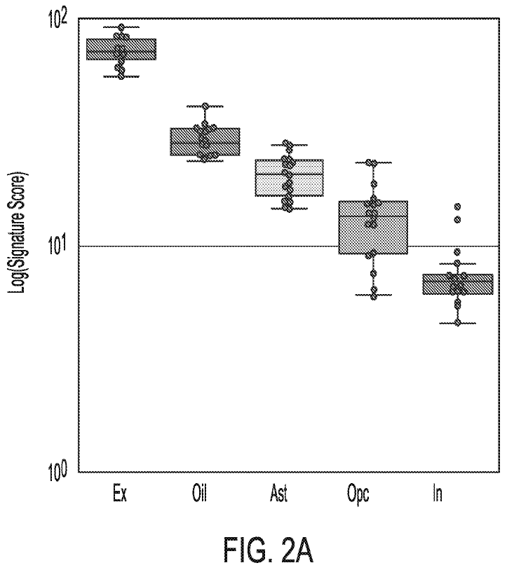

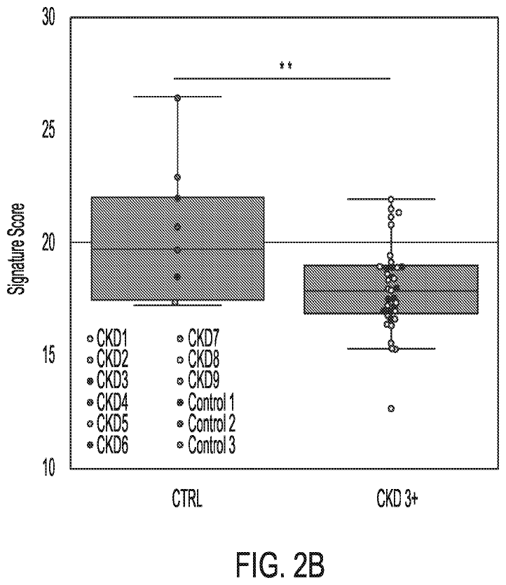

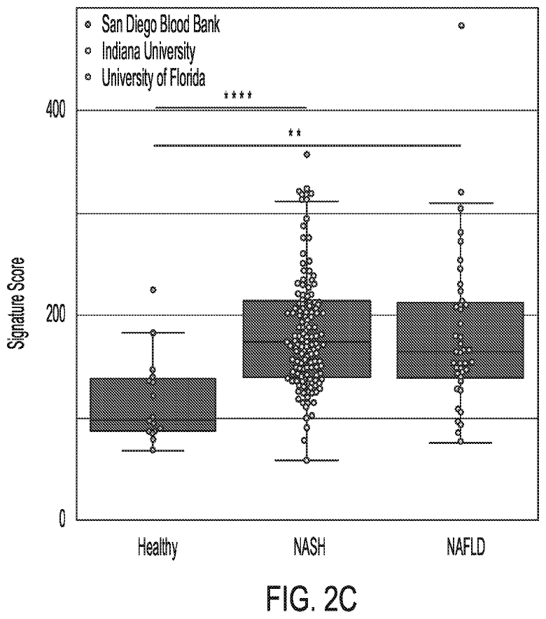

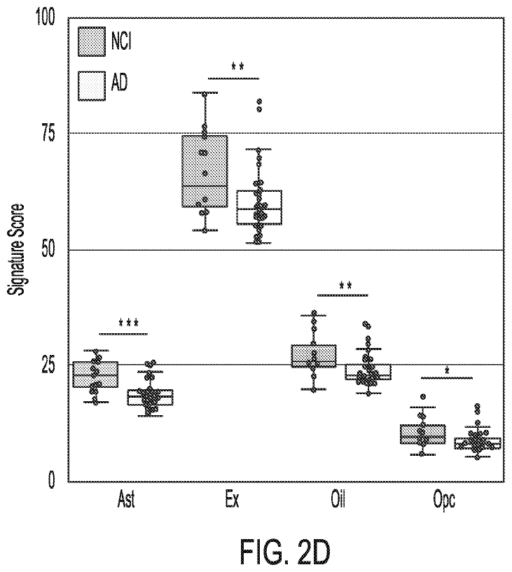

As an example of how to analyze cell type contributions from tissues that were not present in TSP, an independent brain single-cell atlas along with HPA was used to define cell type gene profiles and examined their expression in cfRNA (FIG. 2a and FIGS. 8 and 9). A signature score for each cell type in the cf-transcriptome using its specific gene profile was achieved by summing the measured level for all included genes (FIG. 2a). Specifically, a strong signature score was measured from excitatory neurons and a reduced signature score from inhibitory neurons. Strong signals fwere also observed rom astrocytes, oligodendrocytes, and oligodendrocyte precursor cells. These glial cells facilitate brain homeostasis, form myelin, and provide neuronal structure and support⁶. Evidence of RNA transport across and the permeability of the blood brain barrier (BBB)^{60,61}, and that brain regions in direct contact with the blood⁶². augment the consistent detection of brain cell type signatures in the cf-transcriptome. Similarly, published cell atlases were used for the placenta⁵⁶⁻⁵⁷, kidney⁵⁸ and liver⁵⁵ to define cell-type-specific gene profiles (FIG. 8 and FIG. 10) for signature scoring. These observations augment the resolution of previously observed tissue-specific genes reported to date in cfRNA^{1,3-5,44-46} and formed a baseline from which to measure aberrations in disease.

A strong hepatocyte signature score was also observed, which is consistent with their high turnover rate and cellular mass²², a small signal for atrial cardiomyocytes, and negligible signal from ventricular cardiomyocytes, consistent with the low level of cardiomyocyte death in healthy adults²³. These observations augment the resolution of previously observed brain-^{3,4}, liver-⁵, and heart²⁴—specific genes reported to date in cfRNA.

Plasma cfRNA Measurement Reflects Cellular Pathophysiology

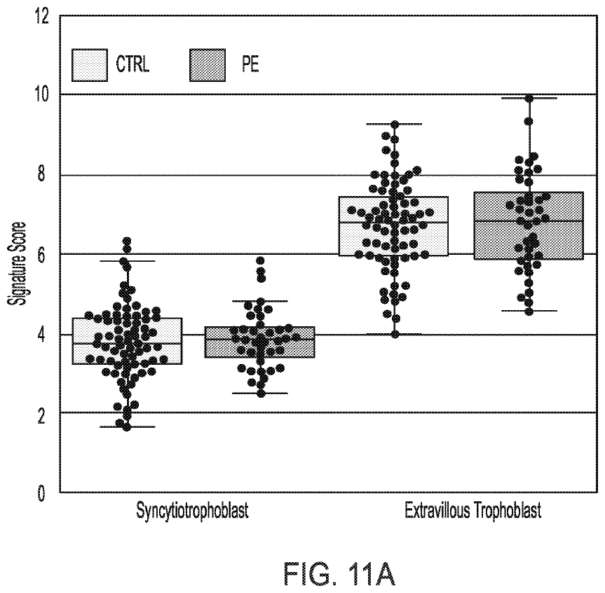

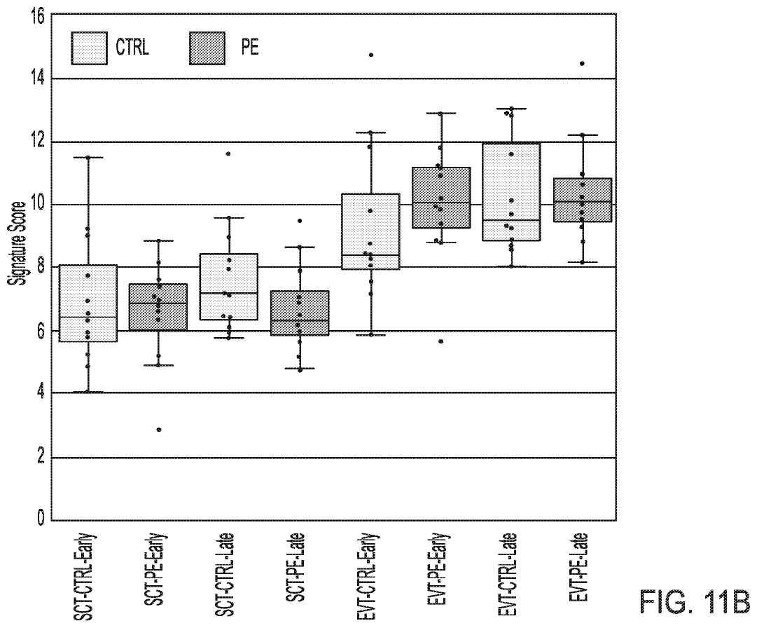

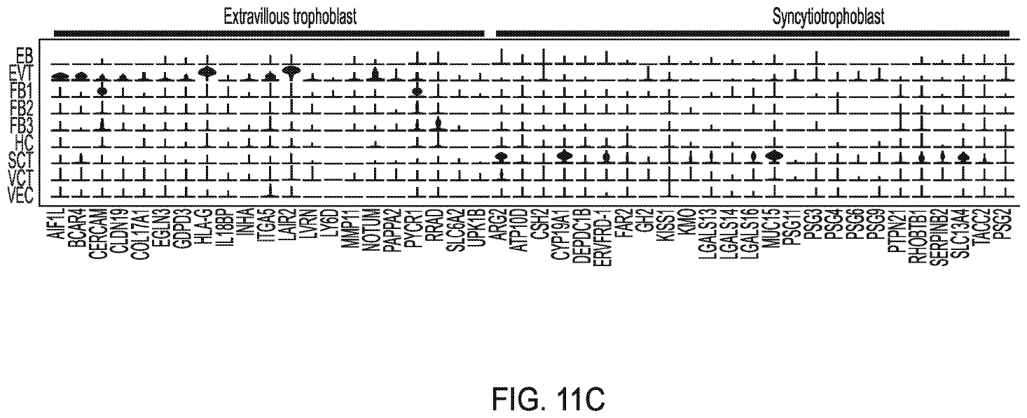

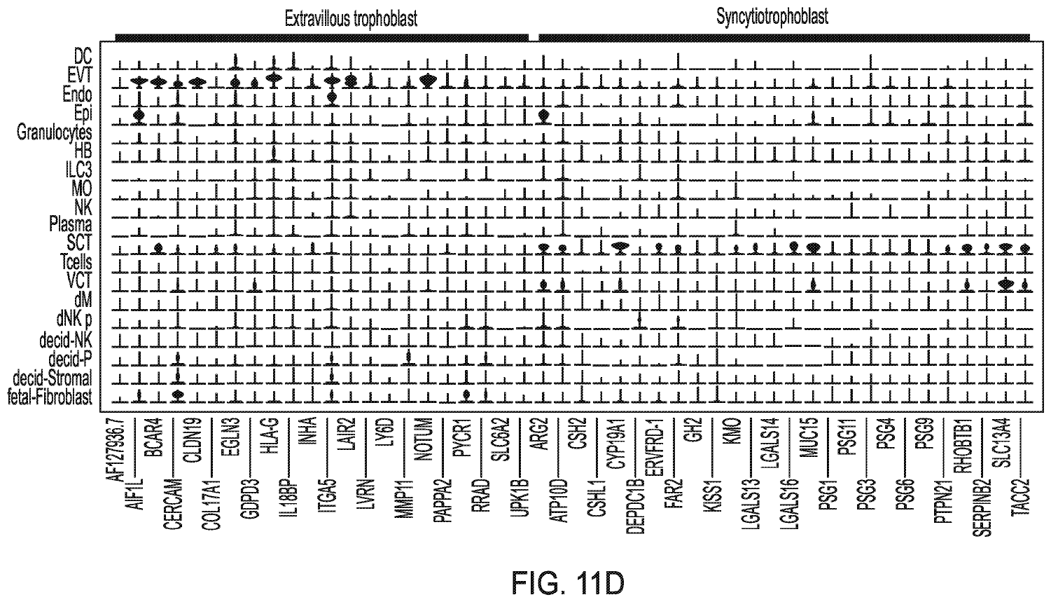

Cell-type-specific changes drive disease etiology6, and whether cfRNA reflected cellular pathophysiology was asked. As an example of why whole-body cell type characterization is relevant, a previous attempt to infer trophoblast cell types from cfRNA in preeclampsia63 used genes that are not specific or readily measurable within their asserted cell type was observed (FIG. 11).

In pregnancy, extravillous trophoblast (EVT) invasion is a stage in uteroplacental arterial remodeling57,64 Arterial remodeling occurs to ensure adequate maternal blood flow to the growing fetus57,64 and is sometimes reduced in preeclampsia64. Previously, the EVT was reported by Tsang et al to be noninvasively resolvable and elevated in early onset preeclampsia (gestational age at diagnosis<34 weeks) as compared to healthy pregnancy⁶³. However, examination of the trophoblast gene profiles used by Tsang et al. using two independent placental single-cell atlases^{56,57} revealed several genes that were not cell type specific or exhibited very low trophoblast expression (FIG. 11c, d), thereby adversely impacting signature score interpretation.

CERCAM, IL18BP, and PYCR1 are not extravillous trophoblast specific, exhibiting higher expression in fibroblast cell types in both atlases, despite Tsang's inclusion in their EVT gene profile (FIG. 11c,d). Furthermore, EVT genes in Tsang's gene profile, RRAD, SLC6A2, and UPK1B all exhibit very low EVT expression across both placental atlases. Numerous PSG genes (PSG11, PSG1/PSG2, PSG3, PSG4, PSG6, PSG9) do not exhibit high syncytiotrophoblast (SCT) expression, despite their inclusion in Tsang's SCT gene profile. GH2 either exhibits no expression or comparable non-SCT specific expression across cell types in both atlases (FIG. 11c, d).

The presence of these non-cell type specific genes in a cell type gene profile consequently impacted the interpretation of Tsang et al's signature scores. Using the criteria for deriving a given cell type gene profile (Methods), gene profiles for the same two cell types, EVT and SCT (FIG. 10) were drived, and then quantified their respective signature scores in two previously published preeclampsia cohorts⁴⁶ (FIG. 11a, b). In contrast to Tsang et al, no significant difference was observed in either trophoblast signature score in cfRNA samples collected at diagnosis for mothers with early-onset preeclampsia (p=0.703 and U=1524, 0.794 and U=1504 respectively, two-sided Mann Whitney U) (FIG. 11a) and for mothers with either early- or late-onset preeclampsia (p=0.24 and H=4.18, 0.54 and H=2.15 respectively, Kruskal Wallace) (FIG. 11b), as compared to samples from mothers with no complications at a matched gestational age.

In the present disclosure deriving cell type gene profiles for signature scoring in cfRNA, genes with high log fold change in a given cell type population and low expression in any other measured cell type were solely considered.

Taken together with validation in two independent placental cell atlases, the EVT and SCT cell type gene profiles by Tsang et al. do not enable estimation of trophoblast pathology from cfRNA in preeclampsia. The role of extravillous trophoblast invasion and the ubiquity of its cellular pathophysiology in preeclampsia thus remains an open question.

However, several other cases were found where cellular pathophysiology can be measured in cfRNA. Proximal tubules in chronic kidney disease (CKD)⁶⁵⁻⁶⁷, hepatocytes in non-alcoholic steatohepatitis (NASH)/non-alcoholic fatty liver disease (NAFLD)² and multiple brain cell types in Alzheimer's disease (AD)^{10,68} were each considered.

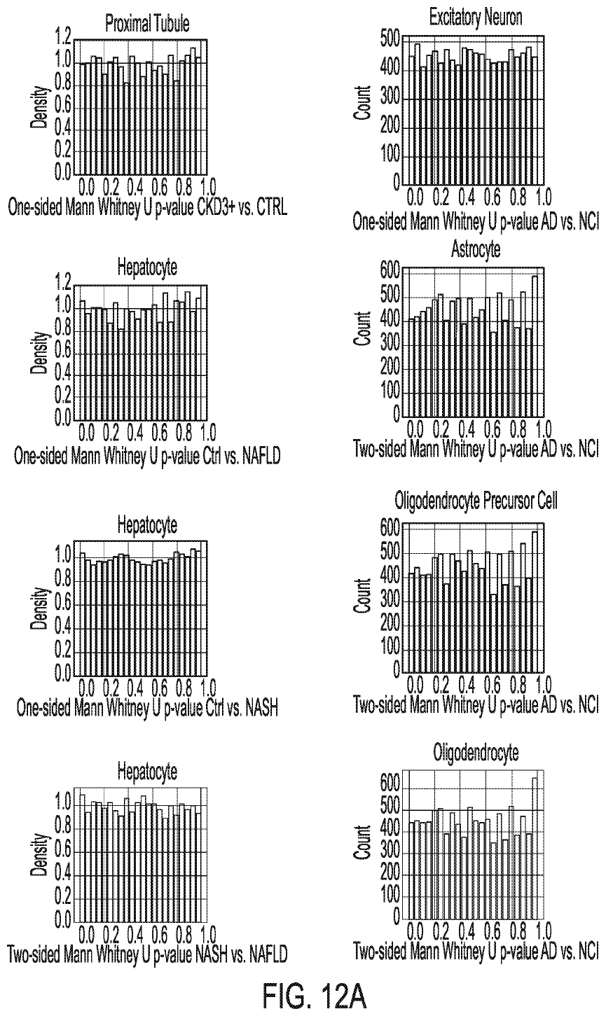

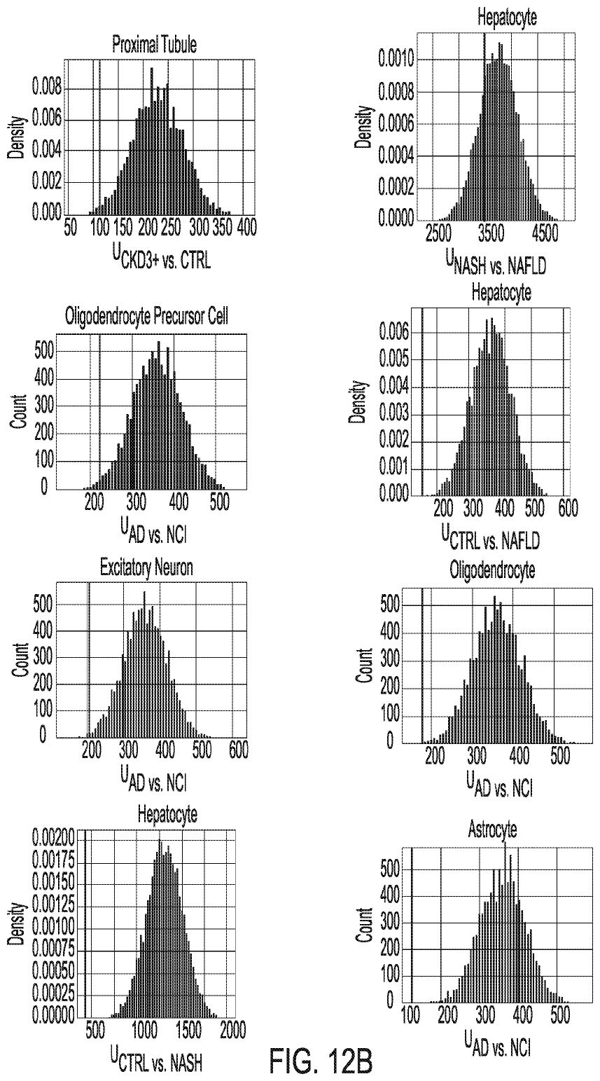

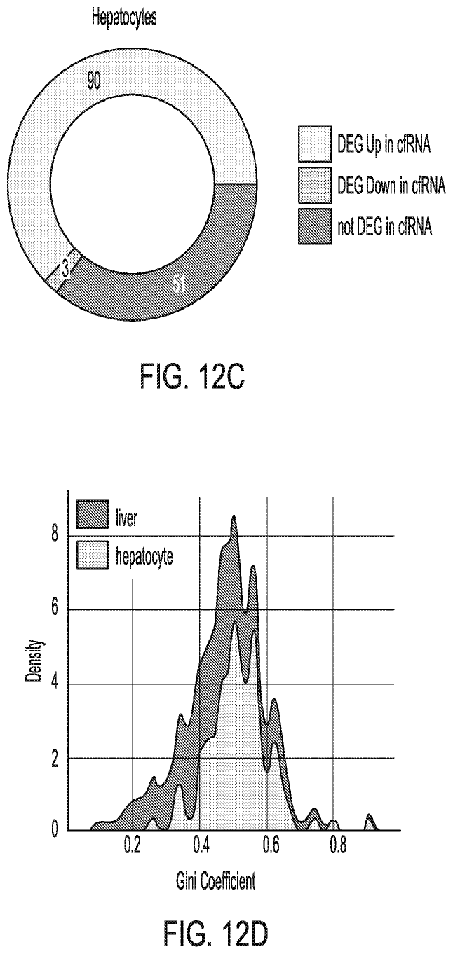

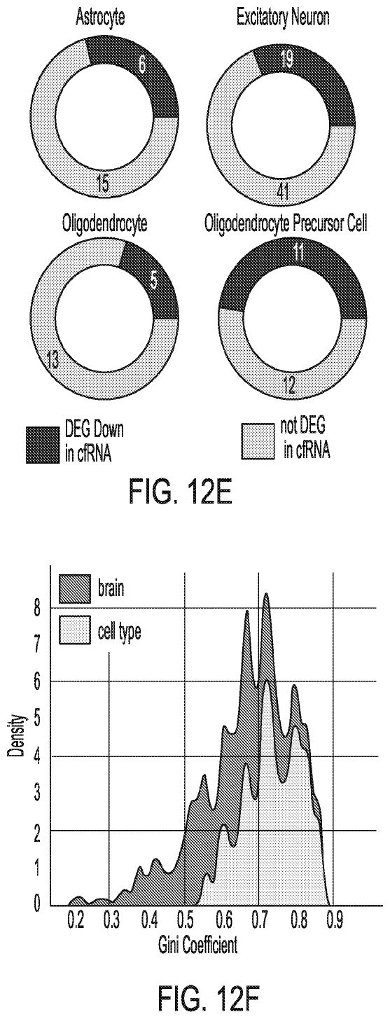

The proximal tubule is a highly metabolic, predominant kidney cell type and is a major source for injury and disease progression in CKD⁶⁵⁻⁶⁷. Tubular atrophy is a hallmark of CKD nearly independent of disease etiology⁶⁹ and is superior to clinical gold standard as a predictor of CKD progression³⁵. Using data from Ibarra et al., a striking decrease was discovered in the proximal tubule cell signature score of patients with CKD (ages 67-91 years, CKD stage 3-5 or peritoneal dialysis) compared to healthy controls (FIG. 2b and FIG. 12a, b). These results demonstrate non-invasive resolution of proximal tubule deterioration observed in CKD histology³⁵ and are consistent with findings from invasive biopsy.

Hepatocyte steatosis is a histologic hallmark of NASH and NAFLD phenotypes, whereby the accumulation of cellular stressors results in hepatocyte death²⁵. Several genes differentially expressed in NAFLD serum cfRNA⁵ were specific to the hepatocyte cell type profile derived above (P<10⁻¹⁰, hypergeometric test). Notable hepatocyte-specific differentially expressed genes (DEGs) include genes encoding cytochrome P450 enzymes (including CYP1A2, CYP2E1 and CYP3A4), lipid secretion (MTTP) and hepatokines (AHSG and LECT2)⁷⁰. Striking differences were further observed in the hepatocyte signature score between healthy and both NAFLD and NASH cohorts and no difference between the NASH and NAFLD cohorts (FIG. 2c and FIG. 12).

AD pathogenesis results in neuronal death and synaptic loss⁶⁸. Brain single-cell data¹⁰ was used to define brain cell type gene profiles in both the AD and the normal brain. Several DEGs found in cfRNA analysis of AD plasma are brain cell type specific (P<10⁻⁵, hypergeometric test). Astrocyte-specific genes include those that encode filament protein (GFAP⁷¹) and ion channels (GRIN2C¹⁰). Excitatory neuron-specific genes encode solute carrier proteins (SLC17A7¹⁰) and SLC8A2⁷²), cadherin proteins (CDH8⁷³ and CDH22⁷⁴) and a glutamate receptor (GRM1^{68,75}). Oligodendrocyte-specific genes encode proteins for myelin sheath stabilization (MOBP⁸) and a synaptic/axonal membrane protein (CNTN2⁶⁸). Oligodendrocyte-precursor-cell-specific genes encode transcription factors (OLIG2⁷⁶ and MYT1⁷⁷), neural growth and differentiation factor (CSPG⁷⁸) and a protein putatively involved in brain extracellular matrix formation (BCAN⁷⁹).

Neuronal death in plasma cfRNA between AD and healthy non-cognitive controls (NCIs) was then inferred and also observed differences in oligodendrocyte, oligodendrocyte progenitor and astrocyte signature scores (FIG. 2d and FIG. 12). The oligodendrocyte and oligodendrocyte progenitor cells signature score directionality agrees with reports of their death and inhibited proliferation in AD, respectively⁸⁰. The observed astrocyte signature score directionality is consistent with the cell type specificity of a subset of reported downregulated DEGs⁴ and reflects that astrocyte-specific changes, which are known in AD pathology⁸⁰, are non-invasively measurable.

The cell type gene profiles provided herein include those responsible for drug metabolism (for example, liver and renal cell types) as well as cell types that are drug targets, such as neurons or oligodendrocytes.

Drugs are hepatically and/or renally metabolized and can damage cell types in these organs, hepatotoxic and nephrotoxic drugs respectively. Logical extensions of these gene profiles will reveal physiological disruptions to these organs include monitoring drug toxicity and response. Comparison to a control value would reveal a difference in signature scores of these cell types upon drug administration and would reflect cell type death.

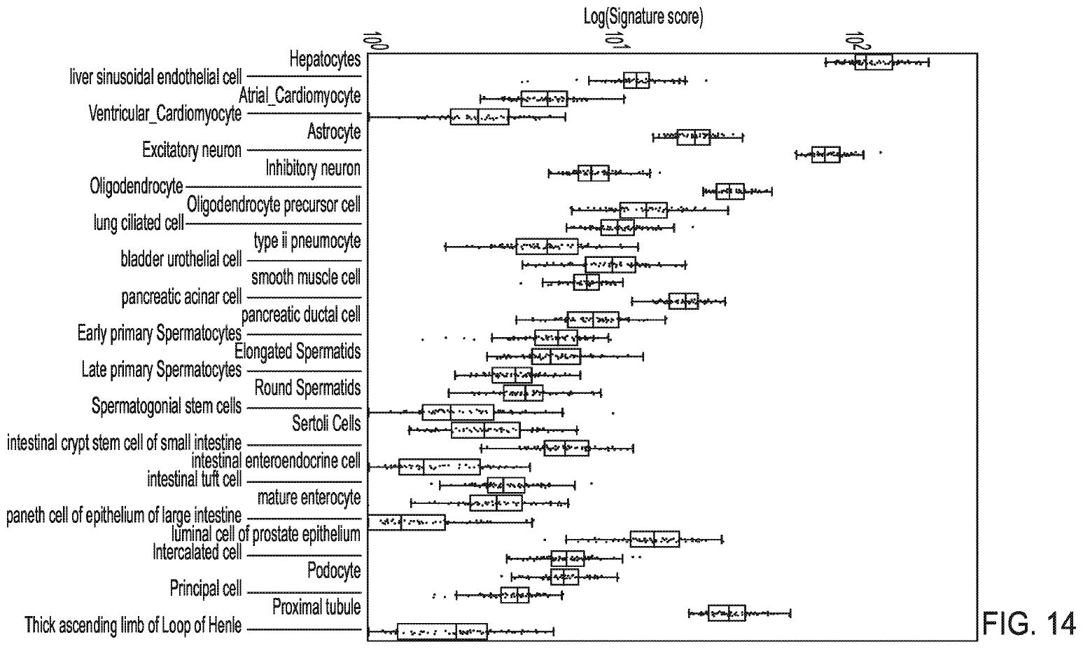

A broad spectrum of cell type specific signal in the healthy cf-transcriptome was observed following signature score estimation for each cell type gene profile originating from the liver, heart, normal brain, lung, bladder, pancreas, testis, intestine, prostate, and kidney (FIG. 14). The ability to readily measure this broad landscape of cell type specific signal in the healthy plasma cf-transcriptome establishes a baseline from which changes in a given disease in any of these tissues can be noninvasively measured at cellular resolution. Given the number of diseases (CKD, AD, NASH/NAFLD as well as preeclampsia) (Vorperian, S., et al., Nature Biotech., 2022, doi.org/10.1038/s41587-021-01188-9; Moufarrej, M., et al., Nature, 2022, 602:689-694) that have been measured for cell-type specific differences in disease using derived gene profiles, the present methods are applicable for the noninvasive measurement of a given cell type in disease.

Taken together, present disclosure demonstrates consistent, non-invasive detection of cell-type-specific changes in human health and disease using cfRNA. The present disclosure upholds and further augments the scope of previous work identifying immune cell types¹ and hematopoietic tissues^{1,3} as primary contributors to the cell-free transcriptome cell type landscape. The present disclosure methods are, in some embodiments, complementary to previous work using cell-free nucleosomes⁵⁴, which depends on a more limited set of reference chromatin immunoprecipitation sequencing data, which are largely at the tissue level⁸¹. Readily measurable cell types include those specific to the brain, lung, intestine, liver, and kidney, whose pathophysiology affords broad prognostic and clinical importance.

Consistent detection of cell types responsible for drug metabolism (for example, liver and renal cell types) as well as cell types that are drug targets, such as neurons or oligodendrocytes for Alzheimer's-protective drugs, could provide strong clinical trial endpoint data when evaluating drug toxicity and efficacy. The ability to non-invasively resolve cell type signatures in plasma cfRNA will both enhance existing clinical knowledge and enable increased resolution in monitoring disease progression and drug response.

### Discussion

Recent efforts to noninvasively identify the origins of circulating nucleic acids underscore the reference data limitations and the importance of reporting cell-type of origin. Recent efforts to identify tissue-of-origin in whole blood using bulk tissue data are confounded by cellular heterogeneity driving variance in reference tissue data⁸² and lacks the resolution afforded by cell type-of-origin. The present approach to determine the signature score for a cell type of interest in cfRNA leverages the myriads of single cell transcriptomic atlases in health and disease.

The present disclosure, in some embodiments, underscores the importance of reference data annotation at both bulk tissue and single cell level; differences in either impact the ability to meaningfully integrate reference data to analyze cfRNA. Cell type annotation differences across distinct tissue cell atlases may conflate the assignment of a gene as cell-type specific when considering a single dataset. Specifically, several genes reported as specific to a single trophoblast cell type⁶³ were not validated in two independent placental cell atlases^{56,57}. Annotation discrepancies between atlases impacts the assignment of genes as cell type specific in context of the whole body, and consequently impact the interpretation of a cell type signature score in cfRNA.

In some embodiments, the present disclosure shows that atlases can be applied to measure disparate cell types that are disease-implicated in the blood, relevant to a myriad of questions impacting human health. Unlike model organisms which lack full translatability to human health, cf-transcriptomic measurement provides direct, immediate insights into patient health. Readily measurable cell types in cfRNA, including those specific to the brain, lung, intestine, liver, and kidney, have vast prognostic and clinical importance given the multitude of diseases in these tissues. Single cell RNA-seq reveals numerous cell type specific changes in pathologies within these tissues for investigation with cfRNA ranging from cancer to Crohn's disease, drug or vaccine response, and aging.

### Materials & Methods

### Data Processing

### Data Acquisition

cfRNA: For samples from Ibarra et al. (PRJNA517339), Toden et al. (PRJNA574438) and Chalasani et al. (PRJNA701722), raw sequencing data were obtained from the Sequence Read Archive with the respective accession numbers. For samples from Munchel et al., processed counts tables were directly downloaded.

For all individual tissue single-cell atlases, Seurat objects or AnnData objects were downloaded or directly received from the authors. Data from Mathys et al. were downloaded with permission from Synapse. The liver Seurat object was requested from Aizarani et al. For the placenta cell atlases, a Seurat object was requested from Suryawanshi et al., and AnnData was requested from Vento-Tormo et al. Kidney AnnData were downloaded (www.kidneycellatlas.org, Mature Full dataset).

HPA Version 19 Transcriptomic Data. Genotype-Tissue Expression (GTEx) Version 8 Raw Counts and Tabula Sapiens Version 1.0 were Downloaded Directly.

### Bioinformatic Processing

All analyses were performed using Python (version 3.6.0) and R (version 3.6.1) For each sample for which raw sequencing data were downloaded, reads were trimmed using trimmomatic (version 0.36) and then mapped them to the human reference genome (hg38) with STAR (version 2.7.3a). Duplicate reads were then marked and removed by the MarkDuplicates tool in GATK (version 4.1.1). Finally, mapped reads were quantified using htseq-count (version 0.11.1), and read statistics were estimated using FastQC (version 0.11.8).

The bioinformatic pipeline was managed using snakemake (version 5.8.1). Read and tool performance statistics were aggregated using MultiQC (version 1.7).

### Sample Quality Filtering

For every sample for which raw sequencing data were available, three quality parameters were estimated as previously described^{83,84}: RNA degradation, ribosomal read fraction and DNA contamination.

RNA degradation was estimated by calculating a 3′ bias ratio. Specifically, the number of reads per exon were first counted and then annotated each exon with its corresponding gene ID and exon number using htseq-count. Using these annotations, the frequency of genes for which all reads mapped exclusively to the 3′-most exon were measured as compared to the total number of genes detected. RNA degradation was approximated for a given sample as the fraction of genes where all reads mapped to the 3′-most exon.

To estimate ribosomal read fraction, the number of reads that mapped to the ribosome (region GL00220.1:105,424-118,780, hg38) were compared relative to the total number of reads (SAMtools view).

To estimate DNA contamination, an intron-to-exon ratio was used and quantified the number of reads that mapped to intronic as compared to exonic regions of the genome.

The following thresholds were applied as previously reported⁸³:
Ribosomal: >0.2
3′ Bias Fraction: >0.4
DNA Contamination: >3

Any given sample was considered as low quality if its value for any metric was greater than any of these thresholds, and the sample was excluded from subsequent analysis.

### Data Normalization

All gene counts were adjusted to counts per million (CPM) reads and per milliliter of plasma used. For a given sample, i denotes gene index, and j denotes sample index:

$${η}_{i j} = \frac{{Gene}_{ij}}{( {LibrarySize}_{j} ) * ( mL {Plasma}_{j} )} \qquad ( 1 )$$

$$where$$

$$Library {Size}_{j} = {\sum }_{i} {G}_{ij}$$

For individuals who had samples with multiple technical replicates, these plasma volume CPM counts were averaged before nu support vector regression (nu-SVR) deconvolution.

For all analyses except nu-SVR (all work except FIG. 1d,e), trimmed mean of M values (TMM) normalization was next applied as previously described⁸⁵ using edgeR (version 3.28.1):

$$\frac{{n}_{ij}}{T M {M}_{j}} \qquad ( 2 )$$

(2)

CPM-TMM normalized gene counts across technical replicates for a given biological replicate were averaged for the count tables used in all analyses performed.

Sequencing batches and plasma volumes were obtained from the authors in Toden et al. and Chalasani et al. for per-sample normalization. For samples from Ibarra et al., plasma volume was assumed to be constant at 1 ml, sequencing batches were confirmed with the authors (personal communication). All samples from Munchel et al. were used to compute TMM scaling factors, and 4.5 ml of plasma⁴⁶ was used to normalize all samples within a given dataset (both PEARL-PEC and iPEC).

### Zero-Centered Batch Normalization

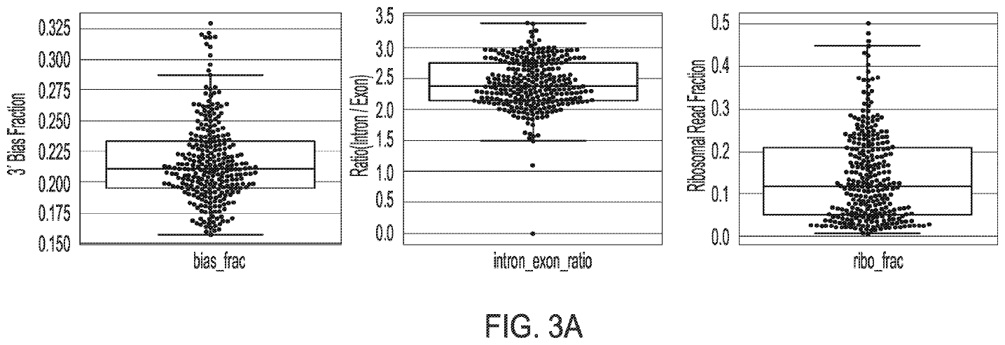

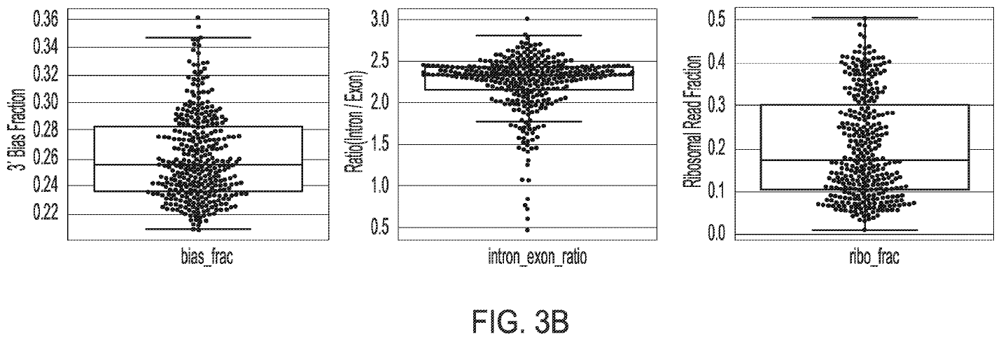

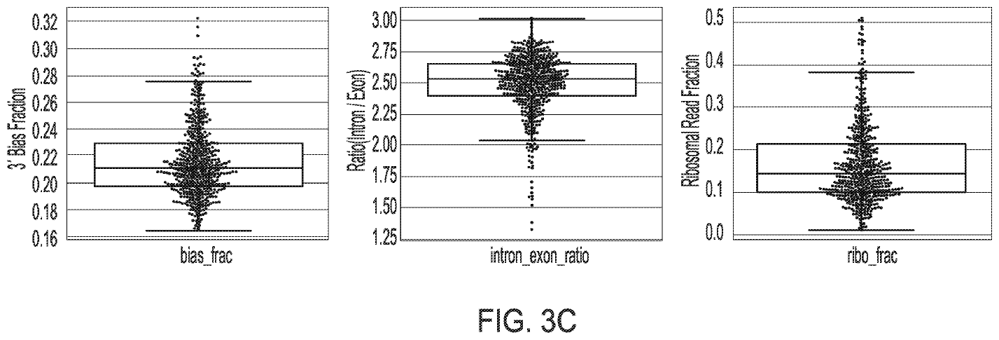

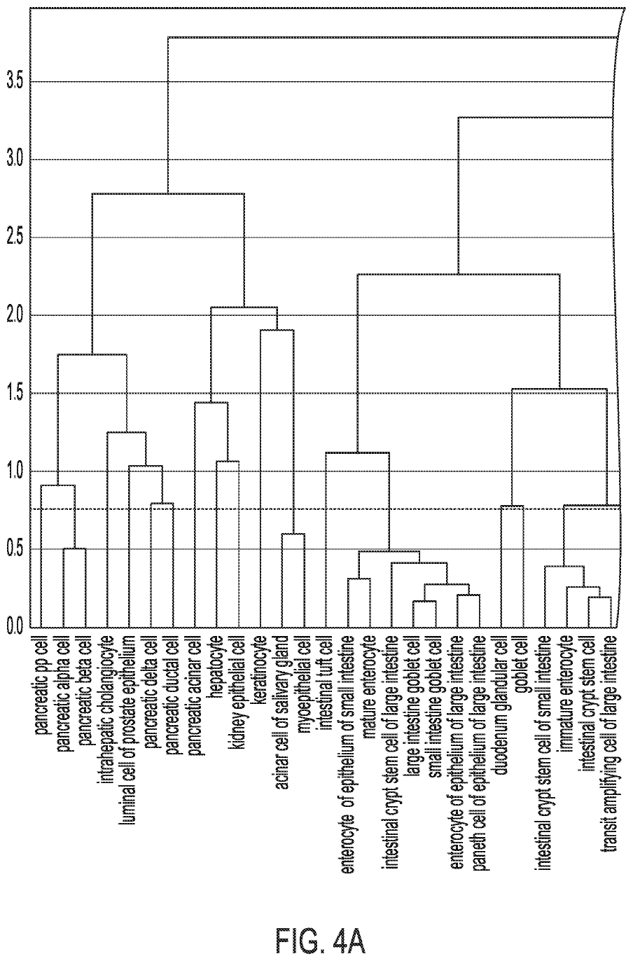

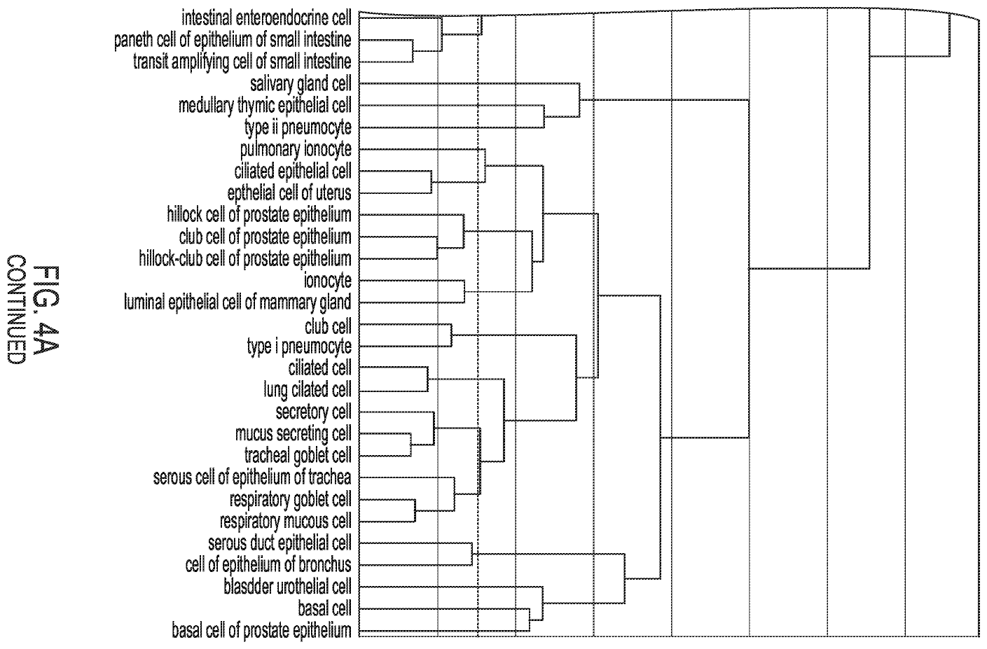

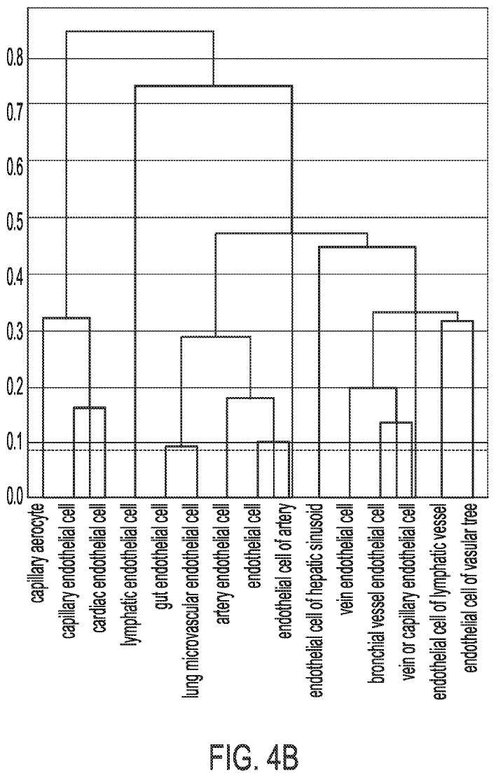

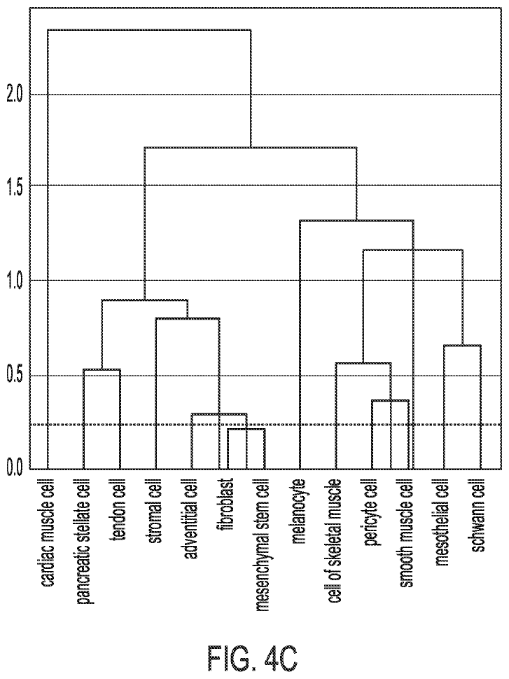

To account for center-specific effects that could impact meaningful comparison of data across centers in FIG. 3, the mean normalized value was subtracted across all samples measurements for given gene within a given batch from the measured normalized value for a given sample⁴:

G̅ᵢ̅ⱼ̅=Gᵢⱼ−μᵢₖ (3)

Where the gene index is i, the sample is j, and k is the batch. The mean expression of the i^{th} gene in the k^{th} batch is denoted by μᵢₖ.

### Cell Type Marker Identification Using PanglaoDB

The PanglaoDB cell type marker database was downloaded on 27 Mar. 2020. Markers were filtered for human (‘Hs’) only and for PanglaoDB's defined specificity (how often marker was not expressed in a given cell type) and sensitivity (how frequently marker is expressed in cells of this type). Gene synonyms from Panglao were determined using MyGene version 3.1.0 to ensure full gene space.

This gene space was the intersected with a cohort of healthy cfRNA samples (n=75, NCI individuals from Toden et al.). A given cell type marker was counted in a given healthy cfRNA sample if its gene expression was greater than zero in log+1 transformed CPM-TMM gene count space.

Cell types with markers filtered by sensitivity=0.9 and specificity=0.2 and samples with >5 cell type markers on average are shown in FIG. 1b.

### Basis Matrix Formation

Scanpy⁸⁶ (version 1.6.0) was used. Only cells from droplet sequencing (‘10x’) were used in analysis given that a more comprehensive set of unique cell types across the tissues in Tabula Sapiens was available⁴⁸. Disassociation genes as reported⁴⁸ were eliminated from the gene space before subsequent analysis.

Given the non-specificity of the following annotations (for example, other cell type annotations at finer resolution existed), cells with these annotations were excluded from subsequent analysis:
‘epithelial cell’
‘ocular surface cell’
‘radial glial cell’
‘lacrimal gland functional unit cell’
‘connective tissue cell’
‘corneal keratocyte’
‘ciliary body’
‘bronchial smooth muscle cell’
‘fast muscle cell’
‘muscle cell’
‘myometrial cell’
‘skeletal muscle satellite stem cell’
‘slow muscle cell’
‘tongue muscle cell’
‘vascular associated smooth muscle cell’
‘alveolar fibroblast’
‘fibroblast of breast’
‘fibroblast of cardiac tissue’
‘myofibroblast cell’

All additional cells belonging to the ‘Eye’ tissue were excluded from subsequent analysis given discrepancies in compartment and cell type annotations and the unlikelihood of detecting eye-specific cell types. The resulting cell type space still possessed several transcriptionally similar cell types (for example, various intestinal enterocytes, T cells or dendritic cells), which, left unaddressed, would reduce the linear independence of the basis matrix column space and, hence, would affect nu-SVR deconvolution.

Cells were, therefore, assigned broader annotations on a per-compartment basis as follows:

Epithelial, Stromal, Endothelial: Using counts from the ‘decontXcounts’ layer of the adata object, cells were CPM normalized (sc.pp.normalize_total(target_sum=1×10⁶) and log-transformed (sc.pp.log1p). Hierarchical clustering with complete linkage (sc.tl.dendrogram) was performed per compartment on the feature space comprising the first 50 principal components (sc.pp.pca). Epithelial and stromal compartment dendrograms were then cut (scipy.cluster.hierarchy.cut_tree) at 20% and 10% of the height of the highest node, respectively, such that cell types with high transcriptional similarity were grouped together, but overall granularity of the cell type labels was preserved. This work is available in the script ‘treecutter.ipynb’ on GitHub; the scipy version used is 1.5.1.

The endothelial compartment dendrogram revealed high transcriptional similarity across all cell types (maximum node height=0.851) compared to epithelial (maximum node height=3.78) and stromal (maximum node height=2.34) compartments (Extended Data FIG. 2). To this end, only the ‘endothelial cell’ annotation was used for the ‘endothelial’ compartment.

Immune: Given the high transcriptional similarity and the varying degree of annotation granularity across tissues and cell types, cell types were grouped on the basis of annotation. The following immune annotations were kept:
‘b cell’
‘basophil’
‘erythrocyte’
‘erythroid progenitor’
‘hematopoietic stem cell’
‘innate lymphoid cell’
‘macrophage’
‘mast cell’
‘mature conventional dendritic cell’
‘microglial cell’
‘monocyte’
‘myeloid progenitor’
‘neutrophil’
‘nk cell’
‘plasma cell’
‘plasmablast’
‘platelet’
‘t cell’
‘thymocyte’

All other immune compartment cell type annotations were excluded for being too broad when more detailed annotations existed (that is, ‘granulocyte’, ‘leucocyte’ and ‘immune cell’) or present in only one tissue (that is, ‘erythroid lineage cell’; eye, ‘myeloid cell’; and pancreas/prostate). The ‘erythrocyte’ and ‘erythroid progenitor’ annotations were further grouped to minimize multicollinearity.

Using the entire cell type space spanning all four organ compartments, either 30 observations (for example, measured cells) were randomly sampled or the maximum number of available observations (if less than 30) was subsampled, whichever was greater.

Cell type annotations were then reassigned based on the ‘broader’ categories from hierarchical clustering (‘coarsegrain.py’). Raw count values from the DecontX adjusted layer were used to minimize signal spread contamination that could affect DEG analysis (The Tabula Sapiens Consortium and Quake 2021).

This subsampled counts matrix was then passed to the ‘Create Signature Matrix’ analysis module at www.cibersortx.stanford.edu, with the following parameters:
Disable quantile normalization=True
Minimum expression=0.25
Replicates=5
Sampling=0.5
Kappa=999
q value=0.01
No. of barcode genes=3,000-5,000
Filter non-hematopoietic genes=False

The resulting basis matrix was used in the nu-SVR deconvolution code, available on GitHub, under the name ‘tsp_v1_basisMatrix.txt’.

Abbreviations (left) of grouped cell types (right) in the figures are as follows:
gland cell: ‘acinar cell of salivary gland/myoepithelial cell’
respiratory ciliated cell: ‘ciliated cell/lung ciliated cell’
prostate epithelia: ‘club cell of prostate epithelium/hillock cell of prostate epithelium/hillock-club cell of prostate epithelium’
salivary/bronchial secretory cell: ‘duct epithelial cell/serous cell of epithelium of bronchus’
intestinal enterocyte: ‘enterocyte of epithelium of large intestine/enterocyte of epithelium of small intestine/intestinal crypt stem cell of large intestine/large intestine goblet cell/mature enterocyte/paneth cell of epithelium of large intestine/small intestine goblet cell’
intestinal crypt stem cell: ‘immature enterocyte/intestinal crypt stem cell/intestinal crypt stem cell of small intestine/transit amplifying cell of large intestine’
erythrocyte/erythroid progenitor: ‘erythrocyte/erythroid progenitor’
fibroblast/mesenchymal stem cell: ‘fibroblast/mesenchymal stem cell’
intestinal secretory cell: ‘intestinal enteroendocrine cell/paneth cell of epithelium of small intestine/transit amplifying cell of small intestine’
ionocyte/luminal epithelial cell of mammary gland: ‘ionocyte/luminal epithelial cell of mammary gland’
secretory cell: ‘mucus secreting cell/secretory cell/tracheal goblet cell’
pancreatic alpha/beta cell: ‘pancreatic alpha cell/pancreatic beta cell’
respiratory secretory cell: ‘respiratory goblet cell/respiratory mucous cell/serous cell of epithelium of trachea’
basal prostate cell: ‘basal cell of prostate epithelia’

### Nu-SVR Deconvolution

The cell-free transcriptome was formulated as a linear summation of the cell types from which it originates^{3,87}. With this formulation, existing deconvolution methods developed with the objective of decomposing a bulk tissue sample into its single-cell constituents-^{52,53} was adapted, where the deconvolution problem is formulated as:

Aθ=b (3)

Here, A is the representative basis matrix (g×c) of g genes for c cell types, which represent the gene expression profiles of the c cell types. θ is a vector (c×1) of the contributions of each of the cell types, and b is the measured expression of the genes observed in blood plasma (g×1). The goal here is to learn θ such that the matrix product Aθ predicts the measured signal b. The derivation of the basis matrix A is described in the section ‘Basis matrix formation’.

Nu-SVR was performed using a linear kernel to learn θ from a subset of genes from the basis matrix to best recapitulate the observed signal b, where nu corresponds to a lower bound on the fraction of support vectors and an upper bound on the fraction of margin eirors⁸⁸. Here, the support vectors are the genes from the basis matrix used to learn θ; θ reflects the learned weights of the cell types in the basis matrix column space. For each sample, a set of 0 was learned by performing a grid search on the two SVR hyperparameters: v∈{0.05, 0.1, 0.15, 0.25, 0.5, 0.75} and C∈{0.1,0.5,0.75, 1, 10}.

For each sample, two constraints were enforced: θ can contain only non-negative weights, and the weights in θ must sum to 1. Each θ corresponding to a hyperparameter combination was normalized as previously described in two steps^{52,53} First, only non-negative weights were kept:

∀θj<0∈{θ₁, . . . ,θ_{c}}→0 (4)

Second, the remaining non-zero weights were then normalized by their sum to yield the relative proportions of cell-type-specific RNA.

The basis matrix dot product was determined with the set of normalized weights for each sample. This dot product yields the predicted expression value for each gene in a given cfRNA mixture with imposed non-negativity on the normalized coefficient vector. The root mean square error (RMSE) was then computed using the predicted expression values and the measured values of these genes for each hyperparameter combination in a given cfRNA mixture. The model yielding the smallest RMSE in predicting expression for a given cfRNA sample was then chosen and assigned as the final deconvolution result for a given sample.

Only CPM counts≥1 were considered in the mixture, b. The values in the basis matrix were also CPM normalized. Before deconvolution, the mixture and basis matrix were centered and scaled to zero mean and unit variance for improved runtime performance. Counts were not log-transformed in b or in A, as this would destroy the requisite linearity assumption in equation (3). Specifically, the concavity of the log function would result in the consistent underestimation of θ during deconvolution⁸⁹.

The function nu-SVR from scikitlearn⁹⁰ version 0.23.2 was used.

The samples used for nu-SVR deconvolution were 75 NCI patients from Toden et al. spanning four sample collection centers. Given center-specific batch effects reported by Toden et al., results herein are reported on a per-center basis (FIG. 1d and FIGS. 6 and 7). There was good pairwise similarity of the learned coefficients among biological replicates within and across sample centers (FIG. 7a,b). Deconvolution performance yielded RMSE and Pearson r consistent with deconvolved GTEx tissues (FIG. 5) whose distinct cell types were in the basis matrix column space (FIG. 7c,d). In interpreting the resulting cell type fractions, a limitation of nu-SVR is that it uses highly expressed genes as support vectors and, consequently, assigns a reduced fractional contribution to cell types expressing genes at lower levels or that are smaller in cell volume. Comparison of nu-SVR to quadratic programming³ and non-negative linear least squares⁹¹ yielded similar deconvolution RMSE and Pearson correlation. In contrast to the other methods, nu-SVR cell type contributions were the most consistent with the cell type markers detected using PanglaoDB and was, hence, chosen as the deconvolution model for this work.

### Evaluating Basis Matrix on GTEx Samples

Bulk RNA sequencing samples from GTEx version 8 were deconvolved with the derived basis matrix from tissues that were present (that is, kidney cortex, whole blood, lung and spleen) or absent (for example, kidney medulla and brain) from the basis matrix derived using Tabula Sapiens version 1.0. For each tissue type, the maximum number of available samples or 30 samples, whichever was smaller, was deconvolved.

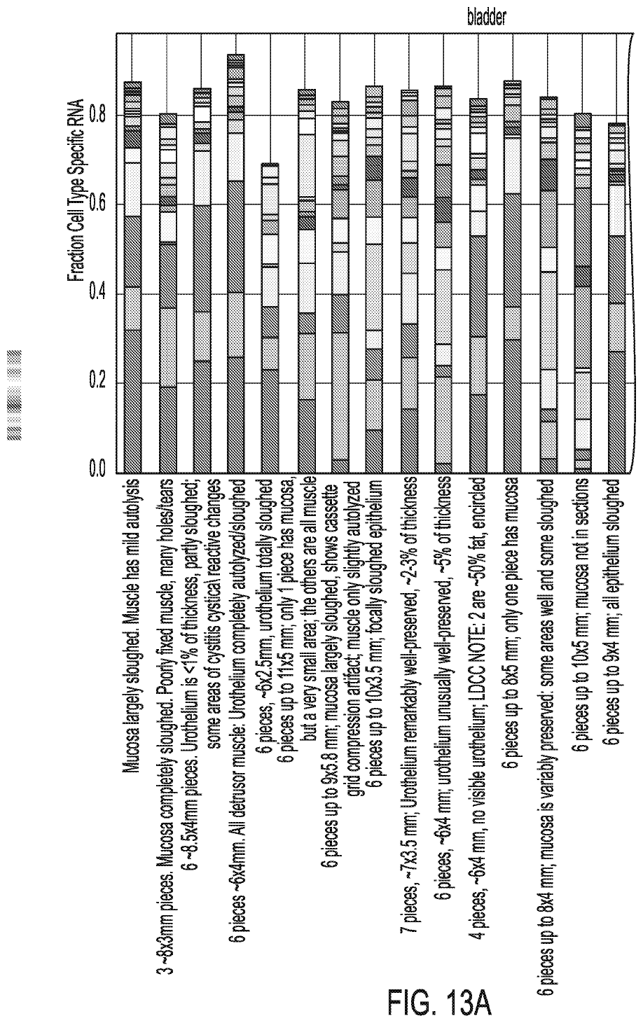

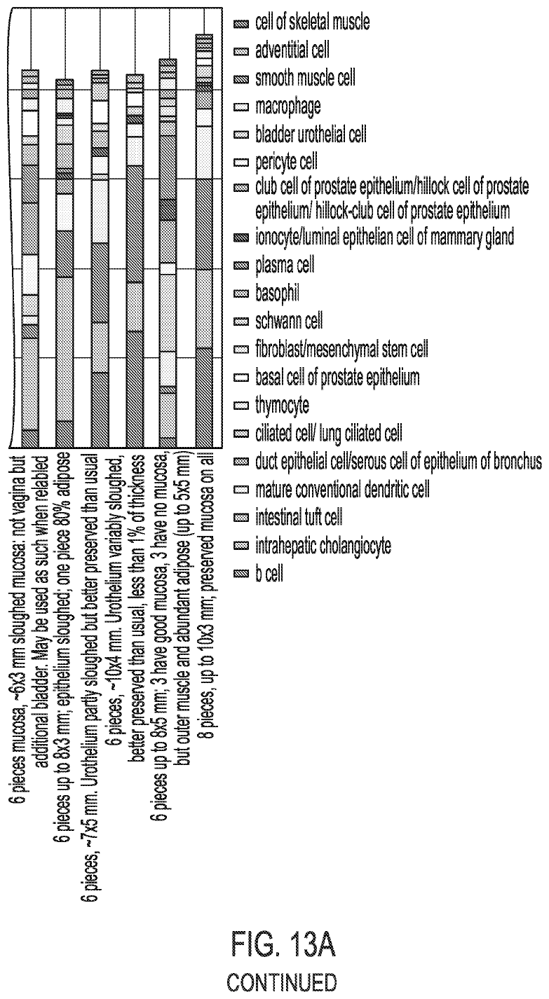

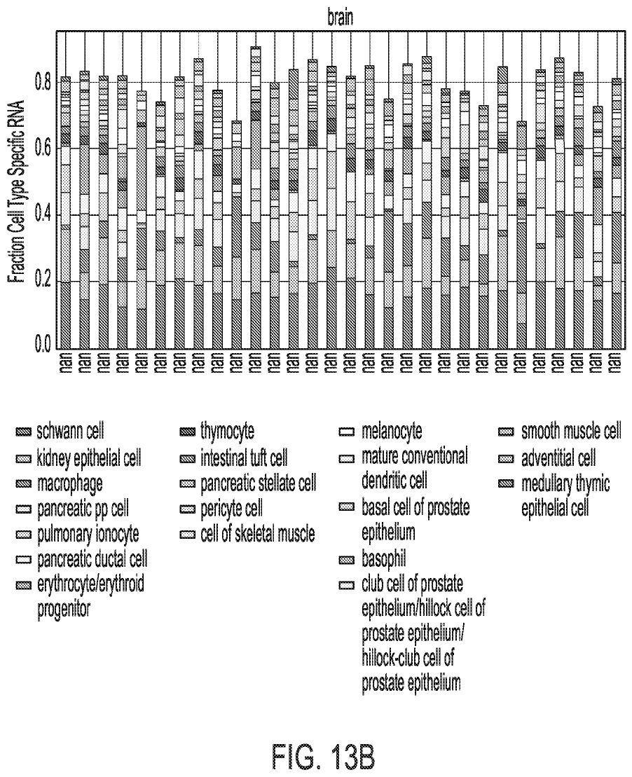

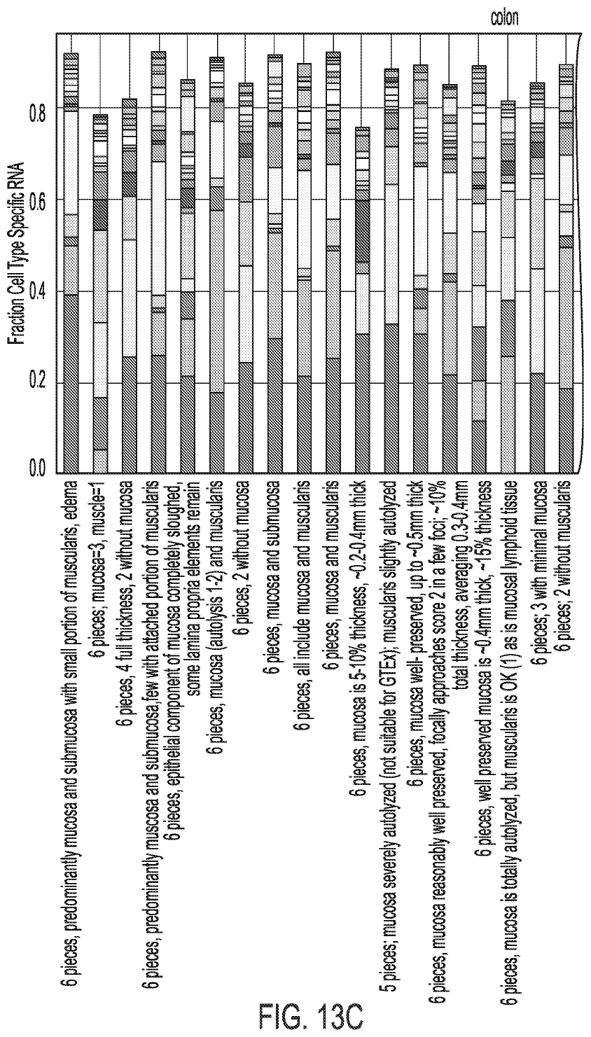

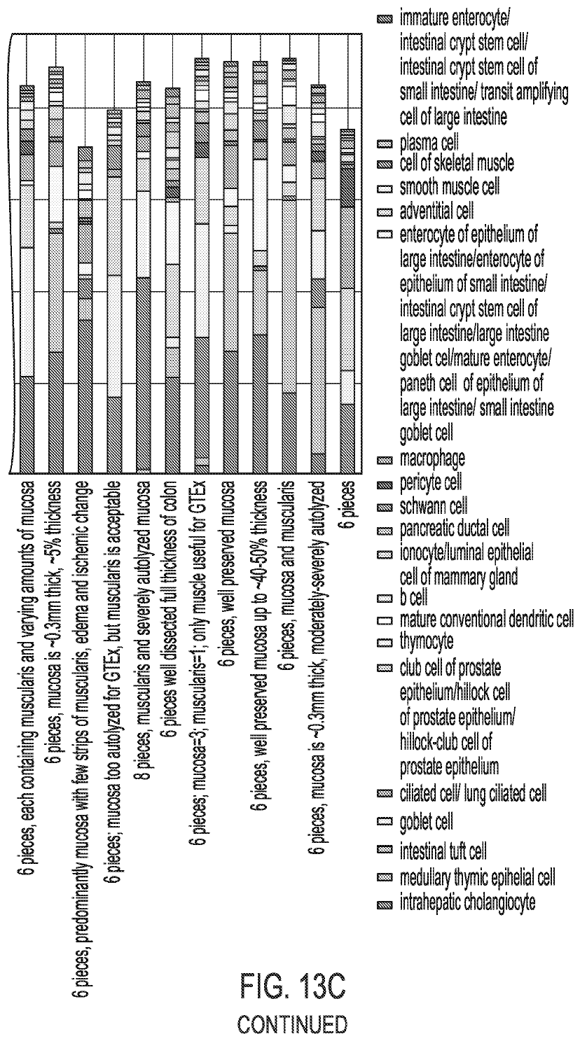

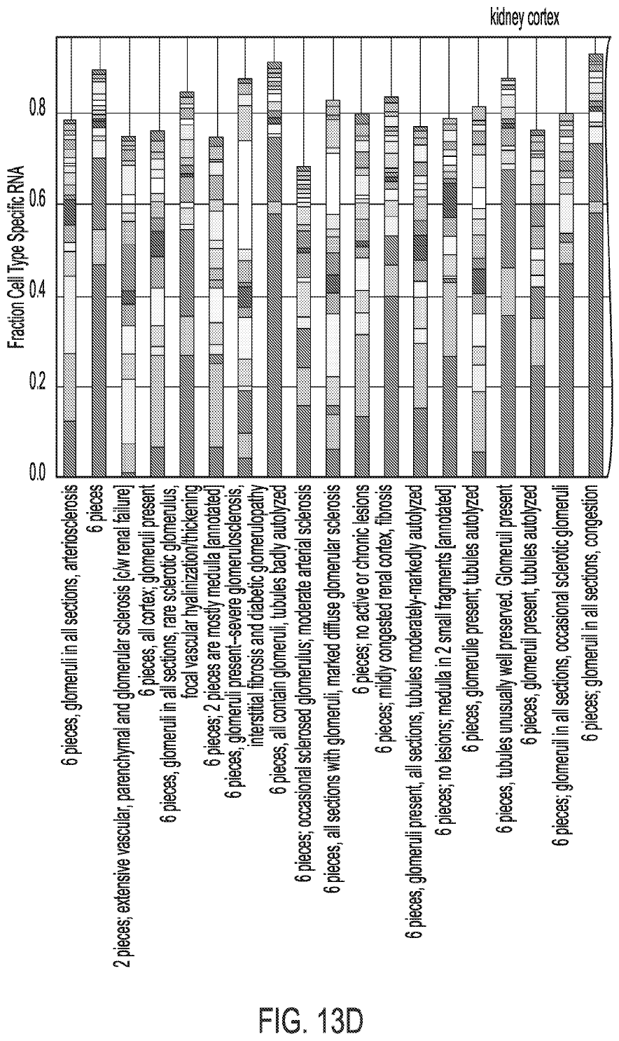

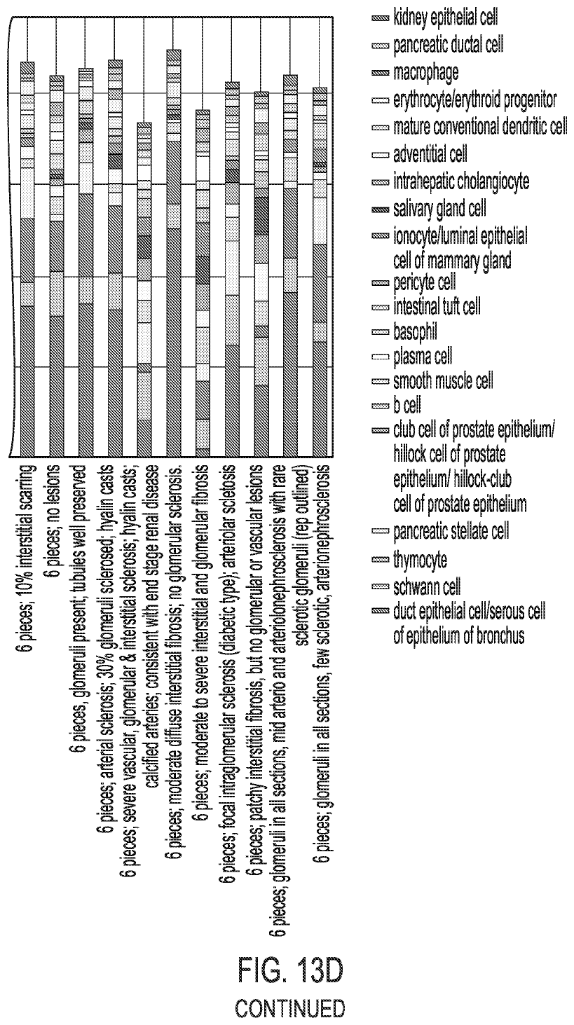

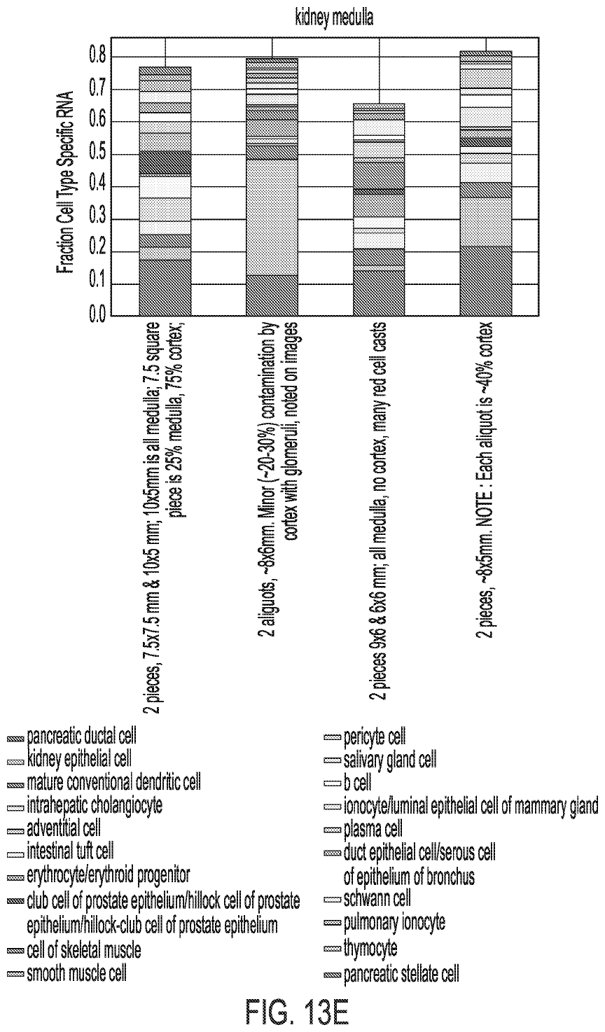

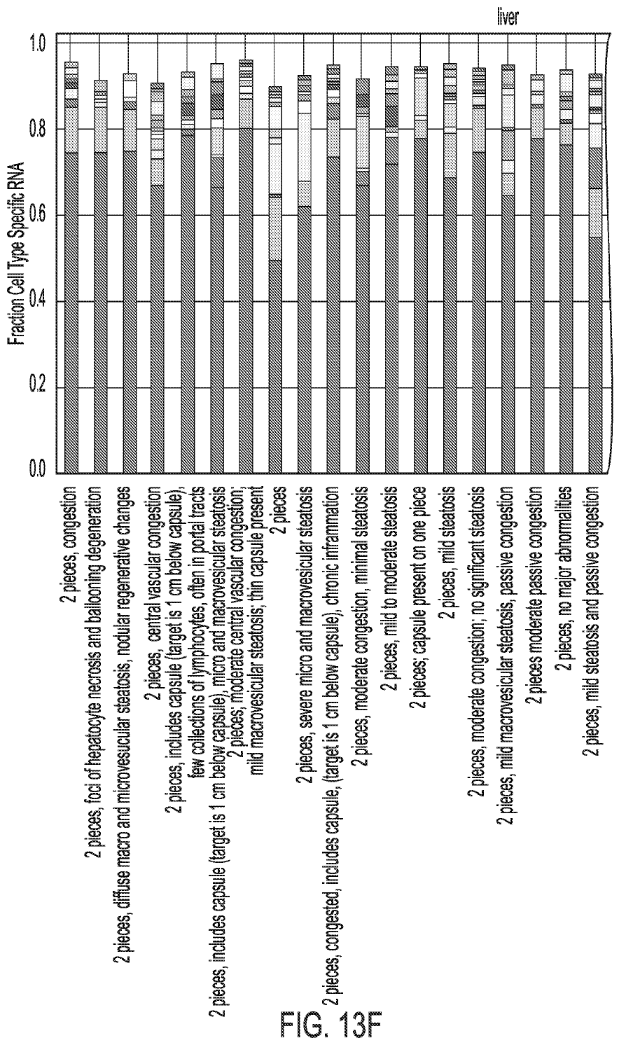

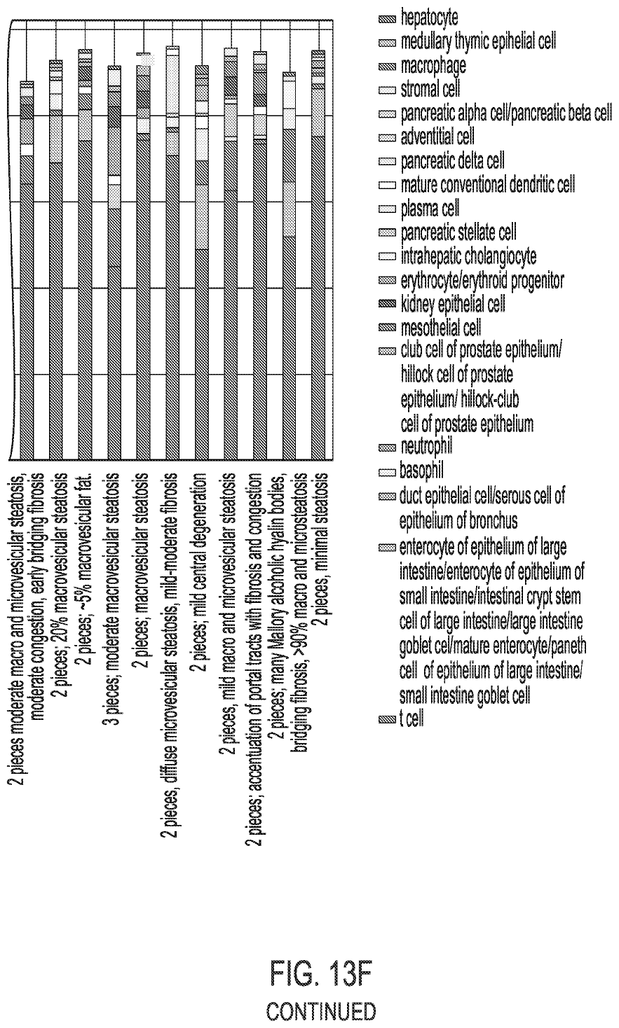

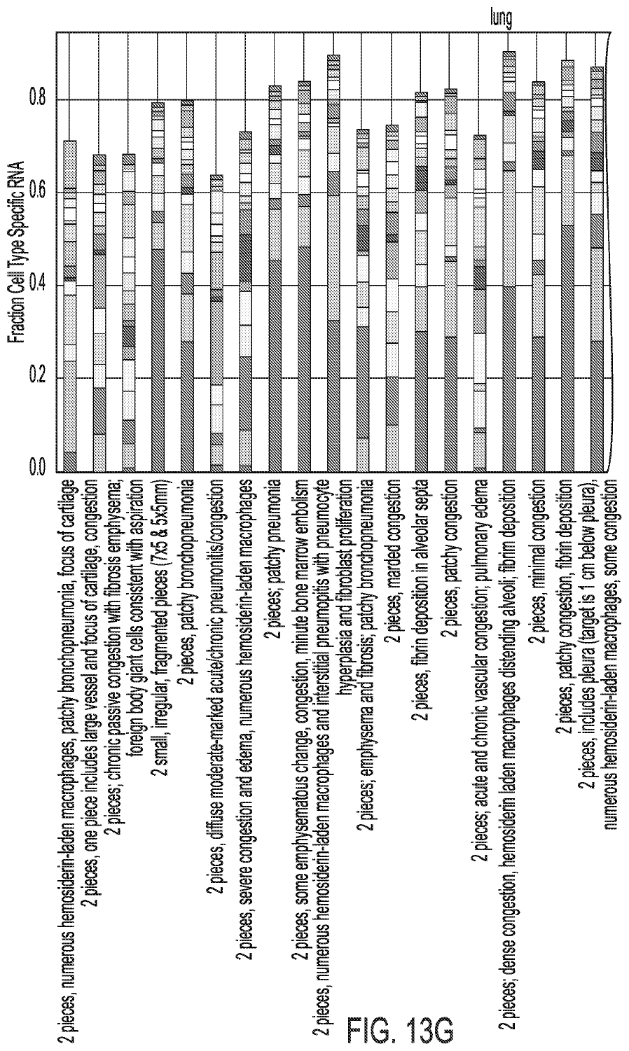

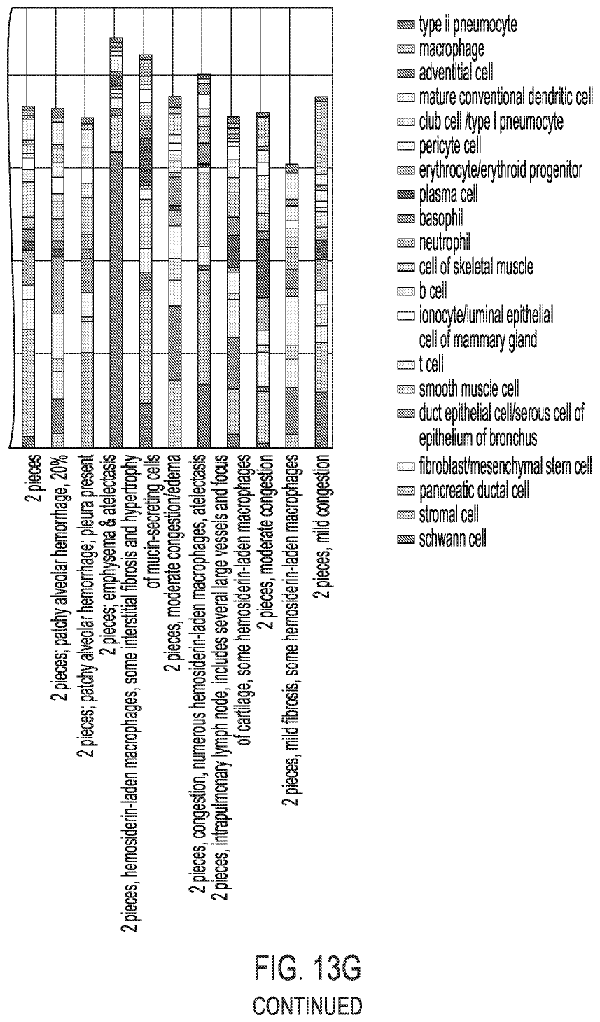

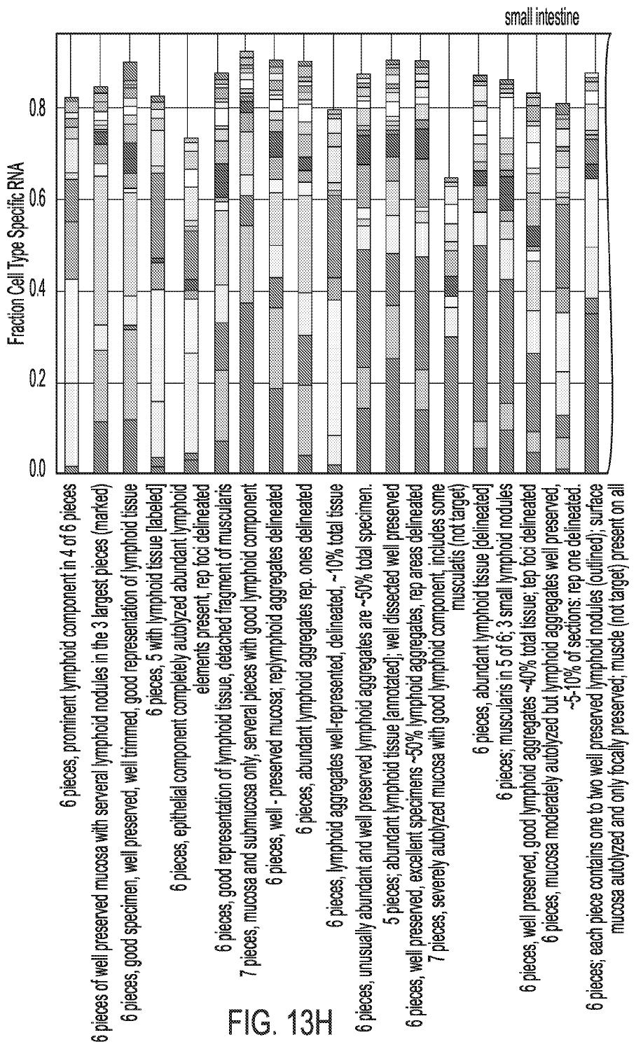

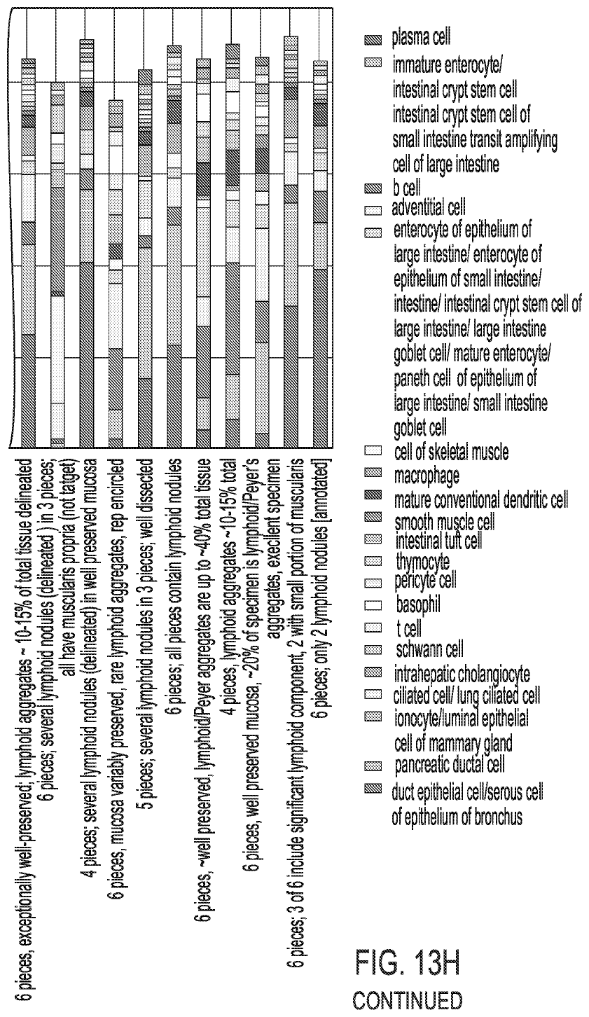

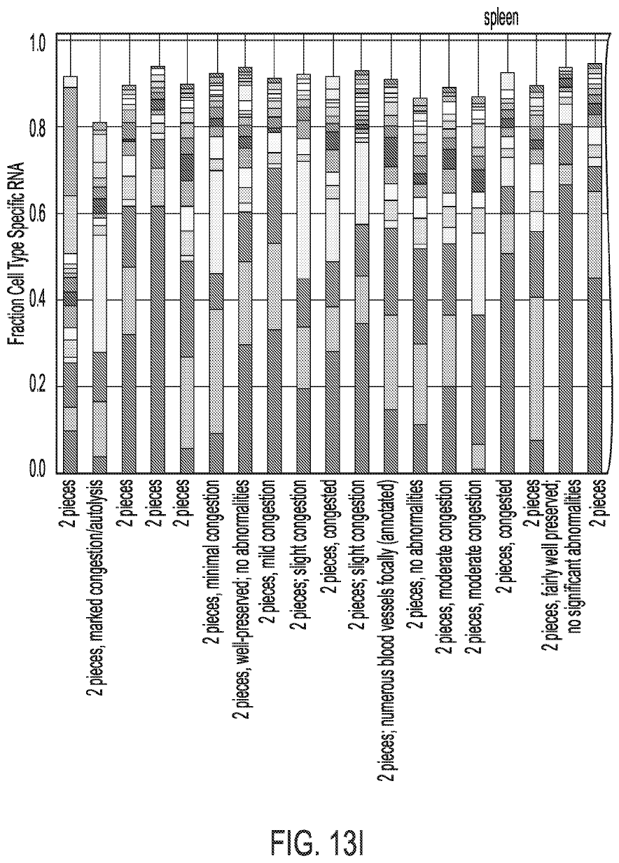

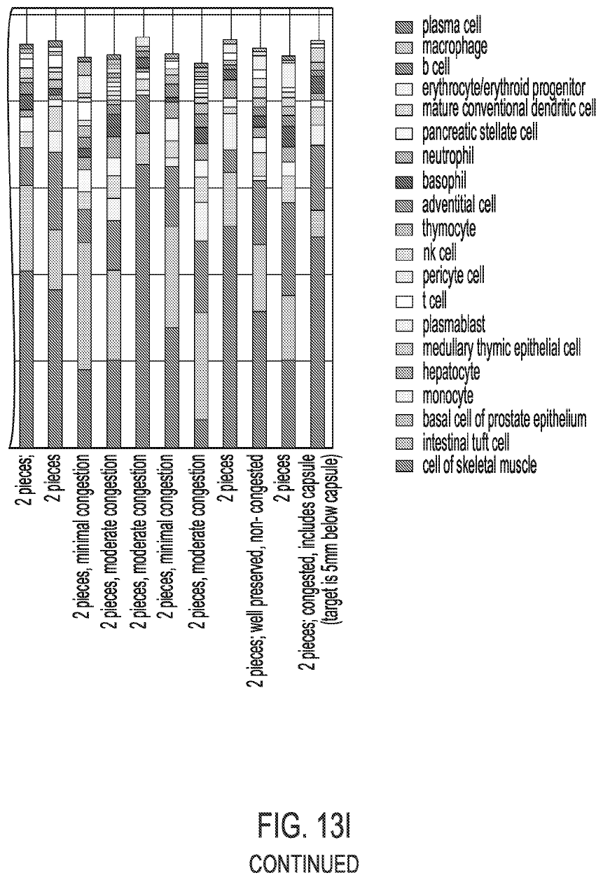

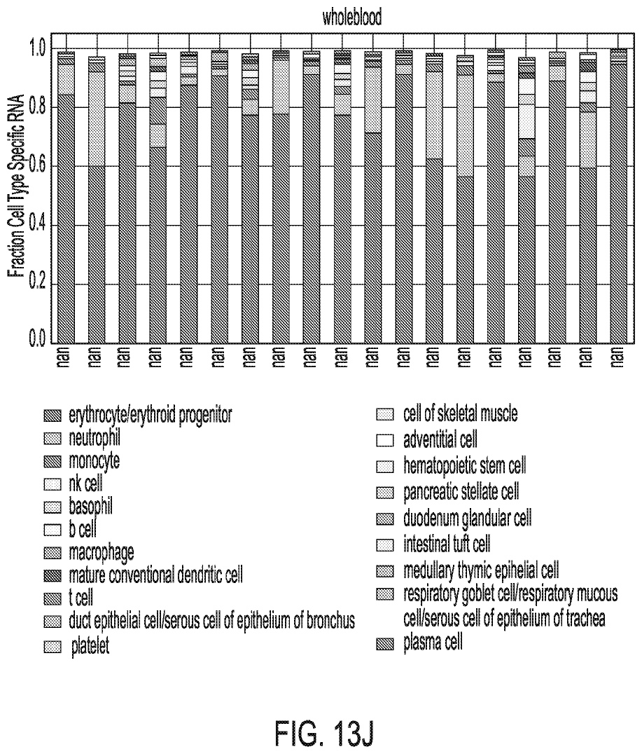

To assess the ability of the basis matrix to deconvolve tissues whose cell types were wholly present in the cell type column space, a subset of bulk RNA-seq GTEx samples was deconvolved. The determined fractions of cell type specific RNA recapitulated the predominant cell types within a given tissue (FIG. 13). Organs with increased cell type heterogeneity (lung, bladder, kidney, intestine, colon) in contrast to tissues with reduced spatial heterogeneity (liver, spleen, whole blood)1, exhibited greater variance in deconvolved fractions (FIG. 13) and deconvolution performance (FIG. 5). Tissues with reduced spatial heterogeneity whose cell types were wholly in the basis matrix column space include predominantly b cells/plasma cells and erythrocytes in spleen; hepatocytes, liver; erythrocytes and leukocytes, whole blood. Cell types belonging to tissues with increased spatial heterogeneity exhibited greater variance in deconvolved fractions: kidney cortex majority fractions were from kidney epithelia and lymphocytes; small intestine, intestinal enterocytes and lymphocytes; lung, pneumonocytes and immune cells, colon, intestinal enterocytes, lymphocytes, and muscle cells. Cells with larger volume yielded larger deconvolved fractions across all tissues. Variance in the relative cell type fractional contributions across the deconvolved bulk samples within a given tissue reflects the underlying cell type heterogeneity, particularly in these complex samples. GTEx kidney medulla samples recorded to be contaminated with renal cortex reflect the presence of the kidney epithelia, the majority cell type in the renal cortex. Given that the kidney medulla is not part of TSP v1.0, high deconvolution performance was not expected since its cell types are absent from the basis matrix column space. The brain, whose cell types were wholly absent from the cell type column space exhibited poor deconvolution performance, as expected. However, the majority cell type fraction assigned was to the cell type belonging to the peripheral nervous system that was present in Tabula Sapiens version 1, the schwann cell, underscoring the ability of the deconvolution method to assign fractional contributions to similar cell types from those that are absent from the basis matrix column space.

Identifying Tissue-Specific Genes in cfRNA Absent from Basis Matrix

To identify cell-type-specific genes in cfRNA that were distinct to a given tissue, the set difference of the non-zero genes measured in a given cfRNA sample was considered with the row space of the basis matrix and intersected this with HPA tissue-specific genes:

(Gj−R)∩HPA (5)
where Gⱼ is the gene set in the j^{th} deconvolved sample, where a given gene in the set's expression was ≥1 CPM. R is the set of genes in the row space of the basis matrix used for nu-SVR deconvolution. HPA denotes the total set of tissue-specific genes from HPA.

The HPA tissue-specific gene set (HPA) comprised genes across all tissues with Tissue Specificity assignments ‘Group Enriched’, ‘Tissue Enhanced’, ‘Tissue Enriched’ and NX expression≥10. This approach yielded tissues with several distinct genes present in cfRNA, which could then be subsequently interrogated using single-cell data.

### Derivation of Cell-Type-Specific Gene Profiles in Context of the Whole Body Using Single-Cell Data

For this analysis, only cell types unique to a given tissue (that is, hepatocytes unique to the liver or excitatory neurons unique to the brain) were considered so that bulk transcriptomic data could be used to ensure specificity in context of the whole body. A gene was asserted to be cell type specific if it was (1) differentially expressed within a given single-cell tissue atlas, (2) possessed a Gini coefficient≥0.6 and was listed as specific to the native tissue for the cell type of interest, indicating comprehensive tissue specificity in context of the whole body (FIGS. 8 and 10).

### (1) Single-Cell Differential Expression

For data received as a Seurat object, conversion to AnnData (version 0.7.4) was performed by saving as an intermediate loom object (Seurat version 3.1.5) and converting to AnnData (loompy version 3.0.6). Scanpy (version 1.6.0) was used for all other single-cell analysis. Reads per cell were normalized for library size (scanpy normalize_total, target_sum=1×10⁴) and then logged (scanpy log 1p). Differential expression was performed using the Wilcoxon rank-sum test in Scanpy's filter_rank_genes_groups with the following arguments: min_fold_change=1.5, min_in_group_fraction=0.2, max_out_group_fraction=0.5, corr_method=‘benjamini-hochberg’. The set of resulting DEGs with Benjamini-Hochberg-adjusted P values<0.01 whose ratio of the highest out-group percent expressed to in-group percent expressed<0.5 was selected to ensure high specific expression in the cell type of interest within a given cell type atlas.

### (2) Quantifying Comprehensive Whole-Body Tissue Specificity Using the Gini Coefficient

The distribution of all the Gini coefficiets and Tau values across all genes belonging to cell type gene profiles for cell types native to a given tissue were compared using the HPA gene expression Tissue Specificity and Tissue Distribution assignments⁵⁰ (FIG. 9). The Gini coefficient better reflected the underlying distribution of gene expression tissue specificity than Tau (FIG. 9) and, hence, were used for subsequent analysis. As the Gini coefficient approaches unity, this indicates extreme gene expression inequality or equivalently high specificity. A single threshold (Gini coefficient≥0.6) was applied across all atlases to facilitate a generalizable framework from which to define tissue-specific cell type gene profiles in context of the whole body in a principled fashion for signature scoring in cfRNA.

For the following definitions, n denotes the total number of tissues, and xⱼ is the expression of a given gene in the i^{th} tissue.

The Gini coefficient was computed as defined⁵⁹:

$$Gini = \frac{n + 1}{n} - \frac{2 {\sum }_{i = 1}^{n} ( n + 1 - i ) {x}_{i}}{n {\sum }_{i = 1}^{n} {x}_{i}} ;$$

xᵢ is ordered from least to greatest.

Tau, as defined in ref.⁵⁹:

$$τ = \frac{{\sum }_{i = 1}^{n} 1 - \overline{x}}{n - 1} where \overline{x} = \frac{{x}_{i}}{max ( {x}_{i} ) ∀ i ∈ \{ 1 … n \}} \qquad ( 7 )$$

HPA NX Counts from the HPA object titled ‘ma_tissue_consensus.tsv’ accessed on 1 Jul. 2019 were used for computing Gini coefficients and Tau.

Note for brain cell type gene profiles: Given that there are multiple sub brain regions in the HPA data, the determined Gini coefficients are lower (for example, not as close to unity compared to other cell type gene profiles) because there are multiple regions of the brain with high expression, which would result in reduced count inequality.

### Gene Expression in GTEx

The specificity of a given gene profile to its corresponding cell type was confirmed by comparing the aggregate expression of a given cell type signature in its native tissue compared to that of the average across remaining GTEx tissues (FIGS. 8d and 10f,g). A median fold change was uniformly observed greater than 1 in the signature score of a cell type gene profile in its native tissue relative to the mean expression in other tissues, confirming high specificity.

Raw GTEx data version 8 (accessed 26 Aug. 2019) were converted to log(counts-per-ten-thousand+1) counts. The signature score was determined by summing the expression of the genes in a given bulk RNA sample for a given cell type gene profile. Because only gene profiles were derived for cell types that correspond to a given tissue, the mean signature score of a cell type profile across the non-native tissues was then computed and used to determine the log fold change.

Cell Type Specificity of DEGs in AD and NAFLD cfRNA

After observing a significant intersection between the DEGs in AD4 (Toden et al. 2020) or NAFLD5 in cfRNA with corresponding cell-type-specific genes (FIG. 12c,e), the cell type specificity of DEGs was next assessed using a permutation test. To assess whether DEGs that intersected with a cell type gene profile were more specific to a given cell type than DEGs that were generally tissue specific, a permutation test was performed. Specifically, the Gini coefficient for genes in these two groups were compared, computed using the mean expression of a given gene across brain cell types from healthy brain10 or liver55 single-cell data. The cell type gene profiles were considered as defined for signature scoring in FIG. 2.

The starting set of tissue-specific genes was defined using the HPA tissue transcriptional data annotated as ‘Tissue enriched’, ‘Group enriched’ or ‘Tissue enhanced’ (brain, accessed on 13 Jan. 2021; liver, accessed on 28 Nov. 2020). These requirements ensured the specificity of a given brain/liver gene in context of the whole body. For a given tissue, this formed the initial set of tissue-specific genes B.

The union of all brain or liver cell-type-specific genes is the set C. All genes in C (‘cell type specific’) were a subset of the respective initial set of tissue-specific genes:

C−B={ } (8)

Genes in B that did not intersect with C and intersected with DEG-up (U) or DEG-down (D) genes in a given disease^{4,5} were then defined as ‘tissue specific’.

T=(B∩U)U(B∩D)−C (9)

The Gini coefficients reflecting the gene expression inequality across the cell types within corresponding tissue single-cell atlas were computed for the gene sets labeled as ‘cell type specific’ and ‘tissue specific’. Brain reference data to compute Gini coefficients were from the single-cell brain atlas with diagnosis as ‘Normal’¹⁰. Liver single cell data were used as-is⁵. All Gini coefficients were computed using the mean log-transformed CPFTT (counts per ten thousand) gene expression per cell type.

A permutation test was then performed on the union of the Gini coefficients for the genes labeled as ‘cell type specific’ and ‘tissue specific’. The purpose of this test was to assess probability that the observed mean difference in Gini coefficient for these two groups yielded no difference in specificity (that is, H₀: μ_{cell type Gini coefficient}=μ_{tissue Gini coeffcient}).

Gini coefficients were permuted and reassigned to the list of ‘tissue specific’ or ‘cell type specific’ genes, and then the difference in the means of the two groups was computed. This procedure was repeated 10,000 times. The P value was determined as follows:

$$p = \frac{\# trials with permuted ( {μ}_{cell type} - {μ}_{tissue} ) \geq {μ}_{observed}}{10 , 000 + 1} \qquad ( 10 )$$

$$where$$

$${μ}_{observed} := ( {μ}_{cell type Gini Coefficient} - {μ}_{tissue Gini coeffieint} ) .$$

The additional 1 in the denominator reflects the original test between the true difference in means (the true comparison yielding μ_{observed}).

NAFLD: The space of reported NAFLD DEGs in serum⁵ was considered. Here, C=hepatocyte gene profile, and B=the liver-specific genes.

AD: First, a given cell type gene profile in AD was intersected with the equivalent Normal profile for comparative analysis. Genes defined as ‘brain cell type specific’ for signature scoring in FIG. 2d were used in this comparison. Of note, no DEG-up genes intersected with any of the brain cell type signatures in FIG. 2d. Microglia, although often implicated in AD pathogenesis, were excluded given their high overlapping transcriptional profile with non-central-nervous-system macrophages⁹². Inhibitory neurons were also excluded given the low number of cell-type-specific genes intersecting between AD and NCI phenotypes.

### Estimating Signature Scores for Each Cell Type

The signature score is defined as the sum of the log-transformed CPM-TMM normalized counts per gene asserted to be cell type specific, where i denotes the index of the gene in a cell type signature gene profile G in the j^{th} patient sample:

Signature Scoreⱼ=ΣᵢGᵢⱼ (11)

### Preeclampsia

For signature scoring of syncytiotrophoblast and extravillous trophoblast gene profiles in PEARL-PEC and iPEC⁴⁶, a respective cell type gene profile used for signature scoring was derived as described in ‘Derivation of cell-type-specific gene profiles in context of the whole body using single-cell data’ independently using two different placental single-cell datasets^{56,57}. Only the intersection of the cell-type-specific gene profiles for a given trophoblast cell type between the two datasets was included in the respective trophoblast gene profile for signature scoring.

### CKD

The signature score of the proximal tubule in CKD (nine patients; 51 samples) and healthy controls (three patients; nine samples) was compared. Given that all patient samples were longitudinally sampled over ˜30 d (individual samples were taken on different days), the samples were treated as biological replicates and included all time points because the time scale over which renal cell type changes typically occur is longer than the collection period. The sequencing depth was similar between the CKD and healthy cohorts, although it was reduced in comparison to the other cfRNA datasets used in this work. To account for gene measurement dropout, the expression of a given gene in the proximal tubule gene profile was required to be non-zero in at least one sample in both cohorts. Given that all samples were sequenced together, no batch correction was necessary, facilitating a representative comparison between CKD and healthy cohorts.

### AD

Microglia, although often implicated in AD pathogenesis, were excluded given their high overlapping transcriptional profile with non-central-nervous-system macrophages⁹². Inhibitory neurons were also excluded given the low number of cell-type-specific genes intersecting between AD and NCI phenotypes. Brain gene profiles as defined in the AD section of ‘Cell type specificity of DEGs in AD and NAFLD cfRNA’ were used.

### Assessing P Value Calibration for a Given Signature Score

Cell type signature scores were tested between control and diseased samples with a Mann-Whitney U-test. The resulting P values were calibrated with a permutation test. Here, the labels compared in a given test (that is, CKD versus control, AD versus NCI, NAFLD versus control, etc.) were randomly shuffled 10,000 times. A well-calibrated, uniform P-value distribution was observed (FIG. 12a), validating the experimentally observed test statistics.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

### REFERENCES

1. Ibarra, A. et al. Non-invasive characterization of human bone marrow stimulation and reconstitution by cell-free messenger RNA sequencing. Nat. Commun. 11, 400 (2020).
2. Ngo, T. T. M. et al. Noninvasive blood tests for fetal development predict gestational age and preterm delivery. Science 360, 1133-1136 (2018).
3. Koh, W. et al. Noninvasive in vivo monitoring of tissue-specific global gene expression in humans. Proc Natl Acad Sci USA 111, 7361-7366 (2014).
4. Toden, S. et al. Noninvasive characterization of Alzheimer's disease by circulating, cell-free messenger RNA next-generation sequencing. Sci. Adv. 6, (2020).
5. Chalasani, N. et al. Noninvasive stratification of nonalcoholic fatty liver disease by whole transcriptome cell-free mRNA characterization. Am. J. Physiol. Gastrointest. Liver Physiol. 320, G439-G449 (2021).
6. Klatt, E. C. Robbins & Cotran Atlas of Pathology. (Elsevier, 2021).
7. Sin, M. L. Y. et al. Deep sequencing of urinary rnas for bladder cancer molecular diagnostics. Clin. Cancer Res. 23, 3700-3710 (2017).
8. Bryois, J. et al. Genetic identification of cell types underlying brain complex traits yields insights into the etiology of Parkinson's disease. Nat. Genet. 52, 482-493 (2020).
9. Urbanska, K., Sokolowska, J., Szmidt, M. & Sysa, P. Glioblastoma multiforme—an overview. Contemp Oncol (Pozn) 18, 307-312 (2014).
10. Mathys, H. et al. Single-cell transcriptomic analysis of Alzheimer's disease. Nature 570, 332-337 (2019).
11. Hannan, R. D., Jenkins, A., Jenkins, A. K. & Brandenburger, Y. Cardiac hypertrophy: a matter of translation. Clin. Exp. Pharmacol. Physiol. 30, 517-527 (2003).
12. Wu, Q.-Q. et al. Mechanisms contributing to cardiac remodelling. Clin. Sci. 131, 2319-2345 (2017).
13. Chiong, M. et al. Cardiomyocyte death: mechanisms and translational implications. Cell Death Dis. 2, e244 (2011).
14. Harvey, P. A. & Leinwand, L. A. The cell biology of disease: cellular mechanisms of cardiomyopathy. J. Cell Biol. 194, 355-365 (2011).
15. Lazzara, R., El-Sherif, N., Hope, R. R. & Scherlag, B. J. Ventricular arrhythmias and electrophysiological consequences of myocardial ischemia and infarction. Circ. Res. 42, 740-749 (1978).
16. Schumann, M., Siegmund, B., Schulzke, J. D. & Fromm, M. Celiac disease: role of the epithelial barrier. Cell. Mol. Gastroenterol. Hepatol. 3, 150-162 (2017).
17. Worthington, J. J., Reimann, F. & Gribble, F. M. Enteroendocrine cells-sensory sentinels of the intestinal environment and orchestrators of mucosal immunity. Mucosal Immunol. 11, 3-20 (2018).
18. Ciccocioppo, R. et al. Increased enterocyte apoptosis and Fas-Fas ligand system in celiac disease. Am. J. Clin. Pathol. 115, 494-503 (2001).
19. Wehkamp, J. & Stange, E. F. An update review on the paneth cell as key to ileal crohn's disease. Front. Immunol. 11, 646 (2020).
20. Di Sabatino, A. et al. Increased enterocyte apoptosis in inflamed areas of Crohn's disease. Dis. Colon Rectum 46, 1498-1507 (2003).
21. Yu, Y., Yang, W., Li, Y. & Cong, Y. Enteroendocrine cells: sensing gut microbiota and regulating inflammatory bowel diseases. Inflamm. Bowel Dis. 26, 11-20 (2020).
22. Gersemann, M., Stange, E. F. & Wehkamp, J. From intestinal stem cells to inflammatory bowel diseases. World J. Gastroenterol. 17, 3198-3203 (2011).
23. Sadanandam, A. et al. A colorectal cancer classification system that associates cellular phenotype and responses to therapy. Nat. Med. 19, 619-625 (2013).
24. Pflügler, S. et al. IDO1+Paneth cells promote immune escape of colorectal cancer. Commun. Biol. 3, 252 (2020).
25. Feldstein, A. E. & Gores, G. J. Apoptosis in alcoholic and nonalcoholic steatohepatitis. Front. Biosci. 10, 3093-3099 (2005).
26. Hammoutene, A. & Rautou, P.-E. Role of liver sinusoidal endothelial cells in non-alcoholic fatty liver disease. J. Hepatol. 70, 1278-1291 (2019).
27. Sorensen, K. K., Simon-Santamaria, J., McCuskey, R. S. & Smedsrød, B. Liver sinusoidal endothelial cells. Compr. Physiol. 5, 1751-1774 (2015).
28. Jindal, A., Thadi, A. & Shailubhai, K. Hepatocellular carcinoma: etiology and current and future drugs. J. Clin. Exp. Hepatol. 9, 221-232 (2019).
29. Braet, F. & Wisse, E. Structural and functional aspects of liver sinusoidal endothelial cell fenestrae: a review. Comp. Hepatol. 1, 1 (2002).
30. Witsch, I. H. Proliferation of type II alveolar cells: a review of common responses in toxic lung injury. Toxicology 5, 267-277 (1976).
31. Olajuyin, A. M., Zhang, X. & Ji, H.-L. Alveolar type 2 progenitor cells for lung injury repair. Cell Death Discov. 5, 63 (2019).
32. Tilley, A. E., Walters, M. S., Shaykhiev, R. & Crystal, R. G. Cilia dysfunction in lung disease. Annu. Rev. Physiol. 77, 379-406 (2015).
33. Travis, W. D. Pathology of lung cancer. Clin. Chest Med. 32, 669-692 (2011).
34. Reiser, J. & Sever, S. Podocyte biology and pathogenesis of kidney disease. Annu. Rev. Med. 64, 357-366 (2013).
35. Schelling, J. R. Tubular atrophy in the pathogenesis of chronic kidney disease progression. Pediatr. Nephrol. 31, 693-706 (2016).
36. Yu, S. M.-W. & Bonventre, J. V. Acute kidney injury and progression of diabetic kidney disease. Adv. Chronic Kidney Dis. 25, 166-180 (2018).
37. Garg, P. A review of podocyte biology. Am. J. Nephrol. 47 Suppl 1, 3-13 (2018).
38. Basile, D. P., Anderson, M. D. & Sutton, T. A. Pathophysiology of acute kidney injury. Compr. Physiol. 2, 1303-1353 (2012).
39. Davidson, A. What is damaging the kidney in lupus nephritis? Nat. Rev. Rheumatol. 12, 143-153 (2016).
40. Haeberle, L. & Esposito, I. Pathology of pancreatic cancer. Transl. Gastroenterol. Hepatol. 4, 50 (2019).
41. Long, R. M., Morrissey, C., Fitzpatrick, J. M. & Watson, R. W. G. Prostate epithelial cell differentiation and its relevance to the understanding of prostate cancer therapies. Clin. Sci. 108, 1-11 (2005).
42. Cheng, L. et al. Testicular cancer. Nat. Rev. Dis. Primers 4, 29 (2018).
43. Li, H., Huang, S., Guo, C., Guan, H. & Xiong, C. Cell-free seminal mRNA and microRNA exist in different forms. PLoS ONE 7, e34566 (2012).
44. Larson, M. H. et al. A comprehensive characterization of the cell-free transcriptome reveals tissue- and subtype-specific biomarkers for cancer detection. Nat. Commun. 12, 2357 (2021).
45. Ngo, T. T. M., Moufarrej, M. N. & Rasmussen, M. L. H. Noninvasive blood tests for fetal development predict gestational age and preterm delivery. science.sciencemag.org.
46. Munchel, S. et al. Circulating transcripts in maternal blood reflect a molecular signature of early-onset preeclampsia. Sci. Transl. Med. 12, (2020).
47. Gawad, C., Koh, W. & Quake, S. R. Single-cell genome sequencing: current state of the science. Nat. Rev. Genet. 17, 175-188 (2016).
48. The Tabula Sapiens Consortium & Quake, S. R. The Tabula Sapiens: a single cell transcriptomic atlas of multiple organs from individual human donors. BioRxiv (2021) doi:10.1101/2021.07.19.452956.
49. GTEx Consortium et al. Genetic effects on gene expression across human tissues. Nature 550, 204-213 (2017).
50. Uhlen, M. et al. A genome-wide transcriptomic analysis of protein-coding genes in human blood cells. Science 366, (2019).
51. Franzén, O., Gan, L.-M. & Björkegren, J. L. M. PanglaoDB: a web server for exploration of mouse and human single-cell RNA sequencing data. Database (Oxford) 2019, (2019).
52. Newman, A. M. et al. Determining cell type abundance and expression from bulk tissues with digital cytometry. Nat. Biotechnol. 37, 773-782 (2019).
53. Newman, A. M. et al. Robust enumeration of cell subsets from tissue expression profiles. Nat. Methods 12, 453-457 (2015).
54. Sadeh, R. et al. ChIP-seq of plasma cell-free nucleosomes identifies gene expression programs of the cells of origin. Nat. Biotechnol. 39, 586-598 (2021).
55. Aizarani, N. et al. A human liver cell atlas reveals heterogeneity and epithelial progenitors. Nature 572, 199-204 (2019).
56. Suryawanshi, H. et al. A single-cell survey of the human first-trimester placenta and decidua. Sci. Adv. 4, eaau4788 (2018).
57. Vento-Tormo, R. et al. Single-cell reconstruction of the early maternal-fetal interface in humans. Nature 563, 347-353 (2018).
58. Stewart, B. J. et al. Spatiotemporal immune zonation of the human kidney. Science 365, 1461-1466 (2019).
59. Kryuchkova-Mostacci, N. & Robinson-Rechavi, M. A benchmark of gene expression tissue-specificity metrics. Brief Bioinformatics 18, 205-214 (2017).
60. András, I. E. & Toborek, M. Extracellular vesicles of the blood-brain barrier. Tissue Barriers 4, e1131804 (2016).
61. Abbott, N. J. Inflammatory mediators and modulation of blood-brain barrier permeability. Cell. Mol. Neurobiol. 20, 131-147 (2000).
62. Ganong, W. F. Circumventricular organs: definition and role in the regulation of endocrine and autonomic function. Clin. Exp. Pharmacol. Physiol. 27, 422-427 (2000).
63. Tsang, J. C. H. et al. Integrative single-cell and cell-free plasma RNA transcriptomics elucidates placental cellular dynamics. Proc Natl Acad Sci USA 114, E7786-E7795 (2017).
64. Kaufmann, P., Black, S. & Huppertz, B. Endovascular trophoblast invasion: implications for the pathogenesis of intrauterine growth retardation and preeclampsia. Biol. Reprod. 69, 1-7 (2003).
65. Nakhoul, N. & Batuman, V. Role of proximal tubules in the pathogenesis of kidney disease. Contrib. Nephrol. 169, 37-50 (2011).
66. Chevalier, R. L. & Forbes, M. S. Generation and evolution of atubular glomeruli in the progression of renal disorders. J. Am. Soc. Nephrol. 19, 197-206 (2008).
67. Chevalier, R. L. The proximal tubule is the primary target of injury and progression of kidney disease: role of the glomerulotubular junction. Am. J. Physiol. Renal Physiol. 311, F145-61 (2016).
68. Grubman, A. et al. A single-cell atlas of entorhinal cortex from individuals with Alzheimer's disease reveals cell-type-specific gene expression regulation. Nat. Neurosci. 22, 2087-2097 (2019).
69. Dhillon, P. et al. The Nuclear Receptor ESRRA Protects from Kidney Disease by Coupling Metabolism and Differentiation. Cell Metab. 33, 379-394.e8 (2021).
70. Meex, R. C. R. & Watt, M. J. Hepatokines: linking nonalcoholic fatty liver disease and insulin resistance. Nat. Rev. Endocrinol. 13, 509-520 (2017).
71. McCall, M. A. et al. Targeted deletion in astrocyte intermediate filament (Gfap) alters neuronal physiology. Proc Natl Acad Sci USA 93, 6361-6366 (1996).
72. Lytton, J. Na+/Ca2+ exchangers: three mammalian gene families control Ca2+ transport. Biochem. J. 406, 365-382 (2007).
73. Friedman, L. G. et al. Cadherin-8 expression, synaptic localization, and molecular control of neuronal form in prefrontal corticostriatal circuits. J. Comp. Neurol. 523, 75-92 (2015).
74. Arlotta, P. et al. Neuronal subtype-specific genes that control corticospinal motor neuron development in vivo. Neuron 45, 207-221 (2005).
75. Shigemoto, R., Nakanishi, S. & Mizuno, N. Distribution of the mRNA for a metabotropic glutamate receptor (mGluR1) in the central nervous system: an in situ hybridization study in adult and developing rat. J. Comp. Neurol. 322, 121-135 (1992).
76. Zhou, Q., Choi, G. & Anderson, D. J. The bHLH transcription factor Olig2 promotes oligodendrocyte differentiation in collaboration with Nkx2.2. Neuron 31, 791-807 (2001).
77. Nielsen, J. A., Berndt, J. A., Hudson, L. D. & Armstrong, R. C. Myelin transcription factor 1 (Myt1) modulates the proliferation and differentiation of oligodendrocyte lineage cells. Mol. Cell. Neurosci. 25, 111-123 (2004).
78. Ichihara-Tanaka, K., Oohira, A., Rumsby, M. & Muramatsu, T. Neuroglycan C is a novel midkine receptor involved in process elongation of oligodendroglial precursor-like cells. J. Biol. Chem. 281, 30857-30864 (2006).
79. Levine, J. M., Reynolds, R. & Fawcett, J. W. The oligodendrocyte precursor cell in health and disease. Trends Neurosci. 24, 39-47 (2001).
80. Liddelow, S. A. et al. Neurotoxic reactive astrocytes are induced by activated microglia. Nature 541, 481-487 (2017).
81. Roadmap Epigenomics Consortium et al. Integrative analysis of 111 reference human epigenomes. Nature 518, 317-330 (2015).
82. Basu, M., Wang, K., Ruppin, E. & Hannenhalli, S. Predicting tissue-specific gene expression from whole blood transcriptome. Sci. Adv. 7, (2021).
83. Moufarrej, M. N., Wong, R. J., Shaw, G. M., Stevenson, D. K. & Quake, S. R. Investigating Pregnancy and Its Complications Using Circulating Cell-Free RNA in Women's Blood During Gestation. Front. Pediatr. 8, 605219 (2020).
84. Pan, W. Development of diagnostic methods using cell-free nucleic acids. (Stanford University, 2016).
85. Robinson, M. D. & Oshlack, A. A scaling normalization method for differential expression analysis of RNA-seq data. Genome Biol. 11, R25 (2010).
86. Wolf, F. A., Angerer, P. & Theis, F. J. SCANPY: large-scale single-cell gene expression data analysis. Genome Biol. 19, 15 (2018).
87. Shen-Orr, S. S., Tibshirani, R. & Butte, A. J. Gene expression deconvolution in linear space. Nat. Methods 9, 9-9 (2011).
88. Chang, C.-C. & Lin, C.-J. Training nu-support vector regression: theory and algorithms. Neural Comput. 14, 1959-1977 (2002).
89. Zhong, Y. & Liu, Z. Gene expression deconvolution in linear space. Nat. Methods 9, 8-9; author reply 9 (2012).
90. Pedregosa, F. et al. Scikit-learn: Machine Learning in Python. Journal of Machine Learning Research 12, 2825-2830 (2011).
91. Qiao, W. et al. PERT: a method for expression deconvolution of human blood samples from varied microenvironmental and developmental conditions. PLoS Comput. Biol. 8, e1002838 (2012).
92. van Rossum, D. & Hanisch, U.-K. Microglia. Metab. Brain Dis. 19, 393-411 (2004).

What is claimed is:

1. A method of evaluating the status of a cell type in a human, the method comprising,
providing a biological sample from the human,
detecting from the biological sample the presence, absence or quantity of cell-free RNA (cfRNA) from at least 3, 4, 5, 6, 7, 8, 9, 10, 11, 12, 13, 14, 15 or more or all indicative genes, wherein the cell type and indicative genes are selected from any one of Tables 1, 2, 3, 4, or 5; and
generating a score based on detection of the cfRNA from the cell type and indicative genes.

2. The method of claim 1, further comprising comparing the score to a control value.

3. The method of claim 2, wherein the control value is based on a set of control subjects.

4. The method of claim 1, comparing the score to a prior score from an earlier-obtained biological sample from the human.

5. The method of claim 1, further comprising detecting from the biological sample the presence, absence or quantity of cell-free RNA (cfRNA) from at least 3, 4, 5, 6, 7, 8, 9, 10, 11, 12, 13, 14, 15 or more or all indicative genes for a second cell type, wherein the indicative genes are selected from any one of Tables 1-5;
generating a second score based on detection of the cfRNA from the indicative genes for the second cell type; and
comparing or normalizing the score to the second score.

6. The method of any one of claims 2-5, further comprising starting, stopping or changing a treatment of the human based on the comparing.

7. A method of treating a disease or disorder in a human subject, the method comprising evaluating the status of a cell type in the human according to any one of claims 1-6, and administering at least one therapeutic agent or treatment to the human.

8. The method of claim 1, wherein the score is the sum of cfRNA copies detected for the indicative genes.

9. The method of any one of claims 1-8, wherein the biological sample is blood, urine, cerebrospinal fluid, interstitial fluid, amniotic fluid, cord blood, and/or semen.

10. A method of evaluating kidney function in a human, the method comprising,
providing a biological sample from the human,
detecting from the biological sample the presence, absence or quantity of cell-free RNA (cfRNA) from at least 3, 4, 5, 6, 7, 8, 9, 10, 11, 12, 13, 14, 15 or more or all indicative genes, wherein cell type and indicative genes are provided in any one or more of Tables 1-5 and Table 11;
generating a score based on detection of the cfRNA from the cell type and indicative genes;
comparing the score to a control value or a prior score from an earlier-obtained biological sample or from a score for a different cell type from the human, thereby evaluating kidney function in the human.

11. The method of claim 10, wherein the providing the biological sample from the human is non-invasive.

12. The method of claim 10, wherein the kidney function is indicative of prognosis or diagnosis for chronic kidney disease (CKD), acute kidney injury (AKI), and/or minimal change disease.

13. The method of claim 10, wherein the control value is based on a set of control subjects.

14. The method of any one of claims 10-13, further comprising starting, stopping or changing a treatment of the human based on the comparing.

15. The method of claim 10, wherein the score is the sum of cfRNA copies detected for the indicative genes.

16. The method of any one of claims 10-15, wherein the biological sample is blood or urine.

17. The method of any one of claims 10-16, wherein the comparing comprises comparing the score to a different cell type that is a intercalated cell, principal cell, loop of Henle cell, fibroblast, proximal tubule, podocyte, or hepatocyte.

18. The method of any one of claims 10-17, further comprising detecting serum creatinine, urine creatinine, urine protein, cystatin C, albuminuria, estimated glomerular filtration rate and/or glomerular filtration rate in the human.

19. A method of treating a kidney disease or disorder in a human patient, the method comprising evaluating kidney function in the human according to any one of claims 10-18, and administering at least one therapeutic agent or treatment to the human.

20. A method of evaluating brain function in a human, the method comprising,
providing a biological sample from the human,
detecting from the biological sample the presence, absence or quantity of cell-free RNA (cfRNA) from at least 3, 4, 5, 6, 7, 8, 9, 10, 11, 12, 13, 14, 15 or more or all indicative genes, wherein cell type and indicative genes are provided in any one or more of Tables 1-5, Table 6 and Table 8;
generating a score based on detection of the cfRNA from the cell type and indicative genes;
comparing the score to a control value or a prior score from an earlier-obtained biological sample from a score for a different cell type from the human, thereby evaluating brain function in the human.

21. The method of claim 20, wherein the providing the biological sample from the human is non-invasive.

22. The method of claim 20, wherein the brain function is indicative of prognosis or diagnosis for Alzheimer's disease.

23. The method of claim 20, wherein the control value is based on a set of control subjects.

24. The method of any one of claims 20-23, further comprising starting, stopping or changing treatment of the human based on the comparing.

25. The method of claim 20, wherein the score is the sum of cfRNA copies detected for the indicative genes.

26. The method of any one of claims 20-25, wherein the biological sample is blood or cerebrospinal fluid.

27. The method of any one of claims 20-26, wherein the comparing comprises comparing the score to a different cell type that is a glial (e.g., astrocyte, oligodendrocyte, oligodendrocyte precursor cell) or neuronal cell type (e.g., inhibitory or excitatory neurons).

28. The method of any one of claims 20-27, further comprising detecting or measuring congnition, Tau and/or amyloid beta in the human.

29. A method of treating a brain disease or disorder in a human patient, the method comprising evaluating brain function in the human according to any one of claims 20-27, and administering at least one therapeutic agent or treatment to the human.

30. A method of evaluating liver function in a human, the method comprising,
providing a biological sample from the human,
detecting from the biological sample the presence, absence or quantity of cell-free RNA (cfRNA) from at least 3, 4, 5, 6, 7, 8, 9, 10, or all indicative genes, wherein cell type and indicative genes are provided in any one or more of Tables 1-5 and Table 12;
generating a score based on detection of the cfRNA from the cell type and indicative genes;
comparing the score to a control value or a prior score from an earlier-obtained biological sample from a score for a different cell type from the human, thereby evaluating liver function in the human.

31. The method of claim 30, wherein the providing the biological sample from the human is non-invasive.

32. The method of claim 30, wherein the liver function is indicative of prognosis or diagnosis for non-alcoholic fatty liver disease, non-alcoholic steatohepatitis, and/or liver cancer.

33. The method of claim 30, wherein the control value is based on a set of control subjects.

34. The method of any one of claims 30-33, further comprising starting, stopping or changing a treatment of the human based on the comparing.

35. The method of claim 30, wherein the score is the sum of cfRNA copies detected for the indicative genes.

36. The method of any one of claims 30-35, wherein the biological sample is blood or urine.

37. The method of any one of claims 30-36, wherein the comparing comprises comparing the score to a different cell type that is a liver sinusoidal endothelial cell, a kidney cell, a neutrophil, an eosinophil, or a basophil.

38. The method of any one of claims 30-37, further comprising detecting Alanine transaminase (ALT), Aspartate transaminase (AST), Alkaline phosphatase (ALP), Albumin, total protein, Bilirubin, Gamma-glutamyltransferase (GGT), L-lactate dehydrogenase (LD), and/or Prothrombin time in the human.

39. A method of treating a liver disease or disorder in a human patient, the method comprising evaluating liver function in the human according to any one of claims 30-38, and administering at least one therapeutic agent or treatment to the human.

40. A non-transitory computer-readable storage device storing computer-executable instructions that, in response to execution, cause a processor to perform operations, the operations comprising:
receiving data indicating presence, absence or quantity of cell-free RNA (cfRNA) from at least 3, 4, 5, 6, 7, 8, 9, 10, 11, 12, 13, 14, 15, or more or all indicative genes for a cell type, wherein cell type and indicative genes are selected from any one of Table 1, 2, 3, 4, or 5;
generating a score based on detection of the cfRNA from the cell type and indicative genes;
comparing the score to a control value or a prior score from an earlier-obtained biological sample from the human,
upon determining that the score is above or below the control value or prior score, generating a classification of disease or prognosis of the human related to the cell type; and
displaying the classification.
